SEQUENCE LISTING (74 sequences)

SEQ ID NO: 1 - DNA, 1608 bp, Homo sapiens
atgctggggccctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctccgcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccggggcggtccgtggtccccgcgttgcttcctctgctggccgggaccctgctgctgctggagacggccactgctccctga

SEQ ID NO: 2 - protein, 535 aa, Homo sapiens
MLGPCMLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGRSVVPALLPLLAGTLLLLETATAP

SEQ ID NO: 3 - DNA, 1599 bp, Homo sapiens
atgcaggggccctgggtgctgctcctgctgggcctgaggctacagctctccctgggcatcatcccagttgaggaggagaacccggacttctggaaccgccaggcagccgaggccctgggtgccgccaagaagctgcagcctgcacagacagccgccaagaacctcatcatcttcctgggtgacgggatgggggtgtctacggtgacagctgccaggatcctaaaagggcagaagaaggacaaactggggcctgagaccttcctggccatggaccgcttcccgtacgtggctctgtccaagacatacagtgtagacaagcatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcaggaaagtcagtgggagtggtaaccaccacacgggtgcagcatgcctcgccagccggcgcctacgcccacacggtgaaccgcaactggtactcggatgccgacgtgcctgcctcggcccgccaggaggggtgccaggacatcgccacgcagctcatctccaacatggacattgatgtgatcctaggtggaggccgaaagtacatgtttcccatggggaccccagaccctgagtacccagatgactacagccaaggtgggaccaggctggacgggaagaatctggtgcaggaatggctggcgaagcaccagggtgcccggtacgtgtggaaccgcactgagctcctgcaggcttccctggacccgtctgtgacccatctcatgggtctctttgagcctggagacatgaaatacgagatccaccgagactccacactggacccctccctgatggagatgacagaggctgccctgctcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttacggagagcgagagcgggagccccgagtatcggcagcagtcagcagtgcccctggacggagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccgcgccggcaccaccgacgccgcgcacccggggccgtccgtggtccccgcgttgcttcctctgctggcagggaccttgctgctgctggggacggccactgctccctga

SEQ ID NO: 4 - protein, 532 aa, Homo sapiens
MQGPWVLLLLGLRLQLSLGIIPVEEENPDFWNRQAAEALGAAKKLQPAQTAAKNLIIFLGDGMGVSTVTAARILKGQKKDKLGPETFLAMDRFPYVALSKTYSVDKHVPDSGATATAYLCGVKGNFQTIGLSAAARFNQCNTTRGNEVISVMNRAKKAGKSVGVVTTTRVQHASPAGAYAHTVNRNWYSDADVPASARQEGCQDIATQLISNMDIDVILGGGRKYMFPMGTPDPEYPDDYSQGGTRLDGKNLVQEWLAKHQGARYVWNRTELLQASLDPSVTHLMGLFEPGDMKYEIHRDSTLDPSLMEMTEAALLLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDGETHAGEDVAVFARGPQAHLVHGVQEQTFIAHVMAFAACLEPYTACDLAPRAGTTDAAHPGPSVVPALLPLLAGTLLLLGTATAP

SEQ ID NO: 5 - protein, 123 aa, Artificial Sequence
  mu 12F3 vH
EVKLVESGGGLVQPGGSLSLSCAASGFTFTDYYMSWVRQPPGKALEWLALIRNKATGYTTEYSASVKGRFTISRDNSQSILYLQMNALRAEDSATYYCARASFYYDGKVLAYWGQGTLVTVSA

SEQ ID NO: 6 - protein, 101 aa, Artificial Sequence
  mu IGHV7-3.04 - closest murine germline V-gene
EVKLVESGGGLVQPGGSLSLSCAASGFTFTDYYMSWVRQPPGKALEWLALIRNKANGYTTEYSASVKGRFTISRDNSQSILYLQMNALRAEDSATYYCARD

SEQ ID NO: 7 - protein, 115 aa, Artificial Sequence
  hu IGHV3-49.01/hIGHJ4.01
EVQLVESGGGLVQPGRSLRLSCTASGFTFGDYAMSWFRQAPGKGLEWVGFIRSKAYGGTTEYTASVKGRFTISRDGSKSIAYLQMNSLKTEDTAVYYCTRYFDYWGQGTLVTVSS

SEQ ID NO: 8 - protein, 100 aa, Artificial Sequence
  hu IGHV3-72.01
EVQLVESGGGLVQPGGSLRLSCAASGFTFSDHYMDWVRQAPGKGLEWVGRTRNKANSYTTEYAASVKGRFTISRDDSKNSLYLQMNSLKTEDTAVYYCAR

SEQ ID NO: 9 - protein, 123 aa, Artificial Sequence
  h12F3 vHA
EVQLVESGGGLVQPGRSLRLSCTASGFTFGDYYMSWFRQAPGKGLEWVGLIRNKATGYTTEYSASVKGRFTISRDGSKSIAYLQMNSLKTEDTAVYYCTRASFYYDGKVLAYWGQGTLVTVSS

SEQ ID NO: 10 - protein, 123 aa, Artificial Sequence
  h12F3 vHB
EVQLVESGGGLVQPGRSLRLSCTASGFTFTDYYMSWFRQAPGKGLEWVGLIRNKATGYTTEYSASVKGRFTISRDNSKSIAYLQMNSLKTEDTAVYYCTRASFYYDGKVLAYWGQGTLVTVSS

SEQ ID NO: 11 - protein, 123 aa, Artificial Sequence
  h12F3 vHC
EVQLVESGGGLVQPGRSLRLSCTASGFTFTDYYMSWFRQAPGKGLEWLALIRNKATGYTTEYSASVKGRFTISRDNSKSIAYLQMNSLKTEDTAVYYCTRASFYYDGKVLAYWGQGTLVTVSS

SEQ ID NO: 12 - protein, 123 aa, Artificial Sequence
  h12F3 vHD
EVQLVESGGGLVQPGRSLRLSCTASGFTFTDYYMSWFRQAPGKGLEWVGLIRNKATGYTTEYSASVKGRFTISRDNSKSILYLQMNSLKTEDTAVYYCARASFYYDGKVLAYWGQGTLVTVSS

SEQ ID NO: 13 - protein, 123 aa, Artificial Sequence
  h12F3 vHE
EVQLVESGGGLVQPGRSLRLSCTASGFTFTDYYMSWFRQAPGKGLEWLALIRNKATGYTTEYSASVKGRFTISRDNSKSILYLQMNSLKTEDTAVYYCARASFYYDGKVLAYWGQGTLVTVSS

SEQ ID NO: 14 - protein, 123 aa, Artificial Sequence
  h12F3 vHF
EVQLVESGGGLVQPGRSLRLSCTASGFTFTDYYMSWVRQAPGKGLEWLALIRNKATGYTTEYSASVKGRFTISRDNSKSILYLQMNSLKTEDTAVYYCARASFYYDGKVLAYWGQGTLVTVSS

SEQ ID NO: 15 - protein, 123 aa, Artificial Sequence
  h12F3 vHG
EVQLVESGGGLVQPGRSLRLSCTASGFTFTDYYMSWVRQAPGKGLEWLALIRNKATGYTTEYTASVKGRFTISRDNSKSILYLQMNSLKTEDTAVYYCARASFYYDGKVLAYWGQGTLVTVSS

SEQ ID NO: 16 - protein, 123 aa, Artificial Sequence
  h12F3 vHH
EVQLVESGGGLVQPGRSLRLSCTASGFTFTDYYMSWVRQAPGKGLEWLALIRNKATGYTTEYTASVKGRFTISRDNSKNSLYLQMNSLKTEDTAVYYCARASFYYDGKVLAYWGQGTLVTVSS

SEQ ID NO: 17 - protein, 106 aa, Artificial Sequence
  mu 12F3 vL
DTQMTQSPSSLSASLGGKVTITCKASQDINKYIAWYQYKTGKGPRLLIHYTSTLQPGIPSRFSGSGSGRDYSFSISNLEPEDIATYYCLQYDNLYTFGGGTKLEIK

SEQ ID NO: 18 - protein, 95 aa, Artificial Sequence
  mu IGKV19-93.01 - closest murine germline V-gene
DIQMTQSPSSLSASLGGKVTITCKASQDINKYIAWYQHKPGKGPRLLIHYTSTLQPGIPSRFSGSGSGRDYSFSISNLEPEDIATYYCLQYDNLL

SEQ ID NO: 19 - protein, 107 aa, Artificial Sequence
  hu IGKV1-33.01/hIGKJ2.01
DIQMTQSPSSLSASVGDRVTITCQASQDISNYLNWYQQKPGKAPKLLIYDASNLETGVPSRFSGSGSGTDFTFTISSLQPEDIATYYCQQYDNLPYTFGQGTKLEIK

SEQ ID NO: 20 - protein, 95 aa, Artificial Sequence
  hu IGKV1D-43.01
AIRMTQSPFSLSASVGDRVTITCWASQGISSYLAWYQQKPAKAPKLFIYYASSLQSGVPSRFSGSGSGTDYTLTISSLQPEDFATYYCQQYYSTP

SEQ ID NO: 21 - protein, 95 aa, Artificial Sequence
  hu IGKV1-16.01
DIQMTQSPSSLSASVGDRVTITCRASQGISNYLAWFQQKPGKAPKSLIYAASSLQSGVPSRFSGSGSGTDFTLTISSLQPEDFATYYCQQYNSYP

SEQ ID NO: 22 - protein, 106 aa, Artificial Sequence
  h12F3 vL1
DIQMTQSPSSLSASVGDRVTITCQASQDINKYLNWYQQKPGKAPKLLIYYTSNLETGVPSRFSGSGSGTDFTFTISSLQPEDIATYYCLQYDNLYTFGQGTKLEIK

SEQ ID NO: 23 - protein, 106 aa, Artificial Sequence
  h12F3 vL2
DIQMTQSPSSLSASVGDRVTITCQASQDINKYLNWYQQKPGKAPKLLIYYTSSLQSGVPSRFSGSGSGTDFTFTISSLQPEDIATYYCLQYDNLYTFGQGTKLEIK

SEQ ID NO: 24 - protein, 106 aa, Artificial Sequence
  h12F3 vL3
DIQMTQSPSSLSASVGDRVTITCQASQDINKYLAWYQQKPGKAPKLLIYYTSSLQPGVPSRFSGSGSGTDFTFTISSLQPEDIATYYCLQYDNLYTFGQGTKLEIK

SEQ ID NO: 25 - protein, 106 aa, Artificial Sequence
  h12F3 vLA
DIQMTQSPSSLSASVGDRVTITCKASQDINKYIAWYQQKPGKAPKLLIYYTSTLQPGVPSRFSGSGSGTDFTFTISSLQPEDIATYYCLQYDNLYTFGQGTKLEIK

SEQ ID NO: 26 - protein, 106 aa, Artificial Sequence
  h12F3 vLB
DTQMTQSPSSLSASVGDRVTITCKASQDINKYIAWYQQKPGKAPKLLIHYTSTLQPGVPSRFSGSGSGRDYTFTISSLQPEDIATYYCLQYDNLYTFGQGTKLEIK

SEQ ID NO: 27 - protein, 106 aa, Artificial Sequence
  h12F3 vLC
DTQMTQSPSSLSASVGDRVTITCKASQDINKYIAWYQQKPGKAPKLLIYYTSTLQPGVPSRFSGSGSGTDYTFTISSLQPEDIATYYCLQYDNLYTFGQGTKLEIK

SEQ ID NO: 28 - protein, 106 aa, Artificial Sequence
  h12F3 vLD
DTQMTQSPSSLSASVGDRVTITCQASQDINKYIAWYQQKPGKAPKLLIYYTSSLQPGVPSRFSGSGSGTDYTFTISSLQPEDIATYYCLQYDNLYTFGQGTKLEIK

SEQ ID NO: 29 - protein, 106 aa, Artificial Sequence
  h12F3 vLE
DTQMTQSPSSLSASVGDRVTITCQASQDINKYIAWYQQKPGKAPKLLIHYTSSLQSGVPSRFSGSGSGRDYTFTISSLQPEDIATYYCLQYDNLYTFGQGTKLEIK

SEQ ID NO: 30 - protein, 106 aa, Artificial Sequence
  h12F3 vLF
DTQMTQSPSSLSASVGDRVTITCQASQDINKYLAWYQYKPGKAPKLLIHYTSSLQSGVPSRFSGSGSGRDYTFTISSLQPEDIATYYCLQYDNLYTFGQGTKLEIK

SEQ ID NO: 31 - protein, 106 aa, Artificial Sequence
  h12F3 vLG
DTQMTQSPSSLSASVGDRVTITCQASQDINKYLAWYQQKTGKAPKLLIHYTSSLQSGVPSRFSGSGSGRDYTFTISSLQPEDIATYYCLQYDNLYTFGQGTKLEIK

SEQ ID NO: 32 - protein, 106 aa, Artificial Sequence
  h12F3 vLH
DTQMTQSPSSLSASVGDRVTITCKASQDINKYIAWYQYKTGKAPKLLIHYTSTLQPGVPSRFSGSGSGRDYTFTISSLQPEDIATYYCLQYDNLYTFGQGTKLEIK

SEQ ID NO: 33 - protein, 106 aa, Artificial Sequence
  h12F3 vLI
DTQMTQSPSSLSASVGDRVTITCKASQDINKYIAWFQYKTGKAPKLFIHYTSTLQPGVPSRFSGSGSGRDYTLTISSLQPEDIATYYCLQYDNLYTFGQGTKLEIK

SEQ ID NO: 34 - protein, 453 aa, Artificial Sequence
  h12F3 HA heavy chain
EVQLVESGGGLVQPGRSLRLSCTASGFTFGDYYMSWFRQAPGKGLEWVGLIRNKATGYTTEYSASVKGRFTISRDGSKSIAYLQMNSLKTEDTAVYYCTRASFYYDGKVLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 453 aa, Artificial Sequence
  h12F3 HB heavy chain
EVQLVESGGGLVQPGRSLRLSCTASGFTFTDYYMSWFRQAPGKGLEWVGLIRNKATGYTTEYSASVKGRFTISRDNSKSIAYLQMNSLKTEDTAVYYCTRASFYYDGKVLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 453 aa, Artificial Sequence
  h12F3 HC heavy chain
EVQLVESGGGLVQPGRSLRLSCTASGFTFTDYYMSWFRQAPGKGLEWLALIRNKATGYTTEYSASVKGRFTISRDNSKSIAYLQMNSLKTEDTAVYYCTRASFYYDGKVLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 453 aa, Artificial Sequence
  h12F3 HD heavy chain
EVQLVESGGGLVQPGRSLRLSCTASGFTFTDYYMSWFRQAPGKGLEWVGLIRNKATGYTTEYSASVKGRFTISRDNSKSILYLQMNSLKTEDTAVYYCARASFYYDGKVLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 453 aa, Artificial Sequence
  h12F3 HE heavy chain
EVQLVESGGGLVQPGRSLRLSCTASGFTFTDYYMSWFRQAPGKGLEWLALIRNKATGYTTEYSASVKGRFTISRDNSKSILYLQMNSLKTEDTAVYYCARASFYYDGKVLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 453 aa, Artificial Sequence
  h12F3 HF heavy chain
EVQLVESGGGLVQPGRSLRLSCTASGFTFTDYYMSWVRQAPGKGLEWLALIRNKATGYTTEYSASVKGRFTISRDNSKSILYLQMNSLKTEDTAVYYCARASFYYDGKVLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 453 aa, Artificial Sequence
  h12F3 HG heavy chain
EVQLVESGGGLVQPGRSLRLSCTASGFTFTDYYMSWVRQAPGKGLEWLALIRNKATGYTTEYTASVKGRFTISRDNSKSILYLQMNSLKTEDTAVYYCARASFYYDGKVLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 453 aa, Artificial Sequence
  h12F3 HH heavy chain
EVQLVESGGGLVQPGRSLRLSCTASGFTFTDYYMSWVRQAPGKGLEWLALIRNKATGYTTEYTASVKGRFTISRDNSKNSLYLQMNSLKTEDTAVYYCARASFYYDGKVLAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 42 - protein, 213 aa, Artificial Sequence
  h12F3 L1 light chain
DIQMTQSPSSLSASVGDRVTITCQASQDINKYLNWYQQKPGKAPKLLIYYTSNLETGVPSRFSGSGSGTDFTFTISSLQPEDIATYYCLQYDNLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 213 aa, Artificial Sequence
  h12F3 L2 light chain
DIQMTQSPSSLSASVGDRVTITCQASQDINKYLNWYQQKPGKAPKLLIYYTSSLQSGVPSRFSGSGSGTDFTFTISSLQPEDIATYYCLQYDNLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 44 - protein, 213 aa, Artificial Sequence
  h12F3 L3 light chain
DIQMTQSPSSLSASVGDRVTITCQASQDINKYLAWYQQKPGKAPKLLIYYTSSLQPGVPSRFSGSGSGTDFTFTISSLQPEDIATYYCLQYDNLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 213 aa, Artificial Sequence
  h12F3 LA light chain
DIQMTQSPSSLSASVGDRVTITCKASQDINKYIAWYQQKPGKAPKLLIYYTSTLQPGVPSRFSGSGSGTDFTFTISSLQPEDIATYYCLQYDNLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 213 aa, Artificial Sequence
  h12F3 LB light chain
DTQMTQSPSSLSASVGDRVTITCKASQDINKYIAWYQQKPGKAPKLLIHYTSTLQPGVPSRFSGSGSGRDYTFTISSLQPEDIATYYCLQYDNLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 213 aa, Artificial Sequence
  h12F3 LC light chain
DTQMTQSPSSLSASVGDRVTITCKASQDINKYIAWYQQKPGKAPKLLIYYTSTLQPGVPSRFSGSGSGTDYTFTISSLQPEDIATYYCLQYDNLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 213 aa, Artificial Sequence
  h12F3 LD light chain
DTQMTQSPSSLSASVGDRVTITCQASQDINKYIAWYQQKPGKAPKLLIYYTSSLQPGVPSRFSGSGSGTDYTFTISSLQPEDIATYYCLQYDNLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 213 aa, Artificial Sequence
  h12F3 LE light chain
DTQMTQSPSSLSASVGDRVTITCQASQDINKYIAWYQQKPGKAPKLLIHYTSSLQSGVPSRFSGSGSGRDYTFTISSLQPEDIATYYCLQYDNLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 213 aa, Artificial Sequence
  h12F3 LF light chain
DTQMTQSPSSLSASVGDRVTITCQASQDINKYLAWYQYKPGKAPKLLIHYTSSLQSGVPSRFSGSGSGRDYTFTISSLQPEDIATYYCLQYDNLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 213 aa, Artificial Sequence
  h12F3 LG light chain
DTQMTQSPSSLSASVGDRVTITCQASQDINKYLAWYQQKTGKAPKLLIHYTSSLQSGVPSRFSGSGSGRDYTFTISSLQPEDIATYYCLQYDNLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 213 aa, Artificial Sequence
  h12F3 LH light chain
DTQMTQSPSSLSASVGDRVTITCKASQDINKYIAWYQYKTGKAPKLLIHYTSTLQPGVPSRFSGSGSGRDYTFTISSLQPEDIATYYCLQYDNLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 53 - protein, 213 aa, Artificial Sequence
  h12F3 LI light chain
DTQMTQSPSSLSASVGDRVTITCKASQDINKYIAWFQYKTGKAPKLFIHYTSTLQPGVPSRFSGSGSGRDYTLTISSLQPEDIATYYCLQYDNLYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 330 aa, Artificial Sequence [sequence omitted]

SEQ ID NO: 55 - protein, 107 aa, Artificial Sequence [sequence omitted]

SEQ ID NO: 56 - protein, 5 aa, Artificial Sequence
  HF, HG CDR 1, Kabat
DYYMS

SEQ ID NO: 57 - protein, 19 aa, Artificial Sequence
  HG CDR 2, Kabat
LIRNKATGYTTEYTASVKG

SEQ ID NO: 58 - protein, 12 aa, Artificial Sequence
  HF, HG CDR 3, Kabat
ASFYYDGKVLAY

SEQ ID NO: 59 - protein, 19 aa, Artificial Sequence
  HF CDR 2, Kabat
LIRNKATGYTTEYSASVKG

SEQ ID NO: 60 - protein, 8 aa, Artificial Sequence
  HF, HG CDR 1, IMGT
GFTFTDYY

SEQ ID NO: 61 - protein, 10 aa, Artificial Sequence
  HF, HG CDR 2, IMGT
IRNKATGYTT

SEQ ID NO: 62 - protein, 14 aa, Artificial Sequence
  HF, HG CDR 3, IMGT
ARASFYYDGKVLAY

SEQ ID NO: 63 - protein, 11 aa, Artificial Sequence
  LF CDR 1, Kabat
QASQDINKYLA

SEQ ID NO: 64 - protein, 7 aa, Artificial Sequence
  LF CDR 2, Kabat
YTSSLQS

SEQ ID NO: 65 - protein, 8 aa, Artificial Sequence
  LD, LF CDR 3, Kabat
LQYDNLYT

SEQ ID NO: 66 - protein, 11 aa, Artificial Sequence
  LD CDR 1, Kabat
QASQDINKYIA

SEQ ID NO: 67 - protein, 7 aa, Artificial Sequence
  LD CDR 2, Kabat
YTSSLQP

SEQ ID NO: 68 - protein, 6 aa, Artificial Sequence
  LD, LF CDR 1, IMGT
QDINKY

SEQ ID NO: 69 - protein, 3 aa, Artificial Sequence
  LD, LF CDR 2, IMGT
YTS

SEQ ID NO: 70 - protein, 8 aa, Artificial Sequence
  LD, LF CDR 3, IMGT
LQYDNLYT

SEQ ID NO: 71 - DNA, 369 bp, Artificial Sequence
  h12F3 vHG Nucleic Acid Sequence
gaggtgcagctggtggagtccggaggaggactggtgcagcccggtcgttctttaaggctgagctgcacagccagcggcttcaccttcaccgactactacatgtcttgggtgaggcaagctcccggtaagggactggagtggctggctttaattcgtaacaaggccaccggctacaccaccgagtacaccgcctccgtgaagggtcgtttcaccatctctcgtgacaacagcaagtccattttatatttacagatgaactctttaaagaccgaggacaccgccgtgtactactgcgctcgtgcctccttttactacgacggcaaggtgctggcctactggggccaaggtactttagtgaccgtgtcctcc

SEQ ID NO: 72 - DNA, 319 bp, Artificial Sequence
  h12F3 vLF Nucleic Acid Sequence
gacacccagatgacccagtccccttcctctttatccgcttccgtgggagatcgtgtgaccatcacttgtcaagcttcccaagatatcaacaagtacctggcttggtaccagtacaagcccggcaaggcccccaagctgctgatccactacacctcctctttacagtccggagtgccttctcgtttctccggctccggaagcggtcgtgactacaccttcaccatctcctctttacagcccgaggacatcgctacctactactgtttacagtacgacaatttatacaccttcggccaaggtaccaagctggagatcaagc

SEQ ID NO: 73 - protein, 43 aa, Artificial Sequence
  h12F3 epitope--ALPP
LDPSVTHLMGLFEPGDMKYEIHRDSTLDPSLMEMTEAALRLLS

SEQ ID NO: 74 - protein, 43 aa, Artificial Sequence
  h12F3 epitope-ALPPL2
LDPSVTHLMGLFEPGDMKYEIHRDSTLDPSLMEMTEAALLLLS

(19) United States
(12) Patent Application Publication
(10) Pub. No.: US 2025/0276076 A1
(43) Pub. Date: Sep. 4, 2025

(54) ANTI-ALPP/ALPPL2 ANTIBODIES AND ANTIBODY-DRUG CONJUGATES

(71) Applicant: SEAGEN INC., Bothell, WA (US)

(72) Inventors: Hector De Jesus Rincon Arano, Shoreline, WA (US); Sarah Ann Riordan Anderson, Seattle, WA (US); Erin Margaret Scherer, Decatur, GA (US); Lori Westendorf, Snohomish, WA (US); Noah Bindman, Seattle, WA (US); Nicole Okeley, Redmond, WA (US); Peter Senter, Seattle, WA (US); Divya Awasthi, Seattle, WA (US)

(73) Assignee: SEAGEN INC., Bothell, WA (US)

(21) Appl. No.: 18/282,322

(22) PCT Filed: Mar. 17, 2022
(86) PCT No.: PCT/US2022/020697

§ 371 Date: Sep. 15, 2023

Related U.S. Application Data
(60) Provisional application No. 63/162,635, filed on Mar. 18, 2021.
(60) Provisional application No. 63/301,574, filed on Jan. 21, 2022.

(51) Int. Cl. A61K 47/68; A61K 39/00; A61P 35/00; C07K 16/40; C12N 15/85

(52) U.S. Cl. CPC A61K 47/68031; A61K 47/6871; A61K 47/6889; A61P 35/00; C07K 16/40; C12N 15/85; A61K 2039/505; C07K 2317/14; C07K 2317/24; C07K 2317/565

(57) ABSTRACT

Antigen binding proteins such as antibodies and fragments thereof that bind ALPP and/or ALPPL2 are provided. Nucleic acids encoding such antigen binding proteins and vectors and cells useful in preparing such antigen binding proteins are also provided. The antigen binding proteins are useful in a variety of methods, including the treatment of ovarian cancer.

### CROSS REFERENCE TO RELATED APPLICATION

This application claims the benefit of U.S. Provisional Application No. 63/162,635 filed Mar. 18, 2021 and U.S. Provisional Application No. 63/301,574 filed Jan. 21, 2022, each of which are incorporated by reference in their entirety for all purposes.

### REFERENCE TO A SEQUENCE LISTING

This application includes an electronic sequence listing in a file named 5620-00112PC_ST25 created on Mar. 4, 2022 and containing 106 Kb, which is hereby incorporated by reference.

### TECHNICAL FIELD

The present invention relates to novel anti-ALPP/ALPPL2 antibodies and antibody-drug conjugates and methods of using such anti-ALPP/ALPPL2 antibodies and antibody-drug conjugates to treat cancer.

### BACKGROUND

ALPP, which is also known as placental alkaline phosphatase, and ALPPL2, which is also known as placental like 2 alkaline phosphatase, are paralogous genes largely expressed in placenta. ALPP and ALPPL2 are membrane-bound proteins involved in ATP recycling from the extracellular space. ALPP is upregulated in multiple cancers, including ovarian cancer, lung cancer, endometrial cancer, bladder cancer, and gastric cancer. ALPPL2 is also upregulated in multiple cancers, including ovarian cancer, lung cancer, endometrial cancer, bladder cancer, gastric cancer, and testicular cancer. Ovarian cancer is the fifth most common gynecologic malignancy, and there is a need for improved treatments for this disease.

All references cited herein, including patent applications, patent publications, and scientific literature, are herein incorporated by reference in their entirety, as if each individual reference were specifically and individually indicated to be incorporated by reference.

### SUMMARY

Provided herein are anti-ALPP antibodies and ALPP-directed antibody-drug conjugates (ADCs), and anti-ALPPL2 antibodies and ALPPL2-directed ADCs. Also provided herein are antibodies that can bind both ALPP and ALPPL2 (anti-ALPP/ALPPL2 antibodies) and ADCs directed to both ALPP and ALPPL2 (ALPP/ALPPL2-directed ADCs). Also provided herein are methods of using anti-ALPP/ALPPL2-directed antibodies and ADCs to treat ALPP and/or ALPPL2-expressing disorders, including cancers. In some embodiments, the anti-ALPP/ALPPL2 antibodies comprise heavy chain CDR sequences of SEQ ID NOs: 56, 57, and 58, and light chain CDR sequences of SEQ ID NOs: 63, 64, and 65, as determined by Kabat numbering. In some embodiments, the anti-ALPP/ALPPL2 antibodies comprise heavy chain CDR sequences of SEQ ID NOs: 60, 61, and 62, and light chain CDR sequences of SEQ ID NOs: 66, 67, and 68, as determined by IMGT numbering.

### BRIEF DESCRIPTION OF THE DRAWINGS

FIG. 1 shows a cytotoxicity evaluation of ALPP/ALPPL2-specific antibodies as ADCs using payloads with and without bystander activity on COV644 and NCI-H1651 tumor cells.

FIGS. 2A to 2C show the remaining viability of the cell lines CAOV3, COV644 and NCI-H1651 upon cytotoxicity evaluation of top ALPP/ALPPL2-specific antibodies as ADCs using payloads without bystander activity.

FIG. 3 shows the binding affinities of ALPP/ALPPL2 specific antibodies.

FIG. 4 shows the full binding profile for antibodies 1F7 and 12F3 to both ALPP and ALPPL2 expressing HEK293 cells by flow cytometry.

FIG. 5 shows the sequence alignment of h12F3 variable heavy chain variants with human heavy acceptor sequence, IGHV3-49/HJ4.

FIG. 6 shows the variable domain alignment of h12F3 variable heavy chain variants.

FIG. 7 shows the sequence alignment of h12F3 variable light chain variants with human kappa acceptor sequence, IGKV1-33/KJ2.

FIG. 8 shows the variable domain alignment of h12F3 light chain variants.

FIG. 9 shows the percentage of viable CAOV3 cells. The left side of the figure shows the percentage of viability of a dose-response curve of ADCs containing humanized F light chain variants with different heavy chains. The right side of the figure shows the viability for ADCs containing the humanized D heavy chain with different light chain variants.

FIG. 10 shows the in vitro potency correlation between humanized variants having the mp-dLAE-MMAE(4) drug linker.

FIG. 11 shows the cytotoxicity profile of h12F3 HGLF ADCs on 3D spheroids in vitro. shows kinetic binding curves and values for both h12F3 HGLF and HFLD variants to ALPP and ALPPL2 Fc fusions.

FIG. 12 shows the internalization kinetics of h12F3 HGLF at various antibody concentrations.

FIG. 13 shows kinetic binding curves and values for both h12F3 HGLF and HFLD variants to ALPP and ALPPL2 Fc fusions.

FIG. 14 shows kinetic binding curves and values at pH 7.4 and pH 6 for h12F3 HGLF and HFLD to ALPP and ALPPL2 fc fusions.

FIG. 15 shows in vivo antitumor activity of h12F3 HGLF-dLAE-MMAE and HFLD-dLAE-MMAE in CAOV3 mouse models.

FIG. 16 shows in vivo antitumor activity of h12F3 HGLF-dLAE-MMAE and HFLD-dLAE-MMAE in NCI-N87 mouse models.

FIG. 17 shows in vivo antitumor activity of h12F3 HFLD-dLAE-MMAE and HFLD-vc-MMAE in NCI-N87 mouse models.

FIG. 18 shows in vivo antitumor activity of h12F3 HFLD-dLAE-MMAE and HFLD-vc-MMAE in H1651 mouse models.

FIG. 19 shows the % tumor volume change across seven xenograft models upon treatment with h12F3 ADCs conjugated to vc-MMAE and dLAE-MMAE.

FIG. 20 shows in vivo antitumor activity in the NCI-N87 gastric model by h12F3 HGLF-mc-vc-MMAE or h12F3 HGLF-mp-dLAE-MMAE conjugates in comparison to their respective isotype ADC controls.

FIG. 21 shows in vivo antitumor activity in the NCI-N87 gastric model by h12F3 HGLF-mc-vc-MMAE or h12F3 HGLF-mp-dLAE-MMAE conjugates in comparison to their respective isotype ADC controls.

FIG. 22 shows a summary of tumor growth inhibition across four xenograft tumor models including pancreatic (HPAC), gastric (NCI-N87), ovarian (CAOV3) and lung (SNU-2535) by h12F3-HGLF me-vc-MMAE and mp-dLAE-MMAE. Average non-target ADC shown as a dashed line.

FIG. 23 shows antitumor activity on ovarian patient-derived xenografts treated with h12F3-HDLF-mc-vc-MMAE. A) shows a summary of tumor growth inhibition across twelve xenografts, while B) and C) show examples of two conjugate-treated models compared to untreated cohorts.

FIG. 24 shows binding affinities of h12F3 HGLF and HFLD for human ALPP and monkey ortholog ALPP.

FIG. 25 shows epitope mapping of h12F3 HGLF to chimeric rat/human ALPP expressing HEK293 cells.

FIG. 26 shows sensorgrams showing the binding of h12F3, h12F3-HGLF-mc-vc-MMAE, h12F3-mp-dLAE-MMAE or positive control mAb (varied by row) to human Fc receptors (varied by column). Equilibrium dissociation constants are listed in the top right corner of each sensorgram.

FIG. 27 shows cell lysis of LoVo cells determined by chromium release assay after incubating Na2[51Cr]O4 (Cr-51)-labeled cells with NK cells in the presence of h12F3 HGLF, h12F3 HGLF-mc-vc-MMAE, or h12F3 HGLF-mp-dLAE-MMAE conjugates.

FIG. 28 shows phagocytic activity of macrophages incubated with LoVo cells in the presence of h12F3 HGLF, h12F3 HGLF-mc-vc-MMAE, or h12F3 HGLF-mp-dLAE-MMAE conjugates in comparison to positive (function-blocking anti-CD47 antibody) or isotyope control.

FIG. 29 shows the FcgRIII mediated activation of luminescent reporter by h12F3 HGLF antibody and ADCs.

FIG. 30 shows activation of two signaling pathways involved in immunogenic cell death by h12F3 HGLF-mc-vc-MMAE, or h12F3 HGLF-mp-dLAE-MMAE conjugates at two different concentrations.

FIG. 31 shows the release of ATP in media by LoVo cells treated with 1 or 10 mg/ml of h12F3 HGLF-mc-vc-MMAE or h12F3 HGLF-mp-dLAE-MMAE conjugates in comparison to free MMAE cytotoxin for 24 or 48 h.

### DETAILED DESCRIPTION

The section headings used herein are for organizational purposes only and are not to be construed as limiting the subject matter described.

The techniques and procedures described or referenced herein are generally well understood and commonly employed using conventional methodology by those skilled in the art, such as, for example, the widely utilized methodologies described in Sambrook et al., Molecular Cloning: A Laboratory Manual 4th edition (2012) Cold Spring Harbor Laboratory Press, Cold Spring Harbor N.Y.; Current Protocols In Molecular Biology (F. M. Ausubel, et al. eds., (2003)); the series METHODS IN ENZYMOLOGY (Academic Press, Inc.); PCR 2: A Practical Approach (M. J. MacPherson, B. D. Hames and G. R. Taylor eds. (1995)); Greenfield, ed. (2013) Antibodies, A Laboratory Manual, 2^{nd} edition, Cold Spring Harbor Laboratory Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); Cancer: Principles and Practice of Oncology (V. T. DeVita et al., eds., J.B. Lippincott Company, 1993); and updated versions thereof. Each of the foregoing references in this paragraph is incorporated herein by reference in its entirety.

### I. Definitions

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 5^{th} ed., 2013, Academic Press; and the Oxford Dictionary Of Biochemistry And Molecular Biology, 2^{nd} ed., 2006, Oxford University Press, provide one of skill with a general dictionary of many of the terms used in this disclosure.

Unless otherwise required by context or expressly indicated, singular terms shall include pluralities and plural terms shall include the singular.

It is understood that aspect and embodiments of the invention described herein include “comprising,” “consisting,” and/or “consisting essentially of” aspects and embodiments.

As used herein, the singular form “a”, “an”, and “the” should be understood to refer to “one or more” of any recited or enumerated component unless indicated otherwise.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about” refers to a value or composition that is within an acceptable error range for the particular value or composition as determined by one of ordinary skill in the art, which will depend in part on how the value or composition is measured or determined, i.e., the limitations of the measurement system. As is understood by one skilled in the art, reference to “about” a value or parameter herein includes (and describes) embodiments that are directed to that value or parameter per se. For example, description referring to “about X” includes description of “X”.

As described herein, any concentration range, percentage range, ratio range, or integer range is to be understood to include the value of any integer within the recited range and, when appropriate, fractions thereof (such as one tenth and one hundredth of an integer), unless otherwise indicated.

When a trade name is used herein, reference to the trade name also refers to the product formulation, the generic drug, and the active pharmaceutical ingredient(s) of the trade name product, unless otherwise indicated by context.

The terms “ALPP,” “alkaline phosphatase,” “alkaline phosphatase, placental,” “ALPase,” or “PLAP” are used interchangeably herein, and, unless otherwise specified, include any naturally occurring variants (e.g., splice variants, allelic variants), isoforms, and vertebrate species homologs of human ALPP. The term encompasses “full length,” unprocessed ALPP as well as any form of ALPP that results from processing within a cell. The amino acid sequence of an exemplary human ALPP is provided in Uniprot ID: P05187 or RefSeq ID: NM_001632. The amino acid sequence of one specific example of a mature human ALPP protein is set forth in SEQ ID NO: 2.

The terms “ALPPL2,” “alkaline phosphatase, placental-like 2,” or “alkaline phosphatase, germ cell” are used interchangeably herein, and, unless otherwise specified, include any naturally occurring variants (e.g., splice variants, allelic variants), isoforms, and vertebrate species homologs of human ALPPL2. The term encompasses “full length,” unprocessed ALPPL2 as well as any form of ALPPL2 that results from processing within a cell. The amino acid sequence of an exemplary human ALPPL2 is provided in Uniprot ID: P10696 or RefSeq ID: NM_031313. The amino acid sequence of one specific example of a mature human ALPPL2 protein is set forth in SEQ ID NO: 4.

An “antigen binding protein” (“ABP”) as used herein means any protein that binds a specified target antigen other than the naturally occurring cognate ligand(s) or fragments of such ligand(s) that bind the specified antigen. In the instant application, the specified target antigen is ALPP and/or ALPPL2 or a fragment of ALPP and/or ALPPL2. An “antigen binding protein” includes proteins that include at least one antigen binding region or domain (e.g., at least one hypervariable region (HVR) or complementarity determining region (CDR) as defined herein). In some embodiments, an antigen binding protein comprises a scaffold, such as a polypeptide or polypeptides, into which one or more (e.g., 1, 2, 3, 4, 5 or 6) HVR(s) or CDR(s), as described herein, are embedded and/or joined. In some antigen binding proteins, the HVRs or CDRs are embedded into a “framework” region, which orients the HVR(s) or CDR(s) such that the proper antigen binding properties of the CDR(s) are achieved. For some antigen binding proteins, the scaffold is the immunoglobulin heavy and/or light chain(s) from an antibody or a fragment thereof. Additional examples of scaffolds include, but are not limited to, human fibronectin (e.g., the 10^{th} extracellular domain of human fibronectin III), neocarzinostatin CBM4-2, anticalines derived from lipocalins, designed ankyrin repeat domains (DARPins), protein-A domain (protein Z), Kunitz domains, Im9, TPR proteins, zinc finger domains, pVIII, GC4, transferrin, B-domain of SPA, Sac7d, A-domain, SH3 domain of Fyn kinase, and C-type lectin-like domains (see, e.g., Gebauer and Skerra (2009) Curr. Opin. Chem. Biol., 13:245-255; Binz et al. (2005) Nat. Biotech. 23:1257-1268; and Yu et al. (2017) Annu Rev Anal Chem 10:293-320, each of which is incorporated herein by reference in its entirety). Accordingly, antigen binding proteins include, but are not limited to, monoclonal antibodies, bispecific antibodies, minibodies, domain antibodies such as Nanobodies®, synthetic antibodies (sometimes referred to herein as “antibody mimetics”), chimeric antibodies, humanized antibodies, human antibodies, antibody fusions, and portions or fragments of each, respectively. In some instances, the antigen binding protein is a functional fragment of a complete antibody (e.g., a Fab, a Fab′, a F(ab′)₂, a scFv, a domain antibody or a minibody). Peptibodies are another example of antigen binding proteins. In some embodiments, the term “antigen binding protein” includes derivatives, for example an antigen binding protein that has been chemically-modified, for example an antigen binding protein that is joined to another agent such as a label or a cytotoxic or cytostatic agent (e.g., an antigen binding protein conjugate such as an antibody drug conjugate).

An “antigen-binding fragment” (or simply “fragment”) or “antigen-binding domain” of an antigen binding protein (e.g., an antibody) as used herein refers to one or more fragments of an antigen binding protein (e.g., an antibody), regardless of how obtained or synthesized, that retain the ability to specifically bind to the antigen bound by the whole antigen binding protein. Examples of antibody fragments include, but are not limited to, Fv; Fab; Fab′; Fab′-SH; F(ab′)₂; diabodies; linear antibodies; single-chain antibody molecules (e.g. scFv); and multispecific antibodies formed from antibody fragments. A “Fv” fragment includes a non-covalently-linked dimer of one heavy chain variable domain and one light chain variable domain. A “Fab” fragment includes, the constant domain of the light chain and the first constant domain (C_{H1}) of the heavy chain, in addition to the heavy and light chain variable domains of the Fv fragment. A “F(ab′)₂” fragment includes two Fab fragments joined, near the hinge region, by disulfide bonds.

The terms “polypeptide” and “protein” are used interchangeably to refer to a polymer of amino acid residues and are not limited to a minimum length. Such polymers of amino acid residues can contain natural or non-natural amino acid residues, and include, but are not limited to, dimers, trimers, peptides, oligopeptides, and multimers of amino acid residues. Both full-length proteins and fragments thereof are encompassed by the definition. The terms also include post-expression modifications of the polypeptide, for example, glycosylation, sialylation, acetylation, phosphorylation, and the like. The term “polypeptide” also refers to a protein which includes modifications, such as deletions, additions, and substitutions (generally conservative in nature), to the native sequence, so long as the protein maintains the desired activity. The terms “polypeptide” and “protein” encompass ALPP and/or ALPPL2 antigen binding proteins, including antibodies, antibody fragments, or sequences that have deletions from, additions to, and/or substitutions of one or more amino acids of the antigen binding protein.

A “native sequence” or a “naturally-occurring” polypeptide comprises a polypeptide having the same amino acid sequence as a polypeptide found in nature. Thus, a native sequence polypeptide can have the amino acid sequence of naturally-occurring polypeptide from any mammal. Such native sequence polypeptide can be isolated from nature or can be produced by recombinant or synthetic means. The term “native sequence” polypeptide specifically encompasses naturally-occurring truncated or secreted forms of the polypeptide (e.g., an extracellular domain sequence), naturally-occurring variant forms (e.g., alternatively spliced forms) and naturally-occurring allelic variants of the polypeptide.

A polypeptide “variant” means a biologically active polypeptide (e.g., an antigen binding protein or antibody) having at least about 70%, 80%, or 90% amino acid sequence identity with the native or a reference sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some embodiments, a variant will have at least about 80% amino acid sequence identity. In some embodiments, a variant will have at least about 90% amino acid sequence identity. In some embodiments, a variant will have at least about 95% amino acid sequence identity with the native sequence polypeptide.

As used herein, “Percent (%) amino acid sequence identity” and “homology” with respect to a peptide, polypeptide or antigen binding protein (e.g., antibody) sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared. For example, the % sequence identity of a given amino acid sequence A to, with, or against a given amino acid sequence B (which can alternatively be phrased as a given amino acid sequence A that has or comprises a certain % sequence identity to, with, or against a given amino acid sequence B) is calculated as follows:

100 times the fraction X/Y
where X is the number of amino acid residues scored as identical matches by the sequence in that program's alignment of A and B, and where Y is the total number of amino acid residues in B. Unless specifically stated otherwise, all % amino acid sequence identity values used herein are calculated according to this formula using the ALIGN-2 computer program. It will be appreciated that where the length of amino acid sequence A is not equal to the length of amino acid sequence B, the % sequence identity of A to B will not equal the % sequence identity of B to A.

The term “leader sequence” refers to a sequence of amino acid residues located at the N-terminus of a polypeptide that facilitates secretion of a polypeptide from a mammalian cell. A leader sequence may be cleaved upon export of the polypeptide from the mammalian cell, forming a mature protein. Leader sequences can be natural or synthetic, and they can be heterologous or homologous to the protein to which they are attached.

The term “immunoglobulin” refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See, for instance, Fundamental Immunology (Paul, W., ed., 7^{th} ed. Raven Press, N.Y. (2013)). Briefly, each heavy chain typically is comprised of a heavy chain variable region (abbreviated herein as V_{H} or VH) and a heavy chain constant region (C_{H} or CH). The heavy chain constant region typically is comprised of three domains, C_{H}1, C_{H}2, and C_{H}3. The heavy chains are generally inter-connected via disulfide bonds in the so-called “hinge region.” Each light chain typically is comprised of a light chain variable region (abbreviated herein as V_{L} or VL) and a light chain constant region (C_{L} or CL). The light chain constant region typically is comprised of one domain, C_{L}. The CL can be of κ (kappa) or λ (lambda) isotype. The terms “constant domain” and “constant region” are used interchangeably herein. An immunoglobulin can derive from any of the commonly known isotypes, including but not limited to IgA, secretory IgA, IgG, and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes.

The term “antibody,” is used in its broadest sense and specifically covers, for example, monoclonal antibodies (including full length or intact monoclonal antibodies), antibodies with polyepitopic or monoepitopic specificity, polyclonal or monovalent antibodies, multivalent antibodies, multispecific antibodies (e.g., bispecific antibodies so long as they exhibit the desired biological activity), single chain antibodies, and fragments of the foregoing, as described below. An antibody can be human, humanized, chimeric and/or affinity matured, as well as an antibody from other species, for example, mouse and rabbit, etc. The term “antibody” thus includes, for instance, a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). The term “antibody” also includes, but is not limited to, synthetic antibodies, recombinantly produced antibodies, camelized antibodies, intrabodies, anti-idiotypic (anti-Id) antibodies, and functional fragments (e.g., antigen-binding fragments) of any of the above, which refers to a portion of an antibody heavy and/or light chain polypeptide that retains some or all of the binding activity of the antibody from which the fragment was derived. Non-limiting examples of functional fragments (e.g., antigen-binding fragments) include single-chain Fvs (scFv) (e.g., including monospecific, bispecific, etc.), Fab fragments, F(ab′) fragments, F(ab)₂ fragments, F(ab′)₂ fragments, disulfide-linked Fvs (dsFv), Fd fragments, Fv fragments, diabody, triabodies, tetrabodies, and minibodies. In particular, antibodies provided herein include immunoglobulin molecules and immunologically active portions of immunoglobulin molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Pluckthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule.

The term “hypervariable region” or “HVR,” as used herein, refers to each of the regions of an antibody variable domain which are hypervariable in sequence. HVRs can form structurally defined loops (“hypervariable loops”). Generally, native four-chain antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, NJ, 2003). Indeed, naturally-occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

HVRs generally comprise amino acid residues from the hypervariable loops and/or from the “complementary determining regions” (CDRs), CDRs being of highest sequence variability and/or involved in antigen recognition. A variety of schemes for defining the boundaries of a given CDR are known in the art. For example, the Kabat Complementarity Determining Regions (CDRs) are based on sequence variability and are the most commonly used (Kabat et at., Sequences of Proteins of Immunological Interest, 5th Ed. Public Health Service, National Institutes of Health, Bethesda, MD. (1991)). Chothia refers instead to the location of the structural loops (Chothia and Lesk, J. Mol. Biol. 196:901-917 (1987)). The AbM CDRs represent a compromise between the Kabat CDRs and Chothia structural loops and are used by Oxford Molecular's AbM antibody modeling software. The “contact” CDRs are based on an analysis of the available complex crystal structures. Additional details on the foregoing schemes as well as other numbering conventions are provided in the following references: Al-Lazikani et al., (1997) J. Mol. Biol. 273: 927-948 (“Chothia” numbering scheme); MacCallum et al., (1996) J. Mol. Biol. 262:732-745 (1996), (Contact” numbering scheme); Lefranc M-P., et al., (2003) Dev. Comp. Immunol. 27:55-77 (“IMGT” numbering scheme); and Honegger A. & Pluckthun A. (2001) J. Mol/Biol. 309:657-70, (AHo numbering scheme).

In some embodiments, the HVR regions and associated sequences are the same as the CDR regions and associated sequences based upon one of the foregoing numbering conventions. As such, residues for exemplary HVRs and/or CDRs are summarized in Table 1 below.

TABLE 1
Summary of Different CDR Numbering Schemes

| Loop | IMGT | Kabat | AbM | Chothia | Contact |
|---|---|---|---|---|---|
| CDR-H1 | 27-38 | 31-35 | 26-35 | 26-32 | 30-35 |
| CDR-H2 | 56-65 | 50-65 | 50-58 | 52-56 | 47-58 |
| CDR-H3 | 105-117 | 95-102 | 95-102 | 95-102 | 93-101 |
| CDR-L1 | 27-38 | 24-34 | 24-34 | 24-34 | 30-36 |
| CDR-L2 | 56-65 | 50-56 | 50-56 | 50-56 | 46-55 |
| CDR-L3 | 105-117 | 89-97 | 89-97 | 89-97 | 89-96 |

In some embodiments, HVRs can comprise extended HVRs as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et at., supra, for each of these definitions.

Unless otherwise specified, the terms “CDR” and “complementary determining region” of a given antibody or region thereof, such as a variable region, as well as individual CDRs (e.g., “CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the IMGT, Kabat, AbM, Chothia, or Contact method. In other instances, the particular amino acid sequence of a CDR is given.

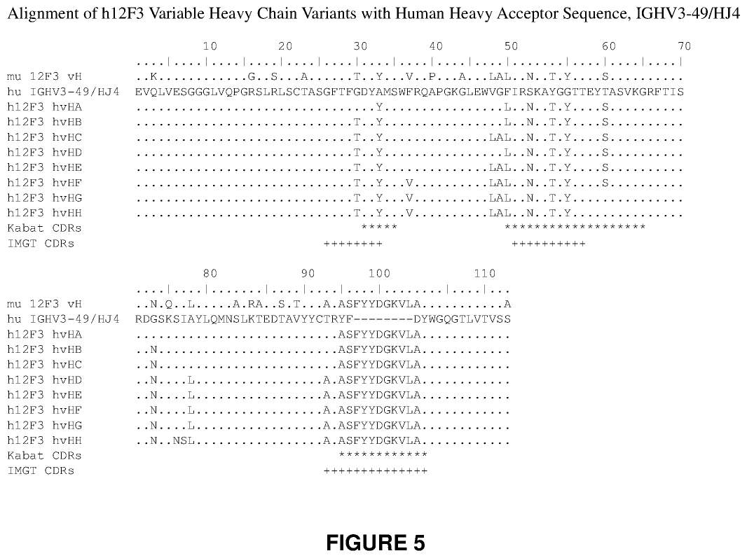

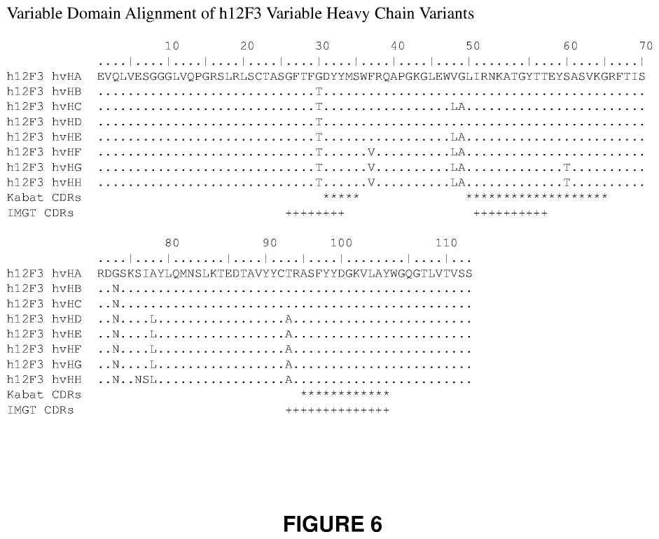

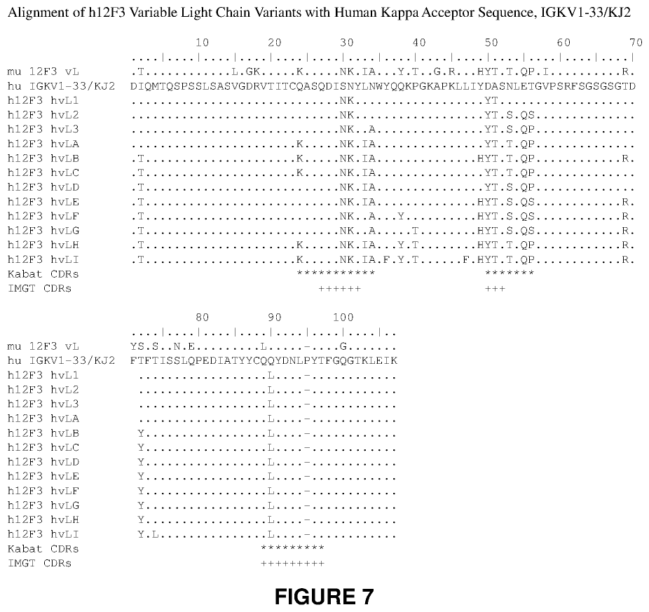

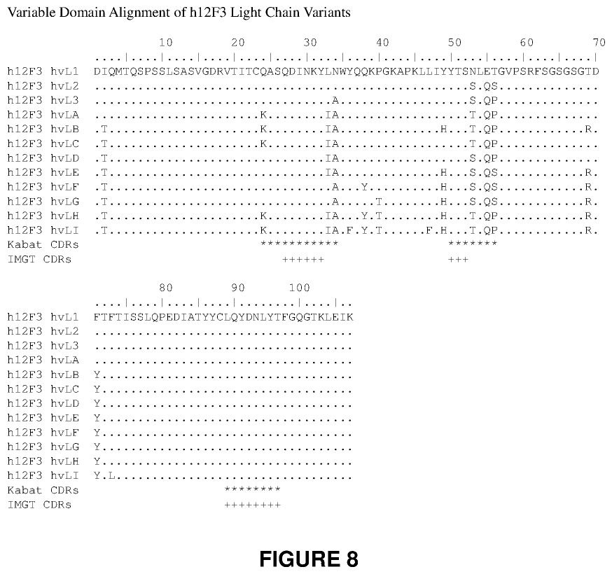

Thus, in some embodiments, the antigen binding protein comprises CDRs and/or HVRs as defined by the IMGT system. In other embodiments, the antigen binding protein comprises CDRs or HVRs as defined by the Kabat system. In still other embodiments, the antigen binding protein comprises CDRs or HVRs as defined by the AbM system. In further embodiments, the antigen binding protein comprises CDRs or HVRs as defined by the Chothia system. In some embodiments, the antigen binding proteins comprise the HVR and/or CDR residues as identified in FIGS. 5-8 or as set forth elsewhere herein.

The term “variable region” or “variable domain” refers to the domain of an antigen binding protein (e.g., an antibody) heavy or light chain that is involved in binding the antigen binding protein (e.g., antibody) to antigen. The variable regions or domains of the heavy chain and light chain (VH and VL, respectively) of an antigen binding protein such as an antibody can be further subdivided into regions of hypervariability (or hypervariable regions, which may be hypervariable in sequence and/or form of structurally defined loops), such as hypervariable regions (HVRs) or complementarity-determining regions (CDRs), interspersed with regions that are more conserved, termed framework regions (FRs). In general, there are three HVRs (HVR-H1, HVR-H2, HVR-H3) or CDRs (CDR-H1, CDR-H2, CDR-H3) in each heavy chain variable region, and three HVRs (HVR-L1, HVR-L2, HVR-L3) or CDRs in (CDR-L1, CDR-L2, CDR-L3) in each light chain variable region. “Framework regions” and “FR” are known in the art to refer to the non-HVR or non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). Within each VH and VL, three HVRs or CDRs and four FRs are typically arranged from amino-terminus to carboxy-terminus in the following order: FR1, HVR1, FR2, HVR2, FR3, HVR3, FR4 in the case of HVRs, or FR1, CDR1, FR2, CDR2, FR3, CDR3, FR4 in the case of CDRs (See also Chothia and Lesk J. Mot. Biol., 195, 901-917 (1987)). A single VH or VL domain can be sufficient to confer antigen-binding specificity. In addition, antibodies that bind a particular antigen can be isolated using a VH or VL domain from an antibody that binds the antigen to screen a library of complementary VL or VH domains, respectively. See, e.g., Portolano et al. J. Immunol. 150:880-887 (1993); Clarkson et al., Nature 352:624-628 (1991).

The term “heavy chain variable region” (VH) as used herein refers to a region comprising heavy chain HVR-H1, FR-H2, HVR-H2, FR-H3, and HVR-H3. For example, a heavy chain variable region may comprise heavy chain CDR-H1, FR-H2, CDR-H2, FR-H3, and CDR-H3. In some embodiments, a heavy chain variable region also comprises at least a portion of an FR-H1 and/or at least a portion of an FR-H4.

The term “heavy chain constant region” as used herein refers to a region comprising at least three heavy chain constant domains, C_{H}1, C_{H}2, and C_{H}3. Nonlimiting exemplary heavy chain constant regions include γ, δ, and α. Nonlimiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising a μ constant region is an IgM antibody, and an antibody comprising an E constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an α₁ constant region) and IgA2 (comprising an α₂ constant region) antibodies; and IgM antibodies include, but are not limited to, IgM1 and IgM2.

The term “heavy chain” (HC) as used herein refers to a polypeptide comprising at least a heavy chain variable region, with or without a leader sequence. In some embodiments, a heavy chain comprises at least a portion of a heavy chain constant region. The term “full-length heavy chain” as used herein refers to a polypeptide comprising a heavy chain variable region and a heavy chain constant region, with or without a leader sequence.

The term “light chain variable region” (VL) as used herein refers to a region comprising light chain HVR-L1, FR-L2, HVR-L2, FR-L3, and HVR-L3. In some embodiments, the light chain variable region comprises light chain CDR-L1, FR-L2, CDR-L2, FR-L3, and CDR-L3. In some embodiments, a light chain variable region also comprises an FR-L1 and/or an FR-L4.

The term “light chain constant region” as used herein refers to a region comprising a light chain constant domain, C_{L}. Nonlimiting exemplary light chain constant regions include λ and κ.

The term “light chain” (LC) as used herein refers to a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

The “EU numbering system” or “EU index” is generally used when referring to a residue in an immunoglobulin heavy chain constant region (e.g., the EU index reported in Kabat et al., Sequences of Proteins of Immunological Interest, 5th Ed. Public Health Service, National Institutes of Health, Bethesda, Md., 1991). The “EU index as in Kabat” refers to the residue numbering of the human IgG1 EU antibody. Unless stated otherwise herein, references to residue numbers in the constant domain of antibodies means residue numbering by the EU numbering system.

The term “monoclonal antibody” refers to an antibody obtained from a population of substantially homogeneous antibodies, that is, the individual antibodies comprising the population are identical except for possible naturally occurring mutations that can be present in minor amounts. Monoclonal antibodies are highly specific, being directed against a single antigenic site. In contrast to polyclonal antibody preparations, which can include different antibodies directed against different determinants (epitopes), each monoclonal antibody is directed against a single determinant on the antigen.

A “bispecific” antibody as used herein refers to an antibody, having binding specificities for at least two different antigenic epitopes. In one embodiment, the epitopes are from the same antigen. In another embodiment, the epitopes are from two different antigens. Methods for making bispecific antibodies are known in the art. For example, bispecific antibodies can be produced recombinantly using the co-expression of two immunoglobulin heavy chain/light chain pairs. See, e.g., Milstein et al., Nature 305:537-39 (1983). Alternatively, bispecific antibodies can be prepared using chemical linkage. See, e.g., Brennan, et al., Science 229:81 (1985). Bispecific antibodies include bispecific antibody fragments. See, e.g., Hollinger, et al., Proc. Natl. Acad. Sci. U.S.A. 90:6444-48 (1993), Gruber, et al., J. Immunol. 152:5368 (1994).

A “dual variable domain immunoglobulin” or “DVD-Ig” refers to multivalent and multispecific binding proteins as described, e.g., in DiGiammarino et al., Methods Mol. Biol. 899:145-156, 2012; Jakob et al., MABs 5:358-363, 2013; and U.S. Pat. Nos. 7,612,181; 8,258,268; 8,586,714; 8,716,450; 8,722,855; 8,735,546; and 8,822,645, each of which is incorporated by reference in its entirety.

A “dual-affinity re-targeting protein” or a “DART” is a form of a bispecific antibody in which the heavy variable domain from one antibody is linked with the light variable domain of another, and the two chains associate, and are described in, e.g., Garber, Nature Reviews Drug Discovery 13:799-801, 2014.

A “Bispecific T-cell Engager” or BiTE®”, is the genetic fusion of two scFv fragments resulting in tandem scFv molecules, and are described, e.g., in Baeuerle et al., Cancer Res. 69: 4941-4944, 2009.

A “chimeric antibody” as used herein refers to an antibody in which a portion of the heavy and/or light chain is derived from a particular source or species, while the remainder of the heavy and/or light chain is derived from a different source or species. In some embodiments, a chimeric antibody refers to an antibody comprising at least one variable region from a first species (such as mouse, rat, cynomolgus monkey, etc.) and at least one constant region from a second species (such as human, cynomolgus monkey, etc.). In some embodiments, a chimeric antibody comprises at least one mouse variable region and at least one human constant region. In some embodiments, a chimeric antibody comprises at least one cynomolgus variable region and at least one human constant region. In some embodiments, all of the variable regions of a chimeric antibody are from a first species and all of the constant regions of the chimeric antibody are from a second species.

The term “humanized antibody” as used herein, refers to a genetically engineered non-human antibody, which contains human antibody constant domains and non-human variable domains modified to contain a high level of sequence homology to human variable domains. This can be achieved by grafting of the six non-human antibody complementarity-determining regions (CDRs), onto a homologous human acceptor framework region (FR) (see WO92/22653 and EP0629240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

A “human antibody” as used herein refers to antibodies produced in humans, antibodies produced in non-human animals that comprise human immunoglobulin genes, such as XenoMouse®, and antibodies selected using in vitro methods, such as phage display, wherein the antibody repertoire is based on a human immunoglobulin sequence. A “human antibody” is one having variable regions in which both the FRs and CDRs are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibodies” and “fully human antibodies” and are used synonymously.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework derived from a human immunoglobulin framework or a human consensus framework can comprise the same amino acid sequence thereof, or it can contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

An “affinity matured” antibody refers to an antibody with one or more alterations in one or more hypervariable regions (HVRs) compared to a parent antibody which does not possess such alterations, such alterations resulting in an improvement in the affinity of the antibody for antigen. In some examples, an affinity matured antibody refers to an antibody with one or more alterations in one or more complementarity determining regions (CDRs) compared to a parent antibody which does not possess such alterations, such alterations resulting in an improvement in the affinity of the antibody for antigen.

The term “derivative” refers to a molecule (e.g., an antigen binding protein such as an antibody or fragment thereof) that includes a chemical modification other than an insertion, deletion, or substitution of amino acids (or nucleic acids). In certain embodiments, derivatives comprise covalent modifications, including, but not limited to, chemical bonding with polymers, lipids, or other organic or inorganic moieties. In certain embodiments, a derivative of a particular antigen binding protein can have a greater circulating half-life than an antigen binding protein that is not chemically modified. In certain embodiments, a derivative can have improved targeting capacity for desired cells, tissues, and/or organs. In some embodiments, a derivative of an antigen binding protein is covalently modified to include one or more polymers, including, but not limited to, monomethoxy-polyethylene glycol, dextran, cellulose, or other carbohydrate based polymers, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of such polymers. See, e.g., U.S. Pat. Nos. 4,640,835, 4,496,689, 4,301,144, 4,670,417, 4,791,192 and 4,179,337.

As used herein, the term “epitope” refers to a site on an antigen (e.g., ALPP or ALPPL2), to which an antigen-binding protein (e.g., an antibody or fragments thereof) that targets that antigen binds. Epitopes often consist of a chemically active surface grouping of molecules such as amino acids, polypeptides, sugar side chains, phosphoryl or sulfonyl groups, and have specific three-dimensional structural characteristics as well as specific charge characteristics. Epitopes can be formed both from contiguous or noncontiguous amino acids of the antigen that are juxtaposed by tertiary folding. Epitopes formed from contiguous residues typically are retained on exposure to denaturing solvents, whereas epitopes formed by tertiary folding typically are lost on treatment with denaturing solvents. In certain embodiments, an epitope can include, but is not limited to, at least 3, at least 4, at least 5, at least 6, at least 7, amino acids in a unique spatial arrangement. In some embodiments, the epitope refers to 3-5, 4-6, or 8-10 amino acids in a unique spatial conformation. In further embodiments, an epitope is less than 20 amino acids in length, less than 15 amino acids or less than 12 amino acids, less than 10 amino acids, or less than 8 amino acids in length. In an embodiment an epitope of an anti-ALPP/ALPPL2 antibody of the invention comprises SEQ ID NO: 73 and/or SEQ ID NO: 74. The epitope can comprise amino acids residues directly involved in the binding (also called immunodominant component of the epitope) and other amino acid residues that are not directly involved in the binding, including amino acid residues that are effectively blocked or covered by the antigen binding molecule (i.e., the amino acids are within the footprint of the antigen binding molecule). Methods of determining spatial conformation of epitopes include, for example, x-ray crystallography, two-dimensional nuclear magnetic resonance, and HDX-MS (see, e.g., Epitope Mapping Protocols in Methods in Molecular Biology, Vol. 66, G. E. Morris, Ed. (1996)). Once a desired epitope of an antigen is determined, antigen binding proteins (e.g., antibodies or fragments thereof) to that epitope can be generated using established techniques. It is then possible to screen the resulting antigen binding proteins in competition assays to identify antigen binding proteins that bind the same or overlapping epitopes. Methods for binning antibodies based upon cross-competition studies are described in WO 03/48731.

A “nonlinear epitope” or “conformational epitope” comprises noncontiguous polypeptides, amino acids, and/or sugars within the antigenic protein to which an antibody specific to the epitope binds.

A “linear epitope” comprises contiguous polypeptides, amino acids, and/or sugars within the antigenic protein to which an antigen binding protein (e.g., an antibody or fragment thereof) specific to the epitope binds.

The term “compete” when used in the context of antigen binding proteins (e.g., antibodies or fragments thereof) that compete for the same epitope means competition between antigen binding proteins as determined by an assay in which the antigen binding protein (e.g., an antibody or fragment thereof) being tested (e.g., a test antibody) prevents or inhibits (partially or completely) specific binding of a reference antigen binding protein (e.g., a reference antibody) to a common antigen (e.g., ALPP or ALPPL2 or a fragment thereof). Numerous types of competitive binding assays can be used to determine if one antigen binding protein competes with another, including various label-free biosensor approaches such as surface plasmon resonance (SPR) analysis (see, e.g., Abdiche, et al., 2009, Anal. Biochem. 386:172-180; Abdiche, et al., 2012, J Immunol Methods 382:101-116; and Abdiche, et al., 2014 PLoS One 9:e92451. Other assays that can be used include: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using I-125 label (see, e.g., Morel et al., 1988, Mol. Immunol. 25:7-15); solid phase direct biotin-avidin EIA (see, e.g., Cheung, et al., 1990, Virology 176:546-552); direct labeled RIA (Moldenhauer et al., 1990, Scand. J Immunol. 32:77-82). Typically, the test antigen binding protein is present in excess (e.g., at least 2×, 5×, 10×, 20× or 100×). Usually, when a competing antigen binding protein is present in excess, it will inhibit specific binding of a reference antigen binding protein to a common antigen by at least 40%, 50%, 60%, 70%, 80%, 90%, 95% or 100%. In instances in in which each antigen binding protein (e.g., an antibody or fragment thereof) detectably inhibits the binding of the other antigen binding protein with its cognate epitope, whether to the same, greater, or lesser extent, the antigen binding proteins are said to “cross-compete” with each other for binding of their respective epitope(s) or to “cross-block” one another. Typically, such cross-competition studies are done using the conditions and methods described above for competition studies and the extent of blocking is at least 30%, at least 40%, or at least 50% each way. Additional approaches and details on methods for identifying competing antigen binding proteins are described in the Examples herein.

“Affinity” refers to the strength of the sum total of noncovalent interactions between a single binding site of a molecule (e.g., an antibody) and its binding partner (e.g., an antigen). The affinity of a molecule X for its partner Y can generally be represented by the dissociation constant (K_{d}). Affinity can be measured by common methods known in the art, including those described herein.

An “affinity matured” antibody refers to an antibody with one or more alterations in one or more hypervariable regions (HVRs) compared to a parent antibody which does not possess such alterations, such alterations resulting in an improvement in the affinity of the antibody for antigen. In some examples, an affinity matured antibody refers to an antibody with one or more alterations in one or more complementarity determining regions (CDRs) compared to a parent antibody which does not possess such alterations, such alterations resulting in an improvement in the affinity of the antibody for antigen.

As used herein, the term “specifically binds”, “binding” or simply “binds” or other related terms in the context of the binding of an antigen binding protein to its target antigen means that the antigen binding protein exhibits essentially background binding to non-target molecules. An antigen binding protein that specifically binds the target antigen (e.g., ALPP and/or ALPPL2) may, however, cross-react with ALPP and/or ALPPL2 proteins from different species. Typically, a ALPP/ALPPL2 antigen binding protein specifically binds human ALPP and/or ALPPL2 when the dissociation constant (K_{D}) is 10⁻⁷ M or less, such as about 10⁻⁸ M or less, such as about 10⁻⁹ M or less, about 10⁻¹⁰ M or less, about 10⁻¹¹ M or less, or about 10⁻¹² or even less as measured via a surface plasma resonance (SPR) technique (e.g., BIACore, GE-Healthcare Uppsala, Sweden) using the antibody as the ligand and the antigen as the analyte.

The term “K_{D}” (M), as used herein, refers to the dissociation equilibrium constant of a particular antigen binding protein-antigen interaction (e.g., antibody-antigen interaction). Affinity, as used herein, and K_{D} are inversely related, such that higher affinity is intended to refer to lower K_{D}, and lower affinity is intended to refer to higher K_{D}.

An “antibody-drug-conjugate” or simply “ADC” refers to an antibody conjugated to a cytotoxic agent or cytostatic agent. An antibody-drug-conjugate typically binds to the target antigen (e.g., ALPP and/or ALPPL2) on a cell surface followed by internalization of the antibody-drug-conjugate into the cell where the drug is released.

The abbreviations “vc” and “val-cit” refer to the dipeptide valine-citrulline.

The abbreviation LAE refers to the tripeptide linker leucine-alanine-glutamic acid. The abbreviation dLAE refers to the tripeptide linker D-leucine-alanine-glutamic acid, where the leucine in the tripeptide linker is in the D-configuration.

The abbreviation VKG refers to the tripeptide linker valine-lysine-glycine.

The abbreviation “PABC” refers to the self-immolative spacer:

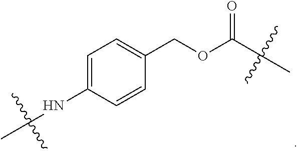 [SMILES: *Nc1ccc(COC(*)=O)cc1]

The abbreviation “mc” refers to the stretcher maleimidocaproyl:

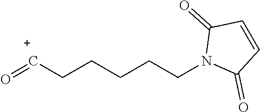 [SMILES: O=[C+]CCCCCN1C(=O)C=CC1=O]

The abbreviation “mp” refers to the stretcher maleimidopropionyl:

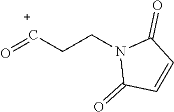 [SMILES: O=[C+]CCN1C(=O)C=CC1=O]

“PEG Unit” as used herein is an organic moiety comprised of repeating ethylene-oxy subunits (PEGs or PEG subunits) and may be polydisperse, monodisperse or discrete (i.e., having discrete number of ethylene-oxy subunits). Polydisperse PEGs are a heterogeneous mixture of sizes and molecular weights whereas monodisperse PEGs are typically purified from heterogeneous mixtures and are therefore provide a single chain length and molecular weight. Preferred PEG Units comprises discrete PEGs, compounds that are synthesized in step-wise fashion and not via a polymerization process. Discrete PEGs provide a single molecule with defined and specified chain length.

The PEG Unit provided herein comprises one or multiple polyethylene glycol chains, each comprised of one or more ethyleneoxy subunits, covalently attached to each other. The polyethylene glycol chains can be linked together, for example, in a linear, branched or star shaped configuration. Typically, at least one of the polyethylene glycol chains prior to incorporation into a camptothecin conjugate is derivatized at one end with an alkyl moiety substituted with an electrophilic group for covalent attachment to the carbamate nitrogen of a methylene carbamate unit (i.e., represents an instance of R). Typically, the terminal ethyleneoxy subunit in each polyethylene glycol chains not involved in covalent attachment to the remainder of the Linker Unit is modified with a PEG Capping Unit, typically an optionally substituted alkyl such as —CH₃, CH₂CH₃ or CH₂CH₂CO₂H. A preferred PEG Unit has a single polyethylene glycol chain with 2 to 24 —CH₂CH₂O— subunits covalently attached in series and terminated at one end with a PEG Capping Unit.

A “cytotoxic effect” refers to the depletion, elimination and/or killing of a target cell.

A “cytotoxic agent” refers to an agent that has a cytotoxic effect on a cell.

A “cytostatic effect” refers to the inhibition of cell proliferation.

A “cytostatic agent” refers to an agent that has a cytostatic effect on a cell, thereby inhibiting the growth of and/or expansion of a specific subset of cells. Cytostatic agents can be conjugated to an antibody or administered in combination with an antibody.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

A “functional Fc region” possesses an “effector function” of a native sequence Fc region. Exemplary “effector functions” include Fc receptor binding; C1q binding; complement dependent cytotoxicity (CDC); antibody-dependent cell-mediated cytotoxicity (ADCC); antibody-dependent cellular phagocytosis (ADCP); down regulation of cell surface receptors (e.g. B cell receptor; BCR), etc. Such effector functions generally require the Fc region to be combined with a binding domain (e.g., an antibody variable domain) and can be assessed using various assays.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification.

“Fc receptor” or “FcR” describes a receptor that binds to the Fc region of an antibody. In some embodiments, an FcγR is a native human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain Inhibiting receptor FcγRIIB contains an immunoreceptor tyrosine-based inhibition motif (ITIM) in its cytoplasmic domain. (see, e.g., Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward., Immunol. Today 18(12):592-598 (1997); Ghetie et al., Nature Biotechnology, 15(7):637-640 (1997); Hinton et al., J Biol. Chem. 279(8):6213-6216 (2004); WO 2004/92219 (Hinton et al.).

“Effector functions” refer to biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); antibody-dependent cellular phagocytosis (ADCP); down regulation of cell surface receptors (e.g. B cell receptor); and B cell activation. Such functions can be affected by, for example, binding of an Fc effector domain(s) to an Fc receptor on an immune cell with phagocytic or lytic activity or by binding of an Fc effector domain(s) to components of the complement system. Typically, the effect(s) mediated by the Fc-binding cells or complement components result in inhibition and/or depletion of the CD33 targeted cell. Fc regions of antibodies can recruit Fc receptor (FcR)-expressing cells and juxtapose them with antibody-coated target cells. Cells expressing surface FcR for IgGs including FcγRIII (CD16), FcγRII (CD32) and FcγRIII (CD64) can act as effector cells for the destruction of IgG-coated cells. Such effector cells include monocytes, macrophages, natural killer (NK) cells, neutrophils and eosinophils. Engagement of FcγR by IgG activates antibody-dependent cellular cytotoxicity (ADCC) or antibody-dependent cellular phagocytosis (ADCP). ADCC is mediated by CD16⁺ effector cells through the secretion of membrane pore-forming proteins and proteases, while phagocytosis is mediated by CD32⁺ and CD64⁺ effector cells (see, e.g., Fundamental Immunology, 4^{th} ed., Paul ed., Lippincott-Raven, N.Y., 1997, Chapters 3, 17 and 30; Uchida et al., 2004, J. Exp. Med. 199:1659-69; Akewanlop et al., 2001, Cancer Res. 61:4061-65; Watanabe et al., 1999, Breast Cancer Res. Treat. 53:199-207.

“Human effector cells” are leukocytes which express one or more FcRs and perform effector functions. In certain embodiments, the cells express at least FcγRIII and perform ADCC effector function(s). Examples of human leukocytes which mediate ADCC include peripheral blood mononuclear cells (PBMC), natural killer (NK) cells, monocytes, cytotoxic T cells, and neutrophils. The effector cells may be isolated from a native source, e.g., from blood.

“Antibody-dependent cell-mediated cytotoxicity” or “ADCC” refers to a mechanism of cytotoxicity in which the Fc region of antibodies bound to antigen on the cell surface of target cells interact with Fc receptors (FcRs) present on certain cytotoxic effector cells (e.g. NK cells, neutrophils, and macrophages). This interaction enables these cytotoxic effector cells to subsequently kill the target cell with cytotoxins. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 or 6,737,056 (Presta), can be performed. Useful effector cells for such assays include PBMC and NK cells. ADCC activity of the molecule of interest can also be assessed in vivo, e.g., in an animal model such as that disclosed in Clynes et al. Proc. Natl. Acad. Sci. (USA) 95:652-656 (1998). Additional polypeptide variants with altered Fc region amino acid sequences (polypeptides with a variant Fc region) and increased or decreased ADCC activity are described, e.g., in U.S. Pat. Nos. 7,923,538, and 7,994,290.

“Complement dependent cytotoxicity” or “CDC” refers to the lysis of a target cell in the presence of complement. Activation of the classical complement pathway is initiated by the binding of the first component of the complement system (C1q) to the Fc region of antibodies (of the appropriate subclass), which are bound to their cognate antigen on a target cell. This binding activates a series of enzymatic reactions culminating in the formation of holes in the target cell membrane and subsequent cell death. Activation of complement may also result in deposition of complement components on the target cell surface that facilitate ADCC by binding complement receptors (e.g., CR3) on leukocytes. To assess complement activation, a CDC assay, e.g., as described in Gazzano-Santoro et al., J. Immunol. Methods 202:163 (1996), can be performed. Polypeptide variants with altered Fc region amino acid sequences (polypeptides such as an antibody with a variant Fc region) and increased or decreased C1q binding capability are described, e.g., in U.S. Pat. No. 6,194,551 B1, U.S. Pat. Nos. 7,923,538, 7,994,290 and WO 1999/51642. See also, e.g., Idusogie et al., J. Immunol. 164: 4178-4184 (2000).

The term “antibody-dependent cellular phagocytosis”, or simply “ADCP”, refers to the process by which antibody-coated cells are internalized, either in whole or in part, by phagocytic immune cells (e.g., macrophages, neutrophils and dendritic cells) that bind to an Fc region of Ig.

A polypeptide variant with “altered” FcR binding affinity or ADCC activity (e.g., an antibody) is one which has either enhanced or diminished FcR binding activity and/or ADCC activity compared to a parent polypeptide or to a polypeptide comprising a native sequence Fc region. The polypeptide variant which “displays increased binding” to an FcR binds at least one FcR with better affinity than the parent polypeptide. The polypeptide variant which “displays decreased binding” to an FcR, binds at least one FcR with lower affinity than a parent polypeptide. In some embodiments, such variants which display decreased binding to an FcR may possess little or no appreciable binding to an FcR, e.g., 0-20% binding to the FcR compared to a native sequence IgG Fc region.

The terms “nucleic acid molecule”, “nucleic acid” and “polynucleotide” are used interchangeably herein and refer to a polymer of nucleotides of any length. Such polymers of nucleotides can contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

The term “vector” means any molecule or entity (e.g., nucleic acid, plasmid, bacteriophage or virus) used to transfer a nucleic acid molecule into a host cell. A vector typically includes a nucleic acid molecule engineered to contain a cloned polynucleotide or polynucleotides encoding a polypeptide or polypeptides of interest that can be propagated in a host cell. Examples of vectors include, but are not limited to, plasmids, viral vectors, and expression vectors, for example, recombinant expression vectors. A vector may include one or more of the following elements: an origin of replication, one or more regulatory sequences (such as, for example, promoters and/or enhancers) that regulate the expression of the polypeptide of interest, and/or one or more selectable marker genes. The term includes vectors which are self-replicating nucleic acid molecules as well as vectors incorporated into the genome of a host cell into which it has been introduced.

The term “expression vector” refers to a vector that is suitable for transformation of a host cell and that can be used to express a polypeptide of interest in a host cell.

The terms “host cell” or “host cell line” are used interchangeably herein and refer to a cell or population of cells that may be or has been a recipient of a vector or isolated polynucleotide. Host cells can be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6® cells (Crucell), and 293 and CHO cells, and their derivatives, such as 293-6E and DG44 cells, respectively. Such terms refer not only to the original cell, but also to the progeny of such a cell. Certain modifications may occur in succeeding generations due to, for example, mutation or environmental influences. Such progeny are also encompassed by the terms so long as the cells have the same function or biological activity as the original cells.

The term “control sequence” refers to a polynucleotide sequence that can affect the expression and processing of coding sequences to which it is ligated. The nature of such control sequences can depend upon the host organism. In particular embodiments, control sequences for prokaryotes can include a promoter, a ribosomal binding site, and a transcription termination sequence. Control sequences for eukaryotes can include, for example, promoters comprising one or a plurality of recognition sites for transcription factors, transcription enhancer sequences, and transcription termination sequence. “Control sequences” can include leader sequences and/or fusion partner sequences.

As used herein, “operably linked” means that the components to which the term is applied are in a relationship that allows them to carry out their inherent functions under suitable conditions. For example, a control sequence in a vector that is “operably linked” to a protein coding sequence is ligated thereto such that expression of the protein coding sequence is achieved under conditions compatible with the transcriptional activity of the control sequences. In the case in which two encoding sequences are operably linked, the phrase means that the two DNA fragments or encoding sequences are joined such that the amino acid sequences encoded by the two fragments remain in-frame.

The term “transfection” means the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al., 1981, Gene 13:197. Such techniques can be used to introduce one or more exogenous DNA moieties into suitable host cells.

The term “transformation” refers to a change in a cell's genetic characteristics, and a cell has been transformed when it has been modified to contain new DNA or RNA. For example, a cell is transformed where it is genetically modified from its native state by introducing new genetic material via transfection, transduction, or other techniques. Following transfection or transduction, the transforming DNA can recombine with that of the cell by physically integrating into a chromosome of the cell, or can be maintained transiently as an episomal element without being replicated, or can replicate independently as a plasmid. A cell is considered to have been “stably transformed” when the transforming DNA is replicated with the division of the cell.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, e.g., in the case of an RNA polynucleotide. Thus, a DNA polynucleotide that is contained in a vector inside a host cell may be referred to as “isolated”.

The terms “individual”, “subject”, or patient are used interchangeably herein to refer to an animal, for example a mammal. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some instances, the “individual” or “subject” is a human. In some examples, an “individual” or “subject” refers to an individual or subject (e.g., a human) in need of treatment for a disease or disorder.

A “disease” or “disorder” as used herein refers to a condition where treatment is needed.

“Cancer” and “tumor,” as used herein, are interchangeable terms that refer to any abnormal cell or tissue growth or proliferation in an animal. As used herein, the terms “cancer” and “tumor” encompass solid and hematological/lymphatic cancers and also encompass malignant, pre-malignant, and benign growth, such as dysplasia. A solid tumor is an abnormal growth or mass of tissue that usually does not contain cysts or liquid areas. Examples of cancer include but are not limited to, carcinoma, lymphoma, blastoma, sarcoma, and leukemia. More particular non-limiting examples of such cancers include squamous cell cancer, small-cell lung cancer, pituitary cancer, esophageal cancer, astrocytoma, soft tissue sarcoma, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, renal cancer, liver cancer, prostate cancer, vulval cancer, thyroid cancer, hepatic carcinoma, brain cancer, endometrial cancer, testis cancer, cholangiocarcinoma, gallbladder carcinoma, gastric cancer, melanoma, and various types of head and neck cancer.

“Tumor burden” also referred to as “tumor load,” refers to the total amount of tumor material distributed throughout the body. Tumor burden refers to the total number of cancer cells or the total size of tumor(s) throughout the body, including lymph nodes and bone narrow. Tumor burden can be determined by a variety of methods known in the art, such as, e.g., by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The terms “metastatic cancer” and “metastatic disease” mean cancers that have spread from the site of origin to another part of the body, e.g., to regional lymph nodes or to distant sites.

The terms “advanced cancer”, “locally advanced cancer”, “advanced disease” and “locally advanced disease” mean cancers that have extended through the relevant tissue capsule. Surgery is typically not recommended for patients with locally advanced disease, and these patients have substantially less favorable outcomes compared to patients having clinically localized (organ-confined) cancer.

As used herein, “treatment” is an approach for obtaining beneficial or desired clinical results. “Treatment” as used herein, covers any administration or application of a therapeutic for disease in a mammal, including a human. Beneficial or desired clinical results include, but are not limited to, any one or more of: alleviation of one or more symptoms, diminishment of extent of disease, preventing or delaying spread (e.g., metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total). Also encompassed by “treatment” is a reduction of pathological consequence of a proliferative disease.

In the context of cancer, the term “treating” includes any or all of: inhibiting growth of cancer cells, inhibiting replication of cancer cells, reducing the number of cancer cells, reducing the rate of cancer cell infiltration into peripheral organs, reducing the rate or extent of tumor metastasis, lessening of overall tumor burden, and ameliorating one or more symptoms associated with the cancer.

In the context of an autoimmune disease, the term “treating” includes any or all of: preventing replication of cells associated with an autoimmune disease state including, but not limited to, cells capable of producing an autoimmune antibody, lessening the autoimmune-antibody burden and ameliorating one or more symptoms of an autoimmune disease.

In the context of an infectious disease, the term “treating” includes any or all of preventing the growth, multiplication or replication of the pathogen that causes the infectious disease and ameliorating one or more symptoms of an infectious disease.

The terms “inhibition” or “inhibit” refer to a decrease or cessation of any phenotypic characteristic or to the decrease or cessation in the incidence, degree, or likelihood of that characteristic. To “reduce” or “inhibit” is to decrease, reduce or arrest an activity, function, and/or amount as compared to a reference. In certain embodiments, by “reduce” or “inhibit” is meant the ability to cause an overall decrease of 20% or greater. In another embodiment, by “reduce” or “inhibit” is meant the ability to cause an overall decrease of 50% or greater. In yet another embodiment, by “reduce” or “inhibit” is meant the ability to cause an overall decrease of 75%, 85%, 90%, 95%, or greater.

A “reference” as used herein, refers to any sample, standard, or level that is used for comparison purposes. A reference can be obtained from a healthy and/or non-diseased sample. In some examples, a reference can be obtained from an untreated sample. In some examples, a reference is obtained from a non-diseased on non-treated sample of a subject individual. In some examples, a reference is obtained from one or more healthy individuals who are not the subject or patient.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease.

As used herein, to “suppress” a function or activity is to reduce the function or activity when compared to otherwise same conditions except for a condition or parameter of interest, or alternatively, as compared to another condition. For example, an antibody which suppresses tumor growth reduces the rate of growth of the tumor compared to the rate of growth of the tumor in the absence of the antibody.

An “effective amount” or “therapeutically effective amount” or “therapeutically effective dosage” of a drug or therapeutic agent is any amount of the drug or agent that, when used alone or in combination with another therapeutic agent provides a treatment effect, such as protecting a subject against the onset of a disease or promoting disease regression as evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example for the treatment of tumors, in some embodiments a therapeutically effective amount of an anti-cancer agent inhibits cell growth or tumor growth by at least about 10%, by at least about 20%, by at least about 30%, by at least about 40%, by at least about 50%, by at least about 60%, by at least about 70%, or by at least about 80%, by at least about 90%, by at least about 95%, by at least about 96%, by at least about 97%, by at least about 98%, or by at least about 99% in a treated subject(s) (e.g., one or more treated subjects) relative to an untreated subject(s) (e.g., one or more untreated subjects). In some embodiments, a therapeutically effective amount of an anti-cancer agent inhibits cell growth or tumor growth by 100% in a treated subject(s) (e.g., one or more treated subjects) relative to an untreated subject(s) (e.g., one or more untreated subjects). In other embodiments of the disclosure, tumor regression can be observed and continue for a period of at least about 20 days, at least about 30 days, at least about 40 days, at least about 50 days, or at least about 60 days.

A therapeutically effective amount of a drug includes a “prophylactically effective amount,” which is any amount of the drug that, when administered alone or in combination with an anti-cancer agent to a subject at risk of developing a cancer (e.g., a subject having a pre-malignant condition) or of suffering a recurrence of cancer, inhibits the development or recurrence of the cancer. In some embodiments, the prophylactically effective amount prevents the development or recurrence of the cancer entirely. “Inhibiting” the development or recurrence of a cancer means either lessening the likelihood of the cancer's development or recurrence, or preventing the development or recurrence of the cancer entirely.

As used herein, “subtherapeutic dose” means a dose of a therapeutic compound that is lower than the usual or typical dose of the therapeutic compound when administered alone for the treatment of a hyperproliferative disease (e.g., cancer).

“Administering” or “administration” refer to the physical introduction of a therapeutic agent to a subject, using any of the various methods and delivery systems known to those skilled in the art. Exemplary routes of administration include intravenous, intramuscular, subcutaneous, intraperitoneal, spinal or other parenteral routes of administration, for example by injection or infusion (e.g., intravenous infusion). Administration can also be performed, for example, once, a plurality of times, and/or over one or more extended periods.

The term “monotherapy” as used herein means that the anti-ALPP/ALPPL2 antibody or ADC of the invention is the only anti-cancer agent administered to the subject during the treatment cycle. Other therapeutic agents, however, can be administered to the subject. For example, anti-inflammatory agents or other agents administered to a subject with cancer to treat symptoms associated with cancer, but not the underlying cancer itself, including, for example inflammation, pain, weight loss, and general malaise, can be administered during the period of monotherapy.

Administration “in combination with” one or more further therapeutic agents includes simultaneous (concurrent) and consecutive or sequential administration in any order.

The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered simultaneously or with a time separation of no more than about 60 minutes, such as no more than about any of 30, 15, 10, 5, or 1 minutes.

The term “sequentially” is used herein to refer to administration of two or more therapeutic agents where the administration of one or more agent(s) continues after discontinuing the administration of one or more other agent(s). For example, administration of the two or more therapeutic agents are administered with a time separation of more than about 15 minutes, such as about any of 20, 30, 40, 50, or 60 minutes, 1 day, 2 days, 3 days, 1 week, 2 weeks, or 1 month, or longer.

The term “chemotherapeutic agent” refers to all chemical compounds that are effective in inhibiting tumor growth. Non-limiting examples of chemotherapeutic agents include alkylating agents (e.g., nitrogen mustards, ethyleneimine compounds and alkyl sulphonates); antimetabolites (e.g., folic acid, purine or pyrimidine antagonists); mitotic inhibitors (e.g., anti-tubulin agents such as vinca alkaloids, auristatins and derivatives of podophyllotoxin); cytotoxic antibiotics; compounds that damage or interfere with DNA expression or replication (e.g., DNA minor groove binders); and growth factor receptor antagonists, and cytotoxic or cytostatic agents.

The phrase “pharmaceutically acceptable” indicates that the substance or composition is compatible chemically and/or toxicologically with the other ingredients comprising a formulation, and/or the subject being treated therewith.

The terms “pharmaceutical formulation” and “pharmaceutical composition” refer to a preparation which is in such form as to permit the biological activity of the active ingredient(s) to be effective, and which contains no additional components which are unacceptably toxic to a subject to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, pamoate (i.e., 4,4′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

Various aspects of the disclosure are described in further detail in the following sections.

### II. Overview

The invention provides antibody-drug conjugates, including anti-ALPP antibodies conjugated to vcMMAE (sometimes referred to herein as mc-vc-PABC-MMAE or me-vc-MMAE) or dLAE-MMAE (sometimes referred to herein as mp-dLAE-PABC-MMAE or mp-dLAE-MMAE), that are particularly effective at killing ALPP+ expressing cells. The invention also provides antibody-drug conjugates including anti-ALPPL2 antibodies conjugated to vcMMAE or dLAE-MM. E that are particularly effective at killing ALPPL2+ expressing cells. In preferred embodiments, the invention provides antibodies that bind both ALPP and ALPPL2 (anti-ALPP/ALPPL2 antibodies) conjugated to vcMMAE or dLAE-MMAE, that are particularly effective at killing both ALPP+ and ALPPL2+ expressing cells. Both ALPP and ALPPL2 have been shown to be expressed in a variety of cancers, including ovarian cancer, lung cancer, endometrial cancer, testicular cancer, and gastric cancer.

Antigen binding proteins (ABP), including antigen binding fragments thereof, (e.g., antibodies and antigen binding fragments thereof) that bind ALPP/ALPPL2 are provided herein. In some embodiments, the antigen binding proteins and fragments contain an antigen binding domain that specifically binds to ALPP, including to human ALPP (e.g., SEQ ID NO: 2). In some embodiments, the antigen binding proteins and fragments contain an antigen binding domain that specifically binds to ALPPL2, including to human ALPPL2 (e.g., SEQ ID NO: 4). In some embodiments, the antigen binding proteins and fragments contain an antigen binding domain that specifically binds both ALPP and ALPPL2, including to human ALPP (e.g., SEQ ID NO: 2) and human ALPPL2 (e.g., SEQ ID NO: 4).

### III. Anti-ALPP/ALPPL2 Antigen Binding Proteins, Including Fragments

A variety of antigen binding proteins are provided herein and are described in greater detail below. The antigen binding proteins that are disclosed herein typically comprise a scaffold, such as a polypeptide or polypeptides, into which one or more (e.g., 1, 2, 3, 4, 5 or 6) hypervariable regions (HVRs) or complementarity determining regions (CDRs) are embedded, grafted, and/or joined. In some antigen binding proteins, the HVRs or CDRs are embedded, grafted or joined into a “framework” region, which orients the HVRs or CDR(s) such that the proper antigen binding properties of the HVRs or CDRs are achieved. In some embodiments, the antigen binding protein comprises one or more VH and/or VL domains.

In some antigen binding proteins, the HVR or CDR sequences are embedded, grafted or joined in or into a protein scaffold or other biocompatible polymer. In some embodiments, the antigen binding protein is an antibody, or is derived from an antibody. Accordingly, the antigen binding proteins that are provided include, but are not limited to, monoclonal antibodies, multispecific antibodies (e.g., bispecific antibodies), minibodies, domain antibodies, synthetic antibodies (sometimes referred to herein as “antibody mimetics”), chimeric antibodies, humanized antibodies, human antibodies, antibody fusions, antibody conjugates, and portions or fragments of each of the foregoing. Examples of antigen binding proteins provided herein that are fragments include, but are not limited to, a Fab, a Fab′, a F(ab′)₂, a scFv, and a domain antibody.

The antigen binding proteins in some embodiments bind to ALPP with an affinity (e.g., K_{D}) of less than 10 nM, 5 nM, 2 nM, 1 nM, 500 pM, 250 pM, 200 pM, 150 pM, 130 pM, 100 pM, 50 pM, 25 pM, 10 pM, or 1 pM. In some embodiments, the ABP binds to ALPP with an affinity of between 1 pM-10 nM, 1 pM-5 nM, 1 pM-1 nM, 100-200 pM, 100-150 pM, or 120-140 pM. In some embodiments, the binding affinity to ALPP is determined according to the assays described in the Examples. The antigen binding proteins in some embodiments bind to ALPPL2 with an affinity (e.g., K_{D}) of less than 10 nM, 5 nM, 2 nM, 1 nM, 500 pM, 250 pM, 100 pM, 50 pM, 44 pM, 25 pM, 10 pM, or 1 pM. In some embodiments, the ABP binds to ALPPL2 with an affinity of between 0.1 pM-5 nM, 0.1 pM-1 nM, 1 pM-1 nM, 1-100 pM, 1-75 pM, 10-75 pM, 10-50 pM, or 30-50 pM. In some embodiments, the binding affinity to ALPPL2 is determined according to the assays described in the Examples.

### A. Exemplary Antigen Binding Proteins, Including Fragments

In one embodiment, the antigen binding protein of the invention includes the antibody 12F3, described in the Examples herein. In some embodiments, the antigen binding protein of the invention includes the murine, chimeric, humanized, and/or human 12F3 antibodies.

In an embodiment, an antigen binding protein as disclosed herein comprises a CDR-H1, a CDR-H2, a CDR-H3, comprising the amino acid sequences of SEQ ID NOs: 56-58 or 60-62, respectively; and a CDR-L1, a CDR-L2, and a CDR-L3, comprising the amino acid sequences of SEQ ID NOs: 63-65 or 68-70, respectively. In a further embodiment, an antigen binding protein as disclosed herein comprises a CDR-H1, a CDR-H2, a CDR-H3, comprising the amino acid sequences of SEQ ID NOs: 56-58, respectively, and a CDR-L1, a CDR-L2, and a CDR-L3, comprising the amino acid sequences of SEQ ID NOs: 63-65, respectively, wherein the CDRs are determined by Kabat. In a further embodiment, an antigen binding protein as disclosed herein comprises a CDR-H1, a CDR-H2, a CDR-H3, comprising the amino acid sequences of SEQ ID NOs: 60-62, respectively, and a CDR-L1, a CDR-L2, and a CDR-L3, comprising the amino acid sequences of SEQ ID NOs: 68-70, respectively, wherein the CDRs are determined by IMGT.

In a further embodiment, an antigen binding protein as disclosed herein comprises a VH comprising the amino acid sequence of SEQ ID NO: 15, and a VL comprising the amino acid sequence of SEQ ID NO: 30.

In a further embodiment, an antigen binding protein as disclosed herein comprises an HC comprising the amino acid sequence of SEQ ID NO: 40, and an LC comprising the amino acid sequence of SEQ ID NO: 50.

In other embodiments, the antigen binding proteins that are provided include or are derived from one or more of the CDRs, variable heavy chains, variable light chains, heavy chains, and/or light chains of the antibodies described below.

In still another embodiment, the ABP comprises (a) a VH domain comprising at least one, at least two, or all three VH CDR sequences, wherein the VH CDR sequence(s) is/are selected from (i) a CDR-H1 comprising the amino acid sequence SEQ ID NO: 56 or SEQ ID NO: 60; (ii) a CDR-H2 comprising the amino acid sequence SEQ ID NO: 57 or SEQ ID NO: 61; and (iii) a CDR-H3 comprising the amino acid sequence SEQ ID NO: 58 or SEQ ID NO: 62, and (b) a VL domain comprising at least one, at least two, or all three VL CDR sequences, wherein the VL CDR sequence(s) is/are selected from (i) a CDR-L1 comprising the amino acid sequence SEQ ID NO: 63 or SEQ ID NO: 68; (ii) a CDR-L2 comprising the amino acid sequence SEQ ID NO: 64 or SEQ ID NO: 69; and (iii) a CDR-L3 comprising the amino acid sequence SEQ ID NO: 65 or SEQ ID NO: 70, provided that in embodiments in which the ABP comprises multiple CDRs each CDR is selected from a different group.

In still another embodiment, the ABP comprises (a) a VH domain comprising at least one, at least two, or all three VH CDR sequences, wherein the VH CDR sequence(s) is/are selected from (i) a CDR-H1 comprising the amino acid sequence SEQ ID NO: 56; (ii) a CDR-H2 comprising the amino acid sequence SEQ ID NO: 57; and (iii) a CDR-H3 comprising the amino acid sequence SEQ ID NO: 58, and (b) a VL domain comprising at least one, at least two, or all three VL CDR sequences, wherein the VL CDR sequence(s) is/are selected from (i) a CDR-L1 comprising the amino acid sequence SEQ ID NO: 63; (ii) a CDR-L2 comprising the amino acid sequence SEQ ID NO: 64; and (iii) a CDR-L3 comprising the amino acid sequence SEQ ID NO: 65, wherein the CDRs are determined by Kabat, and provided that in embodiments in which the ABP comprises multiple CDRs each CDR is selected from a different group.

In still another embodiment, the ABP comprises (a) a VH domain comprising at least one, at least two, or all three VH CDR sequences, wherein the VH CDR sequence(s) is/are selected from (i) a CDR-H1 comprising the amino acid sequence SEQ ID NO: 60; (ii) a CDR-H2 comprising the amino acid sequence SEQ ID NO: 61; and (iii) a CDR-H3 comprising the amino acid sequence SEQ ID NO: 62, and (b) a VL domain comprising at least one, at least two, or all three VL CDR sequences, wherein the VL CDR sequence(s) is/are selected from (i) a CDR-L1 comprising the amino acid sequence SEQ ID NO: 68; (ii) a CDR-L2 comprising the amino acid sequence SEQ ID NO: 69; and (iii) a CDR-L3 comprising the amino acid sequence SEQ ID NO: 70, wherein the CDRs are determined by IMGT, and provided that in embodiments in which the ABP comprises multiple CDRs each CDR is selected from a different group.

In yet another embodiment, the ABP comprises (a) a CDR-H1 comprising the amino acid sequence SEQ ID NO: 56 or SEQ ID NO: 60; (b) a CDR-H2 comprising the amino acid sequence SEQ ID NO: 57 or SEQ ID NO: 61; and (c) a CDR-H3 comprising the amino acid sequence SEQ ID NO: 58 or SEQ ID NO: 62, (d) a CDR-L1 comprising the amino acid sequence SEQ ID NO: 63 or SEQ ID NO: 68 (e) a CDR-L2 comprising the amino acid sequence SEQ ID NO: 64 or SEQ ID NO: 69; and (f) a CDR-L3 comprising the amino acid sequence SEQ ID NO: 65 or SEQ ID NO: 70.

In yet another embodiment, the ABP comprises (a) a CDR-H1 comprising the amino acid sequence SEQ ID NO: 56; (b) a CDR-H2 comprising the amino acid sequence SEQ ID NO: 57; and (c) a CDR-H3 comprising the amino acid sequence SEQ ID NO: 58, (d) a CDR-L1 comprising the amino acid sequence SEQ ID NO: 63 (e) a CDR-L2 comprising the amino acid sequence SEQ ID NO: 64; and (f) a CDR-L3 comprising the amino acid sequence SEQ ID NO: 65; wherein the CDRs are determined by Kabat.

In yet another embodiment, the ABP comprises (a) a CDR-H1 comprising the amino acid sequence SEQ ID NO: 60; (b) a CDR-H2 comprising the amino acid sequence SEQ ID NO: 61; and (c) a CDR-H3 comprising the amino acid sequence SEQ ID NO: 62, (d) a CDR-L1 comprising the amino acid sequence SEQ ID NO: 68 (e) a CDR-L2 comprising the amino acid sequence SEQ ID NO: 69; and (f) a CDR-L3 comprising the amino acid sequence SEQ ID NO: 70; wherein the CDRs are determined by IMGT.

Certain ABPs comprise a VH comprising a CDR-H1, a CDR-H2, and a CDR-H3, wherein the CDRs of the VH collectively have at most 1, 2, 3, 4, or 5 amino acid changes relative to a corresponding CDR reference sequence, and wherein the CDR-H1 reference sequence has the amino acid sequence SEQ ID NO: 56 or SEQ ID NO: 60, the CDR-H2 reference sequence has the amino acid sequence SEQ ID NO: 57 or SEQ ID NO: 61, and the CDR-H3 reference sequence has the amino acid sequence SEQ ID NO: 58 or SEQ ID NO: 62. In such embodiments, the amino acid changes typically are insertions, deletions and/or substitutions. In some of these embodiments, the collective number of amino acid changes are 1-3; in other embodiments, the collective number of amino acid changes are 1 or 2. In certain of the foregoing embodiments, the changes are conservative amino acid substitutions.

In other embodiments, an ABP comprises a VL comprising a CDR-L1, a CDR-L2, and a CDR-L3, wherein the CDRs of the VL collectively have at most 1, 2, 3, 4, or 5 amino acid changes relative to a corresponding CDR reference sequence, and wherein the CDR-L1 reference sequence has the amino acid sequence SEQ ID NO: 63 or SEQ ID NO: 68, the CDR-L2 reference sequence has the amino acid sequence SEQ ID NO: 64 or SEQ ID NO: 69, and the CDR-L3 reference sequence has the amino acid sequence SEQ ID NO: 65 or SEQ ID NO: 70. In such embodiments, the amino acid changes typically are insertions, deletions and/or substitutions. In some of these embodiments, the collective number of amino acid changes are 1-3; in other embodiments, the collective number of amino acid changes are 1 or 2. In certain of the foregoing embodiments, the changes are conservative amino acid substitutions.

In a further embodiment, an ABP comprises (a) a VH comprising a CDR-H1, a CDR-H2, and a CDR-H3, wherein the CDRs of the VH collectively have at most 1, 2, 3, 4, or 5 amino acid changes relative to a corresponding CDR reference sequence, and wherein the CDR-H1 reference sequence has the amino acid sequence SEQ ID NO: 56 or SEQ ID NO: 60, the CDR-H2 reference sequence has the amino acid sequence SEQ ID NO: 57 or SEQ ID NO: 61, and the CDR-H3 reference sequence has the amino acid sequence SEQ ID NO: 58 or SEQ ID NO: 62, and (b) a VL comprising a CDR-L1, a CDR-L2, and a CDR-L3, wherein the CDRs of the VL collectively have at most 1, 2, 3, 4, or 5 amino acid changes relative to a corresponding CDR reference sequence, and wherein the CDR-L1 reference sequence has the amino acid sequence SEQ ID NO: 63 or SEQ ID NO: 68, the CDR-L2 reference sequence has the amino acid sequence SEQ ID NO: 64 or SEQ ID NO: 69, and the CDR-L3 reference sequence has the amino acid sequence SEQ ID NO: 65 or SEQ ID NO: 70. In such embodiments, the amino acid changes typically are insertions, deletions and/or substitutions. In some of these embodiments, the collective number of amino acid changes are 1-3; in other embodiments, the collective number of amino acid changes are 1 or 2. In certain of the foregoing embodiments, the changes are conservative amino acid substitutions.

The ABP in another embodiment comprises a VH domain, wherein the VH domain sequence has at least 80%, 85%, 90%, 91%, 92%, 93%, 94%, 95%, 96%, 97%, 98%, or 99% sequence identity to the amino acid sequence selected from any one of SEQ ID NOs: 9-16, provided the ABP retains the ability to bind to ALPP and/or ALPPL2. In certain embodiments, such an ABP contains substitutions (e.g., conservative substitutions), insertions, and/or deletions relative to the reference sequence (i.e., one of SEQ ID NOs: 9-16), provided that such an ABP retains the ability to bind to ALPP and/or ALPPL2. In certain embodiments, 1, 2, 3, 4, 5, 6, 7, 8, 9, or 10 amino acids have been substituted, inserted and/or deleted in any one of SEQ ID NOs: 9-16. In some embodiments, 1-5 or 1-3 amino acids have been substituted, inserted and/or deleted in the VH sequence. In certain of these embodiments, such substitutions, insertions, or deletions occur in regions outside the CDRs (i.e., in the FRs).

The ABP in another embodiment comprises a VL domain, wherein the VL domain sequence has at least 80%, 85%, 90%, 91%, 92%, 93%, 94%, 95%, 96%, 97%, 98%, or 99% sequence identity to the amino acid sequence selected from any one of SEQ ID NOs: 22-33, provided the ABP retains the ability to bind to ALPP and/or ALPPL2. In certain embodiments, such an ABP contains substitutions (e.g., conservative substitutions), insertions, and/or deletions relative to the reference sequence (i.e., one of SEQ ID NOs: 22-33), provided that such an ABP retains the ability to bind to ALPP and/or ALPPL2. In certain embodiments, 1, 2, 3, 4, 5, 6, 7, 8, 9, or 10 amino acids have been substituted, inserted and/or deleted in any one of SEQ ID NOs: 22-33. In some embodiments, 1-5 or 1-3 amino acids have been substituted, inserted and/or deleted in the VL sequence. In certain of these embodiments, such substitutions, insertions, or deletions occur in regions outside the CDRs (i.e., in the FRs).

In a further embodiment, the ABP comprises (a) a VH domain, wherein the VH domain sequence has at least 80%, 85%, 90%, 91%, 92%, 93%, 94%, 95%, 96%, 97%, 98%, or 99% sequence identity to the amino acid sequence selected from any one of SEQ ID NOs: 9-16, and (b) a VL domain, wherein the VL domain sequence has at least 80%, 85%, 90%, 91%, 92%, 93%, 94%, 95%, 96%, 97%, 98%, or 99% sequence identity to the amino acid sequence selected from any one of SEQ ID NOs: 22-33, provided the ABP retains the ability to bind to ALPP and/or ALPPL2.

In a further embodiment, the ABP comprises (a) a VH domain, wherein the VH domain sequence has at least 80%, 85%, 90%, 91%, 92%, 93%, 94%, 95%, 96%, 97%, 98%, or 99% sequence identity to the amino acid sequence SEQ ID NO: 15, and (b) a VL domain, wherein the VL domain sequence has at least 80%, 85%, 90%, 91%, 92%, 93%, 94%, 95%, 96%, 97%, 98%, or 99% sequence identity to the amino acid sequence SEQ ID NO: 30, provided the ABP retains the ability to bind to ALPP and/or ALPPL2.

The antigen binding protein in any of the foregoing embodiments can be an antibody in any form. As such, the antigen binding protein described in any of the above embodiments can be, for example, a monoclonal antibody, a multispecific antibody, a human, humanized or chimeric antibody, and ALPP binding fragments of any of the above, such as a single chain antibody, a Fab fragment, an F(ab′) fragment, or a fragment produced by a Fab expression library. The antibodies can be of any immunoglobulin isotype (e.g., IgG, IgE, IgM, IgD, IgA and IgY), class (e.g., IgG1, IgG2, IgG3, IgG4, IgA1 and IgA2) or subclass.

In certain embodiments, an ABP with the CDR and/or variable domain sequences described herein is an antigen-binding fragment (e.g., human antigen-binding fragments) and include, but are not limited to, Fab, Fab′ and F(ab′)2, Fd, single-chain Fvs (scFv), single-chain antibodies, disulfide-linked Fvs (sdFv) and fragments comprising either a VL or VH domain. Antigen-binding fragments, including single-chain antibodies, may comprise the variable region(s) alone or in combination with the entirety or a portion of the following: hinge region, CH1, CH2, CH3 and CL domains. Also included in the present disclosure are antigen-binding fragments comprising any combination of variable region(s) with a hinge region, CH1, CH2, CH3 and CL domains.

The ABP can be monospecific, bispecific, trispecific or of greater multi specificity. Multispecific antibodies can be specific for different epitopes of ALPP and/or ALPPL2 or may be specific for both ALPP and/or ALPPL2 as well as for a heterologous protein. See, e.g., PCT publications WO 93/17715; WO 92/08802; WO 91/00360; WO 92/05793; Tutt, et al., 1991, J. Immunol. 147:60 69; U.S. Pat. Nos. 4,474,893; 4,714,681; 4,925,648; 5,573,920; 5,601,819; and Kostelny et al., 1992, J. Immunol. 148:1547 1553.

In any of the embodiments described herein, one or several amino acids (e.g., 1, 2, 3 or 4) at the amino or carboxy terminus of the light and/or heavy chain, such as the C-terminal lysine of the heavy chain, may be missing or derivatized in some or all of the molecules in a composition. One specific example of such a modification, is an ABP in which the carboxy terminal lysine of the heavy chain is missing (e.g., as part of a post-translational modification). Furthermore, it should be understood that any of the sequences described herein include post-translational modifications to the specified sequence during expression of the ABP in cell culture (e.g., a CHO cell culture).

### B. Chimeric Antigen Binding Proteins

In certain embodiments, the antigen binding protein provided herein is a chimeric antibody. In some embodiments, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Certain chimeric antibodies are described, e.g., in U.S. Pat. No. 4,816,567; and Morrison et al., (1984) Proc. Natl. Acad. Sci. USA, 81:6851-6855 (1984)). Chimeric antibodies include antigen-binding fragments thereof.

Nonlimiting exemplary chimeric antibodies include chimeric antibodies comprising any of the heavy and/or light chain variable regions as described herein. In certain embodiments, the heavy and/or light chain variable domains are selected from Additional nonlimiting exemplary chimeric antibodies include chimeric antibodies comprising heavy chain HVR sequences (e.g., CDRs) or portions thereof, and/or light chain HVR sequences (e.g., CDRs) as provided herein.

### C. Humanized Antigen Binding Proteins

In certain embodiments, the ABP is a humanized antibody that binds ALPP and/or ALPPL2. Typically, a non-human antibody is humanized to reduce immunogenicity to humans, while retaining the specificity and affinity of the parental non-human antibody. A humanized antibody is a genetically engineered antibody in which the HVRs (e.g., CDRs) or portions thereof from a non-human “donor” antibody are grafted into human “acceptor” antibody sequences (see, e.g., Queen, U.S. Pat. Nos. 5,530,101 and 5,585,089; Winter, U.S. Pat. No. 5,225,539; Carter, U.S. Pat. No. 6,407,213; Adair, U.S. Pat. No. 5,859,205; and Foote, U.S. Pat. No. 6,881,557).

The acceptor antibody sequences can be, for example, a mature human antibody sequence, a composite of such sequences, a consensus sequence of human antibody sequences, or a germline region sequence. Human acceptor sequences can be selected for a high degree of sequence identity in the variable region frameworks with donor sequences to match canonical forms between acceptor and donor HVRs or CDRs among other criteria. Thus, a humanized antibody is an antibody having HVRs or CDRs entirely or substantially from a donor antibody and variable region framework sequences and constant regions, if present, entirely or substantially from human antibody sequences. Similarly, a humanized heavy chain typically has all three HVRs or CDRs entirely or substantially from a donor antibody heavy chain, and a heavy chain variable region framework sequence and heavy chain constant region, if present, substantially from human heavy chain variable region framework and constant region sequences. Likewise, a humanized light chain usually has all three CDRs entirely or substantially from a donor antibody light chain, and a light chain variable region framework sequence and light chain constant region, if present, substantially from human light chain variable region framework and constant region sequences. An HVR or CDR in a humanized antibody is substantially from a corresponding HVR or CDR in a non-human antibody when at least 80%, 85%, 90%, 95% or 100% of corresponding residues (as defined by Kabat) are identical between the respective HVRs or CDRs. The variable region framework sequences of an antibody chain or the constant region of an antibody chain are substantially from a human variable region framework sequence or human constant region respectively when at least 80%, 85%, 90%, 95% or 100% of corresponding residues defined by Kabat are identical.

Although humanized antibodies often incorporate all six HVRs (e.g., CDRs, preferably as defined by Kabat) from a mouse antibody, they can also be made with less than all HVRs or CDRs (e.g., at least 3, 4, or 5) HVRs or CDRs from a mouse antibody (e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; and Tamura et al, Journal of Immunology, 164:1432-1441, 2000).

Certain amino acids from the human variable region framework residues can be selected for substitution based on their possible influence on HVR (e.g., CDR) conformation and/or binding to antigen. Investigation of such possible influences is by modeling, examination of the characteristics of the amino acids at particular locations, or empirical observation of the effects of substitution or mutagenesis of particular amino acids.

For example, when an amino acid differs between a murine variable region framework residue and a selected human variable region framework residue, the human framework amino acid can be substituted by the equivalent framework amino acid from the mouse antibody when it is reasonably expected that the amino acid:
(1) noncovalently binds antigen directly,
(2) is adjacent to an HVR or CDR region,
(3) otherwise interacts with an HVR or CDR region (e.g. is within about 6 Å of such a region);
(4) mediates interaction between the heavy and light chains, or
(5) is the result of somatic mutation in the mouse chain.
(6) is a site of glycosylation.

Framework residues from classes (1)-(3) are sometimes alternately referred to as canonical and vernier residues. Canonical residues refer to framework residues defining the canonical class of the donor CDR loops determining the conformation of a CDR loop (Chothia and Lesk, J. Mol. Biol. 196, 901-917 (1987), Thornton & Martin, J. Mol. Biol., 263, 800-815, 1996). Vernier residues refer to a layer of framework residues that support antigen-binding loop conformations and play a role in fine-tuning the fit of an antibody to antigen (Foote & Winter, 1992, J Mol Bio. 224, 487-499).

Humanized antibodies and methods of making them are reviewed, e.g., in Almagro and Fransson, (2008) Front. Biosci. 13: 1619-1633, and are further described, e.g., in Riechmann et al., (1988) Nature 332:323-329; Queen et al., (1989) Proc. Natl Acad. Sci. USA 86: 10029-10033; U.S. Pat. Nos. 5,821,337, 7,527,791, 6,982,321, and 7,087,409; Kashmiri et al., (2005) Methods 36:25-34 (describing specificity determining region (SDR) grafting); Padlan, (1991) Mol. Immunol. 28:489-498 (describing “resurfacing”); Dall'Acqua et al., (2005) Methods 36:43-60 (describing “FR shuffling”); and Osbourn et al., (2005) Methods 36:61-68 and Klimka et al., (2000) Br. J Cancer, 83:252-260 (describing the “guided selection” approach to FR shuffling).

Human framework regions that may be used for humanization include but are not limited to: framework regions selected using the “best-fit” method (see, e.g., Sims et al. (1993) J. Immunol. 151:2296); framework regions derived from the consensus sequence of human antibodies of a particular subgroup of light or heavy chain variable regions (see, e.g., Carter et al. (1992) Proc. Natl. Acad. Sci. USA, 89:4285; and Presta et al. (1993) J Immunol, 151:2623); human mature (somatically mutated) framework regions or human germline framework regions (see, e.g., Almagro and Fransson, (2008) Front. Biosci. 13:1619-1633); and framework regions derived from screening FR libraries (see, e.g., Baca et al., (1997) J Biol. Chem. 272: 10678-10684 and Rosok et al., (1996) J Biol. Chem. 271:22611-22618).

Nonlimiting exemplary humanized antibodies include humanized antibodies comprising or derived from any of the CDR and/or heavy and/or light chain variable regions as disclosed herein. A specific example of such antibodies includes humanized forms of the mouse antibody 12F3. One such humanized variant of the mouse antibody 12F3 is designated as HGLF, which comprises a mature heavy chain variable region comprising the amino acid sequence of SEQ ID NO: 15 and a mature light chain variable region comprising the amino acid sequence of SEQ ID NO: 30. Humanized antibodies of the invention include variants of the HGLF humanized antibody in which the humanized heavy chain mature variable region shows at least 90%, 95% or 99% identity to SEQ ID NO: 15 and the humanized light chain mature variable region shows at least 90%, 95% or 99% sequence identity to SEQ ID NO: 30. Preferably, in such antibodies some or all of the backmutations in HGLF are retained. In other words, at least 1, 2, 3, 4, 5, 6, or preferably all 7 of heavy chain positions H30, H37, H48, H49, H73, H78, and H93 are occupied by T, V, L, A, N, L, and A, respectively. Likewise, at least 1, 2, 3, 4, or preferably all 4 of light chain positions L2, L38, L49, and L69 are occupied by T, Y, H, and R, respectively. HGLF is described in greater detail in the Examples and which have the sequences shown in FIGS. 5-8.

### D. Exemplary Antibody Constant Regions

For those embodiments in which ABPs are antibodies, the heavy and light chain variable regions of antibodies described herein can be linked to at least a portion of a human constant region. In some embodiments, the human heavy chain constant region is of an isotype selected from IgA, IgG, and IgD. In some embodiments, the human light chain constant region is of an isotype selected from κ and λ. In some embodiments, an antibody described herein comprises a human IgG constant region. In some embodiments, an antibody described herein comprises a human IgG4 heavy chain constant region. In some of these embodiments, an antibody described herein comprises an S228P mutation in the human IgG4 constant region. In some embodiments, an antibody described herein comprises a human IgG4 constant region and a human κ light chain.

Throughout the present specification and claims unless explicitly stated or known to one skilled in the art, the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody.

Human constant regions show allotypic variation and isoallotypic variation between different individuals, that is, the constant regions can differ in different individuals at one or more polymorphic positions. Isoallotypes differ from allotypes in that sera recognizing an isoallotype binds to a non-polymorphic region of a one or more other isotypes. Reference to a human constant region includes a constant region with any natural allotype or any permutation of residues occupying polymorphic positions in natural allotypes. Also, up to 1, 2, 5, or 10 mutations may be present relative to a natural human constant region, such as those indicated above to reduce Fcγ receptor binding or increase binding to FcRn.

In some embodiments, one or several amino acids at the amino or carboxy terminus of the light and/or heavy chain, such as the C-terminal lysine of the heavy chain, may be missing or derivatized in a proportion or all of the molecules.

The choice of constant region depends, in part, whether antibody-dependent cell-mediated cytotoxicity, antibody dependent cellular phagocytosis and/or complement dependent cytotoxicity are desired. For example, human isotopes IgG1 and IgG3 have strong complement-dependent cytotoxicity, human isotype IgG2 weak complement-dependent cytotoxicity and human IgG4 lacks complement-dependent cytotoxicity. Human IgG1 and IgG3 also induce stronger cell-mediated effector functions than human IgG2 and IgG4. Light chain constant regions can be lambda or kappa.

Furthermore, as described in greater detail below, substitutions can be made in the constant regions to reduce or increase effector function such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. USA 103:4005, 2006), or to prolong half-life in humans (see, e.g., Hinton et al., J. Biol. Chem. 279:6213, 2004).

### E. Variants

The antigen binding proteins provided herein also include amino acid sequence variants of the antigen binding proteins provided herein. As an example, variants with improved binding affinity and/or other biological properties of the antibody can be prepared. Amino acid sequence variants of an antigen binding protein can be prepared by introducing appropriate modifications into the nucleotide sequence encoding the antigen binding protein, or by peptide synthesis. Such modifications include, for example, deletions from, and/or insertions into and/or substitutions of residues within the amino acid sequences of the antigen binding protein. Any combination of deletion, insertion, and substitution can be made to arrive at the final construct, provided that the final construct possesses the desired characteristics, e.g., antigen-binding.

### 1. Substitution, Insertion and Deletion Variants

In some embodiments, an antigen binding protein is a variant in that it has one or more amino acid substitutions, deletions and/or insertions relative to an antigen binding protein as described herein. In certain such embodiments, the variant has one or more amino acid substitutions. In further such embodiments, the substitutions are conservative amino acid substitutions.

An amino acid substitution can include but are not limited to the replacement of one amino acid in a polypeptide with another amino acid. Conservative amino acid substitutions can encompass non-naturally occurring amino acid residues, which are typically incorporated by chemical peptide synthesis rather than by synthesis in biological systems. Naturally occurring residues can be divided into classes based on common side chain properties:
(1) hydrophobic: Norleucine, Met, Ala, Val, Leu, Ile;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

Sites of interest for substitutional mutagenesis include the CDRs and FRs. Conservative substitutions are shown in Table 2 below under the heading of “Preferred Substitutions.” More substantial changes are provided in Table 2 under the heading of “Exemplary Substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 2

| Original Residue | Exemplary Substitutions | Preferred Substitutions |
|---|---|---|
| Ala | Val; Leu; Ile | Val |
| Arg | Lys; Gln; Asn | Lys |
| Asn | Gln; His; Asp; Lys; Arg | Gln |
| Asp | Glu; Asn | Glu |
| Cys | Ser; Ala | Ser |
| Gln | Asn; Glu | Asn |
| Glu | Asp; Gln | Asp |
| Gly | Pro; Ala | Ala |
| His | Asn; Gln; Lys; Arg | Arg |
| Ile | Leu; Val; Met; Ala; | Leu |
| | Phe; Norleucine | |
| Leu | Norleucine; Ile; Val; | Ile |
| | Met; Ala; Phe | |
| Lys | Arg; Gln; Asn | Arg |
| Met | Leu; Phe; Ile | Leu |
| Phe | Trp; Leu; Val; | Leu |
| | Ile; Ala; Tyr | |
| Pro | Ala | Ala |
| Ser | Thr; Ala; Cys | Thr |
| Thr | Val; Ser | Ser |
| Trp | Tyr; Phe | Tyr |
| Tyr | Trp; Phe; Thr; Ser | Phe |
| Val | Ile; Leu; Met; Phe; | Leu |
| | Ala; Norleucine | |

Non-conservative substitutions involve exchanging a member of one of these classes for another class.

In altering the amino acid sequence of the antigen binding protein (e.g., anti-ALPP/ALPPL2 antibody), in some embodiments the hydropathic index of amino acids can be considered. Each amino acid has been assigned a hydropathic index on the basis of its hydrophobicity and charge characteristics as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al., 1982, J. Mol. Biol., 157:105-131. It is known that certain amino acids can be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those which are within ±1 are included, and in certain embodiments, those within ±0.5 are included.

It is also understood in the art that the substitution of like amino acids can be made effectively on the basis of hydrophilicity, particularly where the biologically functional protein or peptide (e.g., antibody) thus created is intended for use in immunological embodiments, as in the present case. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0±1); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those which are within ±1 are included, and in certain embodiments, those within ±0.5 are included. One can also identify epitopes from primary amino acid sequences on the basis of hydrophilicity. These regions are also referred to as “epitopic core regions.”

Alterations (e.g., substitutions) can be made in CDRs, e.g., to improve antibody affinity. Such alterations can be made in CDR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain embodiments, substitutions, insertions, or deletions can occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. Such alterations may, for example, be outside of antigen contacting residues in the CDRs. In certain embodiments of the variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

2. Variants with Modified Fc Region

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

In certain embodiments, an antibody variant is prepared that has improved or diminished binding to FcRs are described. (See, e.g., U.S. Pat. No. 6,737,056; WO 2004/056312, and Shields et al., J. Biol. Chem. 9(2): 6591-6604 (2001).) In some embodiments, an antibody variant comprises an Fc region with one or more amino acid substitutions which improve ADCC, e.g., substitutions at positions 298, 333, and/or 334 of the Fc region (EU numbering of residues). For instance, a systemic substitution of solvent-exposed amino acids of human IgG1 Fc region has generated IgG variants with altered FcγR binding affinities (Shields et al., 2001, J. Biol. Chem. 276:6591-604). When compared to parental IgG1, a subset of these variants involving substitutions at Thr256/Ser298, Ser298/Glu333, Ser298/Lys334, or Ser298/Glu333/Lys334 to Ala demonstrate increased in both binding affinity toward FcγR and ADCC activity (Shields et al., 2001, J. Biol. Chem. 276:6591-604; Okazaki et al., 2004, J. Mol. Biol. 336:1239-49).

In some embodiments, alterations are made in the Fc region to alter (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000). For instance, complement fixation activity of antibodies (both C1q binding and CDC activity) can be improved by substitutions at Lys326 and Glu333 (Idusogie et al., 2001, J. Immunol. 166:2571-2575). The same substitutions on a human IgG2 backbone can convert an antibody isotype that binds poorly to C1q and is severely deficient in complement activation activity to one that can both bind C1q and mediate CDC (Idusogie et al., 2001, J. Immunol. 166:2571-75). Several other methods have also been applied to improve complement fixation activity of antibodies. For example, the grafting of an 18-amino acid carboxyl-terminal tail piece of IgM to the carboxyl-termini of IgG greatly enhances their CDC activity. This is observed even with IgG4, which normally has no detectable CDC activity (Smith et al., 1995, J. Immunol. 154:2226-36). Also, substituting Ser444 located close to the carboxy-terminal of IgG1 heavy chain with Cys induced tail-to-tail dimerization of IgG1 with a 200-fold increase of CDC activity over monomeric IgG1 (Shopes et al., 1992, J. Immunol. 148:2918-22). In addition, a bispecific diabody construct with specificity for C1q also confers CDC activity (Kontermann et al., 1997, Nat. Biotech. 15:629-31).

Complement activity can be reduced by mutating at least one of the amino acid residues 318, 320, and 322 of the heavy chain to a residue having a different side chain, such as Ala. Other alkyl-substituted non-ionic residues, such as Gly, Ile, Leu, or Val, or such aromatic non-polar residues as Phe, Tyr, Trp and Pro in place of any one of the three residues also reduce or abolish C1q binding. Ser, Thr, Cys, and Met can be used at residues 320 and 322, but not 318, to reduce or abolish C1q binding activity. Replacement of the 318 (Glu) residue by a polar residue may modify but not abolish C1q binding activity. Replacing residue 297 (Asn) with Ala results in removal of lytic activity but only slightly reduces (about three fold weaker) affinity for C1q. This alteration destroys the glycosylation site and the presence of carbohydrate that is required for complement activation. Any other substitution at this site also destroys the glycosylation site. The following mutations and any combination thereof also reduce C1q binding: D270A, K322A, P329A, and P311S (see WO 06/036291).

The half-life of an antibody as provided herein can be increased or decreased to modify its therapeutic activities. FcRn is a receptor that is structurally similar to MHC Class I antigen that non-covalently associates with 02-microglobulin. FcRn regulates the catabolism of IgGs and their transcytosis across tissues (Ghetie and Ward, 2000, Annu. Rev. Immunol. 18:739-766; Ghetie and Ward, 2002, Immunol. Res. 25:97-113). The IgG-FcRn interaction takes place at pH 6.0 (pH of intracellular vesicles) but not at pH 7.4 (pH of blood); this interaction enables IgGs to be recycled back to the circulation (Ghetie and Ward, 2000, Ann. Rev. Immunol. 18:739-766; Ghetie and Ward, 2002, Immunol. Res. 25:97-113). The region on human IgG₁ involved in FcRn binding has been mapped (Shields et al., 2001, J. Biol. Chem. 276:6591-604). Alanine substitutions at positions Pro238, Thr256, Thr307, Gln311, Asp312, Glu380, Glu382, or Asn434 of human IgG1 enhance FcRn binding (Shields et al., 2001, J. Biol. Chem. 276:6591-604). IgG1 molecules harboring these substitutions have longer serum half-lives. Consequently, these modified IgG₁ molecules may be able to carry out their effector functions, and hence exert their therapeutic efficacies, over a longer period of time compared to unmodified IgG₁. Other exemplary substitutions for increasing binding to FcRn include a Gln at position 250 and/or a Leu at position 428. Other studies have shown that binding of the Fc region to FcRn can be improved by introducing one or more substitutions at one or more the following Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (see, e.g., U.S. Pat. Nos. 7,371,826; and 7,361,740).

3. Antibody Variants with Modified Glycosylation

In certain embodiments, an antibody as provided herein includes one or more modifications so as to increase or decrease the extent to which the antibody is glycosylated. Addition or deletion of glycosylation sites to an antibody can be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure.

Engineering of this glycoform on IgG can significantly improve IgG-mediated ADCC. Addition of bisecting N-acetylglucosamine modifications (Umana et al., 1999, Nat. Biotechnol. 17:176-180; Davies et al., 2001, Biotech. Bioeng. 74:288-94) to this glycoform or removal of fucose (Shields et al., 2002, J. Biol. Chem. 277:26733-40; Shinkawa et al., 2003, J. Biol. Chem. 278:6591-604; Niwa et al., 2004, Cancer Res. 64:2127-33) from this glycoform are two examples of IgG Fc engineering that improves the binding between IgG Fc and FcγR, thereby enhancing Ig-mediated ADCC activity. Antibodies including such substitutions or engineering are included in some of the embodiments provided herein.

In certain embodiments, antibodies are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Patent Application No. US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Other antibodies are further provided which contain bisected oligosaccharides, e.g., in which a biantennary oligosaccharide attached to the Fc region of the antibody is bisected by GlcNAc. Such antibodies may have reduced fucosylation and/or improved ADCC function. Examples of such antibodies are described, e.g., in WO 2003/011878 (Jean-Mairet et al.); U.S. Pat. No. 6,602,684 (Umana et al.); and US 2005/0123546 (Umana et al.). Antibodies with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### 4. Cysteine Engineered Antibody Variants

In some embodiments, an antibody variant as provided herein includes a substitution of the native amino acid to a cysteine residue at amino acid position 234, 235, 237, 239, 267, 298, 299, 326, 330, or 332, preferably an S239C mutation (substitutions of the constant regions are according to the EU index) in a human IgG1 isotype. The presence of an additional cysteine residue allows interchain disulfide bond formation. Such interchain disulfide bond formation can cause steric hindrance, thereby reducing the affinity of the Fc region-FcγR binding interaction. The cysteine residue(s) introduced in or in proximity to the Fc region of an IgG constant region can also serve as sites for conjugation to therapeutic agents (e.g., coupling cytotoxic drugs using thiol specific reagents such as maleimide derivatives of drugs). The presence of a therapeutic agent causes steric hindrance, thereby further reducing the affinity of the Fc region-FcγR binding interaction. Other substitutions at any of positions 234, 235, 236 and/or 237 reduce affinity for Fcγ receptors, particularly FcγRI receptor (see, e.g., U.S. Pat. Nos. 6,624,821, 5,624,821.)

In other cysteine engineered antibody variants, one or more reactive thiol groups are positioned at accessible sites of the antibody and can be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; Al 18 (EU numbering) of the heavy chain; and 5400 (EU numbering) of the heavy chain Fc region. Generating of cysteine engineered antibodies are described, e.g., in U.S. Pat. No. 7,521,541.

### 5. Exemplary Fc Variants

Certain of the ABPs that are provided include the following modifications to the constant region.

### F. Competing Antigen Binding Proteins

The antigen binding proteins provided herein include those that compete with one of the exemplified ABPs or fragments described above for specific binding to ALPP and/or ALPPL2 (e.g., human ALPP of SEQ ID NO: 2 and/or human ALPPL2 of SEQ ID NO: 4). In some of these embodiments, the test and reference ABPs cross-compete with one another. Such ABPs may bind to the same epitope as one of the antigen binding proteins described herein, or to an overlapping epitope. In an embodiment, such ABPs bind to an epitope having the amino acid sequence of SEQ ID NO: 73 and/or SEQ ID NO: 74. ABPs including fragments that compete with the exemplified ABPs are expected to show similar functional properties (e.g., one or more of the activities described above).

In some embodiments, the ABPs that are provided include those that compete with an antibody having: (a) all 6 of the CDRs listed for the same antibody as in SEQ ID NOs: 56-58 or 60-62, and 63-65 or 68-70; (b) a VH and a VL listed for the same antibody as in SEQ ID NOs: 15 and 30.; or (c) a light chain and heavy chain as specified for the same antibody as in SEQ ID NOs: 40 and 50.

G. Antigen Binding Proteins that Bind the Same Epitope

In another embodiment, the antigen binding proteins that are provided include those that bind the same epitope as any of the ABPs described herein. A variety of techniques are available to identify ABPs that bind to the same epitope as one or more of the ABPs described herein. Such methods include, for instance, competition assays such as described herein, screening of peptide fragments, MS-based protein footprinting, alanine or glutamine scanning approaches, and via x-ray analysis of crystals of antigen:antigen binding protein complexes which provides atomic resolution of the epitope.

One approach for determining the epitope or epitope region (an “epitope region” is a region comprising the epitope or overlapping with the epitope) bound by a specific antibody involves assessing binding of an ABP to peptides comprising fragments of ALPP and/or ALPPL2, e.g., non-denatured or denatured fragments. A series of overlapping peptides encompassing the sequence of ALPP and/or ALPPL2 (e.g., human ALPP and/or human ALPPL2) can be prepared and screened for binding, e.g. in a direct ELISA, a competitive ELISA (where the peptide is assessed for its ability to prevent binding of an antibody to ALPP and/or ALPPL2 bound to a well of a microtiter plate), or on a chip. Such peptide screening methods may not be capable of detecting some discontinuous functional epitopes, i.e. functional epitopes that involve amino acid residues that are not contiguous along the primary sequence of the ALPP and/or ALPPL2 polypeptide chain.

In other embodiments, the region(s) containing residues that are in contact with or are buried by an antibody can be identified by mutating specific residues in ALPP and/or ALPPL2 and determining whether the ABP can bind the mutated or variant ALPP and/or ALPPL2 protein. By making a number of individual mutations, residues that play a direct role in binding or that are in sufficiently close proximity to the antibody such that a mutation can affect binding between the antigen binding protein and antigen can be identified. From a knowledge of these amino acids, the domain(s) or region(s) of the antigen that contain residues in contact with the ABP or covered by the antibody can be elucidated. Such a domain can include the binding epitope of an ABP. The general approach for such scanning techniques involves substituting arginine and/or glutamic acid residues (typically individually) for an amino acid in the wild-type polypeptide. These two amino acids are typically used in such scanning techniques because they are charged and bulky and thus have the potential to disrupt binding between an ABP and the ALPP and/or ALPPL2 in the region of the ALPP and/or ALPPL2 where the mutation is introduced. Arginines that exist in the wild-type antigen are replaced with glutamic acid. A variety of such individual mutants are obtained and the collected binding results analyzed to determine what residues affect binding (see, e.g., Nanevicz, T., et al., 1995, J. Biol. Chem., 270:37, 21619-21625 and Zupnick, A., et al., 2006, J. Biol. Chem., 281:29, 20464-20473).

An alternative approach for identifying an epitope is by MS-based protein footprinting, such as hydrogen/deuterium exchange mass spectrometry (HDX-MS) and Fast Photochemical Oxidation of Proteins (FPOP). Methods for conducting HDX-MS are described, for example, in Wei et al. (2014) Drug Discovery Today 19:95. Methods for performing FPOP are described, for instance, in Hambley and Gross (2005) J. American Soc. Mass Spectrometry 16:2057.

The epitope bound by an ABP can also be determined by structural methods, such as an X-ray crystal structure determination, molecular modeling, and nuclear magnetic resonance (NMR) spectroscopy, including NMR determination of the H-D exchange rates of labile amide hydrogens in the antigen when free and when bound in a complex with an ABP (see, e.g., Zinn-Justin et al. (1992) Biochemistry 31, 11335-11347; and Zinn-Justin et al. (1993) Biochemistry 32, 6884-6891).

X-ray crystallography analyses can be accomplished using any of the known methods in the art. Examples of crystallization methods are described, for instance, by Giege et al. (1994) Acta Crystallogr. D50:339-350; and McPherson (1990) Eur. J. Biochem. 189:1-23). Such crystallization approaches include microbatch (e.g. Chayen (1997) Structure 5:1269-1274), hanging-drop vapor diffusion (e.g. McPherson (1976) J. Biol. Chem. 251:6300-6303), seeding and dialysis. Once formed, the ABP:antigen crystals themselves can be studied using well-known X-ray diffraction techniques and can be refined using computer software such as X-PLOR (Yale University, 1992, distributed by Molecular Simulations, Inc.; see e.g. Blundell & Johnson (1985) Meth. Enzymol. 114 & 115, H. W. Wyckoff et al., eds., Academic Press; U.S. Patent Application Publication No. 2004/0014194), and BUSTER (Bricogne (1993) Acta Cryst. D49:37-60; Bricogne (1997) Meth. Enzymol. 276A:361-423, Carter & Sweet, eds.; Roversi et al. (2000) Acta Cryst. D56:1313-1323).

In some embodiments, the ABP binds a continuous epitope. In a preferred embodiment, the ABP binds an epitope having an amino acid sequence of SEQ ID NO: 73 and/or SEQ ID NO: 74.

### H. Other Exemplary Formats

An antigen binding protein (e.g., an antibody or antigen-binding fragment thereof) can be a single polypeptide, or can include two, three, four, five, six, seven, eight, nine, or ten (the same or different) polypeptides. In some embodiments where the antibody or antigen-binding fragment thereof is a single polypeptide, the antibody or antigen-binding fragment can include a single antigen-binding domain or two antigen-binding domains. In some embodiments where the antibody or antigen-binding fragment is a single polypeptide and includes two antigen-binding domains, the first and second antigen-binding domains can be identical or different from each other (and can specifically bind to the same or different antigens or epitopes).

The different parts of the antigen binding proteins described herein can be arranged in various configurations to obtain additional antigen binding proteins. For example, in some embodiments where the antibody or the antigen-binding fragment is a single polypeptide, the first antigen-binding domain and the second antigen-binding domain (if present) can each be independently selected from the group of: a VH domain, a VHH domain, a VNAR domain, and a scFv. In some embodiments where the antibody or the antigen-binding fragment is a single polypeptide, the antibody or antigen-binding fragment can be a BiTE®, a (scFv)₂, a nanobody, a nanobody-HSA, a DART, a TandAb, a scDiabody, a scDiabody-CH3, scFv-CH-CL-scFv, a HSAbody, scDiabody-HAS, a tandem-scFv, an Adnectin, a DARPin, a fibronectin, and a DEP conjugate. Additional examples of antigen-binding domains that can be used when the antibody or antigen-binding fragment is a single polypeptide are known in the art.

A V_{H}H domain is a single monomeric variable antibody domain that can be found in camelids. A V_{NAR} domain is a single monomeric variable antibody domain that can be found in cartilaginous fish. Non-limiting aspects of V_{H}H domains and V_{NAR} domains are described in, e.g., Cromie et al., Curr. Top. Med. Chem. 15:2543-2557, 2016; De Genst et al., Dev. Comp. Immunol. 30:187-198, 2006; De Meyer et al., Trends Biotechnol. 32:263-270, 2014; Kijanka et al., Nanomedicine 10:161-174, 2015; Kovaleva et al., Expert. Opin. Biol. Ther. 14:1527-1539, 2014; Krah et al., Immunopharmacol. Immunotoxicol. 38:21-28, 2016; Mujic-Delic et al., Trends Pharmacol. Sci. 35:247-255, 2014; Muyldermans, J. Biotechnol. 74:277-302, 2001; Muyldermans et al., Trends Biochem. Sci. 26:230-235, 2001; Muyldermans, Ann. Rev. Biochem. 82:775-797, 2013; Rahbarizadeh et al., Immunol. Invest. 40:299-338, 2011; Van Audenhove et al., EBioMedicine 8:40-48, 2016; Van Bockstaele et al., Curr Opin. Investig. Drugs 10:1212-1224, 2009; Vincke et al., Methods Mol. Biol. 911:15-26, 2012; and Wesolowski et al., Med. Microbiol. Immunol. 198:157-174, 2009.

In some embodiments where the antibody or antigen-binding fragment is a single polypeptide and includes two antigen-binding domains, the first antigen-binding domain and the second antigen-binding domain can both be VHH domains, or at least one antigen-binding domain can be a VHH domain. In some embodiments where the antibody or antigen-binding fragment is a single polypeptide and includes two antigen-binding domains, the first antigen-binding domain and the second antigen-binding domain are both V_{NAR} domains, or at least one antigen-binding domain is a V_{NAR} domain. In some embodiments where the antibody or antigen-binding domain is a single polypeptide, the first antigen-binding domain is a scFv domain. In some embodiments where the antibody or antigen-binding fragment is a single polypeptide and includes two antigen-binding domains, the first antigen-binding domain and the second antigen-binding domain can both be scFv domains, or at least one antigen-binding domain can be a scFv domain.

In some embodiments, the antibody or antigen-binding fragment can include two or more polypeptides (e.g., two, three, four, five, six, seven, eight, nine, or ten polypeptides). In some embodiments where the antibody or antigen-binding fragment includes two or more polypeptides, two, three, four, five or six of the polypeptides of the two or more polypeptides can be identical.

In some embodiments where the antibody or antigen-binding fragment includes two or more polypeptides (e.g., two, three, four, five, six, seven, eight, nine, or ten polypeptides), two or more of the polypeptides of the antibody or antigen-binding fragment can assemble (e.g., non-covalently assemble) to form one or more antigen-binding domains, e.g., an antigen-binding fragment of an antibody (e.g., any of the antigen-binding fragments of an antibody described herein), a VHH-scAb, a VHH-Fab, a Dual scFab, a F(ab′)₂, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a κλ-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)₂-scFv₂, a scFv-KIH, a Fab-scFv-Fc, a tetravalent HCAb, a scDiabody-Fc, a Diabody-Fc, a tandem scFv-Fc, a VHH-Fc, a tandem VHH-Fc, a VHH-Fc KiH, a Fab-VHH-Fc, an Intrabody, a dock and lock, an ImmTAC, an IgG-IgG conjugate, a Cov-X-Body, a scFv1-PEG-scFv2, an Adnectin, a DARPin, a fibronectin, and a DEP conjugate. See, e.g., Spiess et al., Mol. Immunol. 67:95-106, 2015, incorporated in its entirety herewith, for a description of these elements.

In some embodiments, the antigen binding protein is based upon a non-immunoglobulin scaffold. Examples of other scaffolds into which the binding domains (e.g., HVRs or CDRs) such as described herein can be inserted or grafted include, but are not limited to, human fibronectin (e.g., the 10^{th} extracellular domain of human fibronectin III), neocarzinostatin CBM4-2, anticalines derived from lipocalins, designed ankyrin repeat domains (DARPins), protein-A domain (protein Z), Kunitz domains, Im9, TPR proteins, zinc finger domains, pVIII, GC4, transferrin, B-domain of SPA, Sac7d, A-domain, SH3 domain of Fyn kinase, and C-type lectin-like domains (see, e.g., Gebauer and Skerra (2009) Curr. Opin. Chem. Biol., 13:245-255; Binz et al. (2005) Nat. Biotech. 23:1257-1268; and Yu et al. (2017) Annu Rev Anal Chem 10:293-320, each of which is incorporated herein by reference in its entirety).

### IV. Antigen Binding Protein Expression and Production

### A. Nucleic Acid Molecules Encoding Antigen Binding Proteins

Nucleic acid molecules that encode for the antigen binding proteins described herein, or portions thereof, are also provided. Such nucleic acids include, for example: 1) those encoding an antigen binding protein (e.g., an antibody or a fragment thereof), or a derivative, or variant thereof; 2) polynucleotides encoding a heavy and/or light chain, VH and/or VL domains, or 1 or more of the HVRs or CDRs located within a variable domain (e.g., 1, 2 or all 3 of the VH HVRs or CDRs or 1, 2 or all 3 of the VL HVRs or CDRs); 3) polynucleotides sufficient for use as hybridization probes, PCR primers or sequencing primers for identifying, analyzing, mutating or amplifying such encoding polynucleotides; 4) anti-sense nucleic acids for inhibiting expression of such encoding polynucleotides, and 5) complementary sequences of the foregoing. The nucleic acids can be any length. They can be, for example, 5, 10, 15, 20, 25, 30, 35, 40, 45, 50, 75, 100, 125, 150, 175, 200, 250, 300, 350, 400, 450, 500, 750, or 1,000 or more nucleotides in length, and/or can comprise one or more additional sequences, for example, regulatory sequences, and/or be part of a larger nucleic acid, for example, a vector. The nucleic acids can be single-stranded or double-stranded.

The nucleic acid molecules can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., other chromosomal DNA, e.g., the chromosomal DNA that is linked to the isolated DNA in nature) or proteins, by standard techniques, including alkaline/SDS treatment, CsCl banding, column chromatography, restriction enzymes, agarose gel electrophoresis and others well known in the art. See, F. Ausubel, et al., ed. (1987) Current Protocols in Molecular Biology, Greene Publishing and Wiley Interscience, New York. A nucleic acid described herein can be, for example, DNA or RNA and may or may not contain intronic sequences. In certain embodiments, the nucleic acid is a cDNA molecule.

In an embodiment, the nucleic acid molecule that encodes a VH sequence of an antibody provided herein comprises SEQ ID NO: 71. In another embodiment, the nucleic acid molecule that encodes a VL sequence of an antibody provided herein comprises SEQ ID NO: 72. In a further embodiment, the nucleic acid molecules that encode the VH and VL sequences of antibodies provided herein comprise SEQ ID NO: 71 and SEQ ID NO: 72, respectively.

Thus, nucleic acid molecules comprising polynucleotides that encode one or more chains of an ABP, such as anti-ALPP/ALPPL2 antibodies, are provided. In some embodiments, a nucleic acid molecule comprises a polynucleotide that encodes a heavy chain or a light chain of an ABP (e.g., an anti-ALPP/ALPPL2 antibody). In some embodiments, a nucleic acid molecule comprises both a polynucleotide sequence that encodes a heavy chain and a polynucleotide sequence that encodes a light chain, of an ABP (e.g., an anti-ALPP/ALPPL2 antibody). In some embodiments, a first nucleic acid molecule comprises a first polynucleotide sequence that encodes a heavy chain and a second nucleic acid molecule comprises a second polynucleotide sequence that encodes a light chain.

In one embodiment, the nucleic acid molecule comprises a polynucleotide encoding the VH of one of the antibodies provided herein. In another embodiment, the nucleic acid comprises a polynucleotide encoding the VL of one of the antibodies provided herein. In still another embodiment, the nucleic acid encodes both the VH and the VL of one of the antibodies provided herein. In certain embodiments, the nucleic acid molecule comprises a polynucleotide encoding the amino acid sequence of SEQ ID NO: 15 or SEQ ID NO: 30.

In a particular embodiment, the nucleic acid encodes a variant of one or more of the above amino acid sequences (e.g., the heavy chain and/or light chain amino acid sequences, or the VH and/or VL amino acid sequences disclosed herein), wherein the variants has at most 25 amino acid modifications, such as at most 20, such as at most 15, 14, 13, 12 or 11 amino acid modifications, such as 10, 9, 8, 7, 6, 5, 4, 3, 2 or 1 amino-acid modifications, such as deletions or insertions, preferably substitutions, such as conservative substitutions.

Also provided are nucleic acid molecules that have at least 80%, 85%, 90% (e.g., 95%, 96%, 97%, 98%, or 99%) sequence identity to any of the aforementioned sequences. Thus, for example, in certain embodiments, the nucleic comprises a nucleotide sequence that encodes the heavy and/or light chain sequence or the VH and/or VL sequence of one of the antigen binding proteins disclosed herein.

Once nucleic acids encoding VH and VL segments are obtained, these nucleic acids can be further manipulated by standard recombinant DNA techniques, for example to convert the variable region genes to full-length antibody chain genes, to Fab fragment genes or to a scFv gene. In these manipulations, a VL- or VH-encoding nucleic acid is operatively linked to another nucleic acid encoding another polypeptide, such as an antibody constant region or a flexible linker.

The isolated nucleic acid encoding the VH region can be converted to a full-length heavy chain gene by operatively linking the VH-encoding nucleic acid to another nucleic acid molecule encoding heavy chain constant regions (hinge, CH1, CH2 and/or CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and nucleic acid fragments encompassing these regions can be obtained by standard PCR amplification. The heavy chain constant region can be an IgG1, IgG2, IgG3, IgG4, IgA, IgE, IgM or IgD constant region, for example, an IgG1 region. For a Fab fragment heavy chain gene, the VH-encoding nucleic can be operatively linked to another nucleic acid molecule encoding only the heavy chain CH1 constant region.

The isolated nucleic acid molecule encoding the VL region can be converted to a full-length light chain gene (as well as a Fab light chain gene) by operatively linking the VL-encoding nucleic acid molecule to another nucleic acid molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and nucleic acid fragments encompassing these regions can be obtained by standard PCR amplification. The light chain constant region can be a kappa or lambda constant region.

To create a scFv gene, the VH- and VL-encoding nucleic acid fragments are operatively linked to another fragment encoding a flexible linker, e.g., encoding the amino acid sequence (Gly₄-Ser)₃, such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., (1990) Nature 348:552-554).

In another aspect, nucleic acid molecules that are suitable for use as primers or hybridization probes for the detection of nucleic acid sequences are also provided. A nucleic acid molecule can comprise only a portion of a nucleic acid sequence encoding a full-length polypeptide, for example, a fragment that can be used as a probe or primer or a fragment encoding an active portion (e.g., ALPP and/or ALPPL2 binding portion) of a polypeptide.

Probes based on the sequence of a nucleic acid can be used to detect the nucleic acid or similar nucleic acids, for example, transcripts encoding a polypeptide. The probe can comprise a label group, e.g., a radioisotope, a fluorescent compound, an enzyme, or an enzyme co-factor. Such probes can be used to identify a cell that expresses the polypeptide.

Vectors, including expression vectors, comprising one or more nucleic acids encoding one or more components of the ABPs (e.g. VH and/or VL; and light chains, and/or heavy chains) are also provided. An expression vector can include, but is not limited to, sequences that affect or control transcription, translation, and, if introns are present, affect RNA splicing of a coding region operably linked thereto. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals.

The expression vector can also include a secretory signal peptide sequence that is operably linked to the coding sequence of interest, such that the expressed polypeptide can be secreted by the recombinant host cell, for more facile isolation of the polypeptide of interest from the cell, if desired.

Expression and cloning vectors of the invention will typically contain a promoter that is recognized by the host organism and operably linked to the molecule encoding the polypeptide. A large number of promoters, recognized by a variety of potential host cells, are well known. A suitable promoter is operably linked to the DNA encoding e.g., heavy chain, light chain, or other component of the antibodies and antigen-binding fragments of the invention, by removing the promoter from the source DNA by restriction enzyme digestion and inserting the desired promoter sequence into the vector. Suitable promoters for use with yeast hosts are also well known in the art. Yeast enhancers are advantageously used with yeast promoters. Suitable promoters for use with mammalian host cells are well known and include, but are not limited to, those obtained from the genomes of viruses such as polyoma virus, fowlpox virus, adenovirus (such as Adenovirus serotypes 2, 8, or 9), bovine papilloma virus, avian sarcoma virus, cytomegalovirus, retroviruses, hepatitis-B virus and Simian Virus 40 (SV40). Other suitable mammalian promoters include heterologous mammalian promoters, for example, heat-shock promoters and the actin promoter.

Additional specific promoters that can be utilized include, but are not limited to: SV40 early promoter (Benoist and Chambon, 1981, Nature 290:304-310); CMV promoter (Thornsen et al., 1984, Proc. Natl. Acad. U.S.A. 81:659-663); the promoter contained in the 3′ long terminal repeat of Rous sarcoma virus (Yamamoto et al., 1980, Cell 22:787-797); herpes thymidine kinase promoter (Wagner et al., 1981, Proc. Natl. Acad. Sci. U.S.A. 78: 1444-1445); promoter and regulatory sequences from the metallothionine gene (Prinster et al., 1982, Nature 296:39-42); and prokaryotic promoters such as the beta-lactamase promoter (Villa-Kamaroff et al., 1978, Proc. Natl. Acad. Sci. U.S.A. 75:3727-3731); or the tac promoter (DeBoer et al., 1983, Proc. Natl. Acad. Sci. U.S.A. 80:21-25).

In certain embodiments, nucleic acids encoding the different components of the ABP can be inserted into the same expression vector. For instance, the nucleic acid encoding an anti-ALPP/ALPPL2 antibody light chain or variable region can be cloned into the same vector as the nucleic acid encoding an anti-ALPP/ALPPL2 antibody heavy chain or variable region. In such embodiments, the two nucleic acids may be separated by an internal ribosome entry site (IRES) and under the control of a single promoter such that the light chain and heavy chain are expressed from the same mRNA transcript. Alternatively, the two nucleic acids can be under the control of two separate promoters such that the light chain and heavy chain are expressed from two separate mRNA transcripts. In some embodiments, the nucleic acid encoding the anti-ALPP/ALPPL2 antibody light chain or variable region is cloned into one expression vector and the nucleic acid encoding the anti-ALPP/ALPPL2 antibody heavy chain or variable region is cloned into a second expression vector. In such embodiments, a host cell may be co-transfected with both expression vectors to produce complete antibodies or antigen-binding fragments of the invention.

### B. Host Cells

Ater the vector has been constructed and the one or more nucleic acid molecules encoding the components of the ABPs described herein has been inserted into the proper site(s) of the vector or vectors, the completed vector(s) may be inserted into a suitable host cell for amplification and/or polypeptide expression.

Thus, in another aspect, host cells comprising nucleic acid molecules or vectors such as described herein are also provided. In various embodiments, ABP heavy chains and/or antilight chains can be expressed in prokaryotic cells, such as bacterial cells, or in eukaryotic cells, such as fungal cells (such as yeast), plant cells, insect cells, and mammalian cells. The selection of an appropriate host cell depends upon various factors, such as desired expression levels, polypeptide modifications that are desirable or necessary for activity (such as glycosylation or phosphorylation) and ease of folding into a biologically active molecule.

Introduction of one or more nucleic acids into a desired host cell can be accomplished by any method, including but not limited to, calcium phosphate transfection, DEAE-dextran mediated transfection, cationic lipid-mediated transfection, electroporation, transduction, infection, etc. Nonlimiting exemplary methods are described, e.g., in Sambrook et al., Molecular Cloning, A Laboratory Manual, 3rd ed. Cold Spring Harbor Laboratory Press (2001). Nucleic acids may be transiently or stably transfected in the desired host cells, according to any suitable method.

Exemplary prokaryotic host cells include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacillus, such as B. subtilis and B. licheniformis, Pseudomonas, and Streptomyces.

Yeast can also be used as host cells including, but not limited to, S. cerevisae, S. pombe; or K. lactis.

A variety of mammalian cell lines can be used as hosts and include, but are not limited to, immortalized cell lines available from the American Type Culture Collection (ATCC), including but not limited to Chinese hamster ovary (CHO) cells, including CHOK1 cells (ATCC CCL61), DXB-11, DG-44, and Chinese hamster ovary cells/-DHFR (CHO, Urlaub et al., Proc. Natl. Acad. Sci. USA 77: 4216, 1980); monkey kidney CV1 line transformed by SV40 (COS-7, ATCC CRL 1651); human embryonic kidney line (293 or 293 cells subcloned for growth in suspension culture, (Graham et al., J. Gen Virol. 36: 59, 1977); baby hamster kidney cells (BHK, ATCC CCL 10); mouse sertoli cells (TM4, Mather, Biol. Reprod. 23: 243-251, 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TM cells (Mather et al., Annals N.Y Acad. Sci. 383: 44-68, 1982); MRC 5 cells or FS4 cells; mammalian myeloma cells, and a number of other cell lines.

Once a suitable host cell has been prepared, it can be used to express the desired ABP. Thus, in a further aspect, methods for producing an ABP as described herein are also provided. In general, such methods comprise culturing a host cell comprising one or more expression vectors as described herein in a culture medium under conditions permitting expression of the ABP as encoded by the one or more expression vectors; and recovering the ABP from the culture medium.

In some embodiments, the ABP is produced in a cell-free system. Nonlimiting exemplary cell-free systems are described, e.g., in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

### V. Antigen Binding Protein Conjugates

The ABP that are provided herein can be conjugated to cytotoxic or cytostatic moieties (including pharmaceutically compatible salts thereof) to form a conjugate, such as an antibody drug conjugate (ADC). Particularly suitable moieties for conjugation to ABPs (e.g., antibodies) are cytotoxic agents (e.g., chemotherapeutic agents), prodrug converting enzymes, radioactive isotopes or compounds, or toxins (these moieties being collectively referred to as a therapeutic agent). For example, an ABP (e.g., an anti-ALPP/ALPPL2 antibody) can be conjugated to a cytotoxic agent such as a chemotherapeutic agent, or a toxin (e.g., a cytostatic or cytocidal agent such as, for example, abrin, ricin A, pseudomonas exotoxin, or diphtheria toxin). Examples of useful classes of cytotoxic agents include, for example, DNA minor groove binders, DNA alkylating agents, and tubulin inhibitors. Exemplary cytotoxic agents include, for example, auristatins, camptothecins, calicheamicins, duocarmycins, etoposides, maytansinoids (e.g., DM1, DM2, DM3, DM4), taxanes, benzodiazepines (e.g., pyrrolo[1,4]benzodiazepines, indolinobenzodiazepines, and oxazolidinobenzodiazepines) and vinca alkaloids.

In one embodiment, an ABP (e.g., an anti-ALPP/ALPPL2 antibody) is conjugated to a pro-drug converting enzyme. The pro-drug converting enzyme can be recombinantly fused to the antibody or chemically conjugated thereto using known methods. Exemplary pro-drug converting enzymes are carboxypeptidase G2, betaglucuronidase, penicillin-V-amidase, penicillin-G-amidase, β-lactamase, β-glucosidase, nitroreductase and carboxypeptidase A.

Techniques for conjugating therapeutic agents to proteins, and in particular to antibodies, are well-known. (See, e.g., Alley et al., Current Opinion in Chemical Biology 2010 14:1-9; Senter, Cancer J., 2008, 14(3):154-169.) The therapeutic agent can be conjugated in a manner that reduces its activity unless it is cleaved off the antibody (e.g., by hydrolysis, by proteolytic degradation, or by a cleaving agent). In some aspects, the therapeutic agent is attached to the antibody with a cleavable linker that is sensitive to cleavage in the intracellular environment of the ALPP-expressing cancer cell but is not substantially sensitive to the extracellular environment, such that the conjugate is cleaved from the antibody when it is internalized by the ALPP-expressing cancer cell (e.g., in the endosomal or, for example by virtue of pH sensitivity or protease sensitivity, in the lysosomal environment or in the caveolear environment). In some aspects, the therapeutic agent can also be attached to the antibody with a non-cleavable linker.

Typically, the ADC comprises a linker region between the therapeutic agent and the anti-ABP (e.g., anti-ALPP/ALPPL2 antibody). The linker generally is cleavable under intracellular conditions, such that cleavage of the linker releases the therapeutic agent from the antibody in the intracellular environment (e.g., within a lysosome or endosome or caveolea). The linker can be, e.g., a peptidyl linker that is cleaved by an intracellular peptidase or protease enzyme, including a lysosomal or endosomal protease. Cleaving agents can include cathepsins B and D and plasmin (see, e.g., Dubowchik and Walker, Pharm. Therapeutics 83:67-123, 1999). Most typical are peptidyl linkers that are cleavable by enzymes that are present in ALPP-expressing cells. For example, a peptidyl linker that is cleavable by the thiol-dependent protease cathepsin-B, which is highly expressed in cancerous tissue, can be used (e.g., a linker comprising a Phe-Leu or a Val-Cit peptide).

The cleavable linker can be pH-sensitive, i.e., sensitive to hydrolysis at certain pH values. Typically, the pH-sensitive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; 5,622,929; Dubowchik and Walker, Pharm. Therapeutics 83:67-123, 1999; Neville et al., Biol. Chem. 264:14653-14661, 1989.) Such linkers are relatively stable under neutral pH conditions, such as those in the blood, but are unstable at below pH 5.5 or 5.0, the approximate pH of the lysosome.

Other linkers are cleavable under reducing conditions (e.g., a disulfide linker). Disulfide linkers include those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT. (See, e.g., Thorpe et al., Cancer Res. 47:5924-5931, 1987; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935.)

The linker can also be a malonate linker (Johnson et al., Anticancer Res. 15:1387-93, 1995), a maleimidobenzoyl linker (Lau et al., Bioorg-Med-Chem. 3:1299-1304, 1995), or a 3′-N-amide analog (Lau et al., Bioorg-Med-Chem. 3:1305-12, 1995).

In other embodiments, the linker is a non-cleavable linker, such as an maleimido-alkylene- or maleimide-aryl linker that is directly attached to the therapeutic agent and released by proteolytic degradation of the antibody.

Typically, the linker is not substantially sensitive to the extracellular environment, meaning that no more than about 20%, typically no more than about 15%, more typically no more than about 10%, and even more typically no more than about 5%, no more than about 3%, or no more than about 1% of the linkers in a sample of the ADC is cleaved when the ADC is present in an extracellular environment (e.g., in plasma). Whether a linker is not substantially sensitive to the extracellular environment can be determined, for example, by incubating independently with plasma both (a) the ADC (the “ADC sample”) and (b) an equal molar amount of unconjugated antibody or therapeutic agent (the “control sample”) for a predetermined time period (e.g., 2, 4, 8, 16, or 24 hours) and then comparing the amount of unconjugated antibody or therapeutic agent present in the ADC sample with that present in control sample, as measured, for example, by high performance liquid chromatography.

The linker can also promote cellular internalization. The linker can promote cellular internalization when conjugated to the therapeutic agent (i.e., in the milieu of the linker-therapeutic agent moiety of the ADC or ADC derivate as described herein). Alternatively, the linker can promote cellular internalization when conjugated to both the therapeutic agent and the antigen binding protein (e.g., anti-ALPP/ALPPL2 antibody) (i.e., in the milieu of the ADC as described herein).

Exemplary antibody-drug conjugates include auristatin based antibody-drug conjugates meaning that the drug component is an auristatin drug. Auristatins bind tubulin, have been shown to interfere with microtubule dynamics and nuclear and cellular division, and have anticancer activity. Typically the auristatin based antibody-drug conjugate comprises a linker between the auristatin drug and the ABP (e.g., anti-ALPP/ALPPL2 antibody). The linker can be, for example, a cleavable linker (e.g., a peptidyl linker) or a non-cleavable linker (e.g., linker released by degradation of the antibody). The auristatin can be auristatin E or a derivative thereof. The auristatin can be, for example, an ester formed between auristatin E and a keto acid. For example, auristatin E can be reacted with paraacetyl benzoic acid or benzoylvaleric acid to produce AEB and AEVB, respectively. Other typical auristatins include MMAF, and MMAE. The synthesis and structure of exemplary auristatins are described in U.S. Pat. Nos. 7,659,241, 7,498,298, 2009-0111756, 2009-0018086, and 7,968,687 each of which is incorporated herein by reference in its entirety and for all purposes.

Exemplary auristatin based antibody drug conjugates include mc-vc-PABC-MMAE (also referred to herein as vcMMAE or 1006), me-vc-PABC-MMAF, me-MMAF, and mp-dLAE-PABC-MMAE (also referred to herein as dLAE-MMAE or mp-dLAE-MMAE or 7092), antibody drug conjugates as shown below wherein Ab is an ABP (e.g., an anti-ALPP/ALPPL2 antibody as described herein) and val-cit (vc) represents the valine-citrulline dipeptide, and dLAE represents the D-leucine-alanine-glutamic acid tripeptide:
tripeptide:

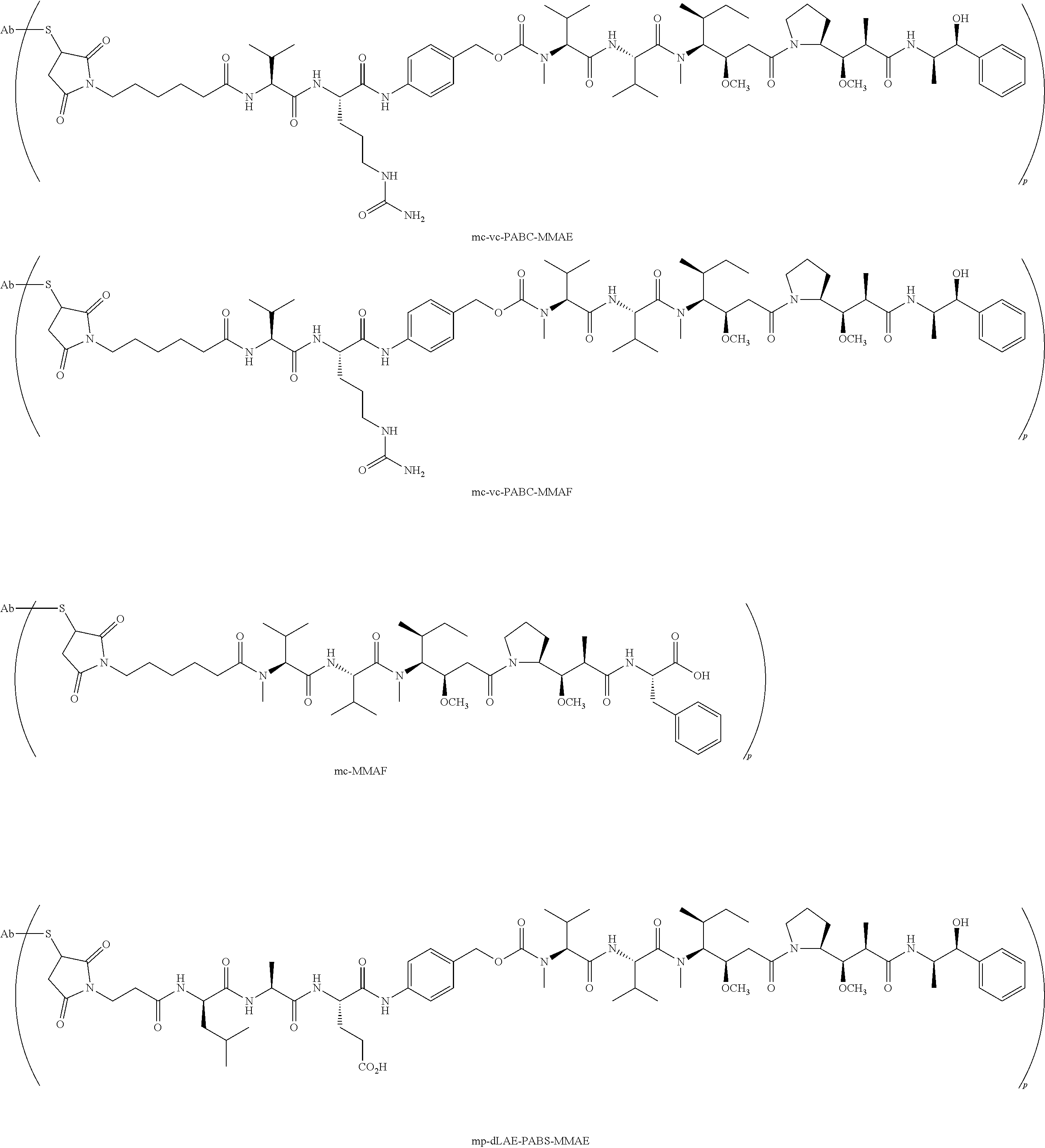

or a pharmaceutically acceptable salt thereof. The drug loading is represented by p, the number of drug-linker moieties per antibody. Depending on the context, p can represent the average number of drug-linker moieties per antibody in a composition of antibodies, also referred to the average drug loading. P ranges from 1 to 20 and is preferably from 1 to 12 or 1 to 8. In some preferred embodiments, when p represents the average drug loading, p ranges from about 2 to about 5. In some embodiments, p is about 2, about 3, about 4, or about 5. The average number of drugs per antibody in a preparation may be characterized by conventional means such as mass spectroscopy, HIC, ELISA assay, and HPLC. In some aspects, the ABP (e.g., anti-ALPP/ALPPL2 antibody) is attached to the drug-linker through a cysteine residue of the antibody. In some embodiments, the cysteine residue is one that is engineered into the antibody. In other aspects, the cysteine residue is an interchain disulfide cysteine residue.

### VI. Therapeutic Applications

### A. Methods of Treating Diseases

In another aspect, methods of treating disorders associated with cells that express ALPP and/or ALPPL2, e.g., cancers, are provided. The cells may or may not express elevated levels of ALPP and/or ALPPL2 relative to cells that are not associated with a disorder of interest. Thus, certain embodiments involve the use of the ABPs described herein (e.g, anti-ALPP/ALPPL2 antibodies), either as a naked antibody or as a conjugate (e.g., an antibody drug conjugate) to treat a subject, for example a subject with a cancer. In some of these embodiments, the method comprises administering an effective amount of an ABP (e.g, an anti-ALPP/ALPPL2 antibody) or a conjugate (e.g., an anti-ALPP/ALPPL2 ADC), or a composition comprising such an ABP or conjugate to a subject in need thereof. In certain exemplary embodiments, the method comprises treating cancer in a cell, tissue, organ, animal or patient. Most typically, the treatment method comprises treating a cancer in a human. In some embodiments the treatment involves monotherapy. In other methods, the antigen binding protein is administered as part of a combination therapy with one or more other therapeutic agents, surgery and/or radiation.

Positive therapeutic effects in cancer can be measured in a number of ways (See, e.g., W. A. Weber, J. Null. Med. 50:15-10S (2009); and Eisenhauer et al., Eur. J Cancer 45:228-247 (2009)). In some embodiments, response to treatment with an ABP or conjugate is assessed using RECIST 1.1 criteria. In some embodiments, the treatment achieved by a therapeutically effective amount is any of inhibition of further tumor growth, inducement of tumor regression, a partial response (PR), a complete response (CR), progression free survival (PFS), disease free survival (DFS), objective response (OR) or overall survival (OS). In some embodiments, treatment delays or prevents the onset of metastasis. Progress in treatment can be monitored using various methods. For instance, inhibition can result in reduced tumor size and/or a decrease in metabolic activity within the tumor. Both of these parameters can be measured by MRI or PET scans, for example. Inhibition can also be monitored by biopsy to ascertain the level of necrosis, tumor cell death and the level of vascularity within the tumor. The dosage regimen of a therapy described herein that is effective to treat a cancer patient may vary according to factors such as the disease state, age, and weight of the patient, and the ability of the therapy to elicit an anti-cancer response in the subject. While an embodiment of the treatment method, medicaments and uses of the present invention may not be effective in achieving a positive therapeutic effect in every subject, it should do so in a statistically significant number of subjects as determined by any statistical test known in the art such as the Student's t-test, the chi2-test, the U-test according to Mann and Whitney, the Kruskal-Wallis test (H-test), Jonckheere-Terpstra-test and the Wilcoxon-test.

“RECIST 1.1 Response Criteria” as used herein means the definitions set forth in Eisenhauer et al., Eur. J Cancer 45:228-247 (2009) for target lesions or non-target lesions, as appropriate, based on the context in which response is being measured.

The effective amount of the ABP (e.g., anti-ALPP/ALPPL2 antibody) or ADC can be administered in one or more administrations, applications or dosages and is not intended to be limited to a particular formulation or administration route. Generally, a therapeutically effective amount of active component is in the range of 0.1 mg/kg to 100 mg/kg, e.g., 1 mg/kg to 100 mg/kg, 1 mg/kg to 10 mg/kg.

Exemplary dosages for the ABP (e.g, anti-ALPP/ALPPL2 antibody) are, for example, 0.1 mg/kg to 50 mg/kg of the patient's body weight, more typically 1 mg/kg to 30 mg/kg, 1 mg/kg to 20 mg/kg, 1 mg/kg to 15 mg/kg, 1 mg/kg to 12 mg/kg, or 1 mg/kg to 10 mg/kg1, or 2 mg/kg to 30 mg/kg, 2 mg/kg to 20 mg/kg, 2 mg/kg to 15 mg/kg, 2 mg/kg to 12 mg/kg, or 2 mg/kg to 10 mg/kg, or 3 mg/kg to 30 mg/kg, 3 mg/kg to 20 mg/kg, 3 mg/kg to 15 mg/kg, 3 mg/kg to 12 mg/kg, or 3 mg/kg to 10 mg/kg.

Exemplary dosages for the ABP (e.g, anti-ALPP/ALPPL2 antibody) are, for example are, for example, 0.01 mg/kg to 10 mg/kg, 0.1 mg/kg to 10 mg/kg, 0.3 mg/kg to 3 mg/kg, 0.5 mg/kg to 3 mg/kg, 1 mg/kg to 7.5 mg/kg, or 2 mg/kg to 7.5 mg/kg or 3 mg/kg to 7.5 mg/kg of the subject's body weight, or 0.1-20, or 0.5-5 mg/kg body weight (e.g., 0.5, 1, 2, 3, 4, 5, 6, 7, 8, 9 or 10 mg/kg) or 10-1500 or 200-1500 mg as a fixed dosage. In some methods, the patient is administered a dose of at least 1.5 mg/kg, at least 2 mg/kg or at least 3 mg/kg, administered once every three weeks or greater.

The dosage administered can vary depending upon known factors, such as the pharmacodynamic characteristics of the particular agent, and its mode and route of administration; the age, health, and weight of the recipient; the type and extent of disease or indication to be treated, the nature and extent of symptoms, kind of concurrent treatment, frequency of treatment, and the effect desired. The initial dosage can be increased beyond the upper level in order to rapidly achieve the desired blood-level or tissue-level. Alternatively, the initial dosage can be smaller than the optimum, and the daily dosage may be progressively increased during the course of treatment.

The frequency of administration depends on the half-life of the ABP or ADC in the circulation, the condition of the patient and the route of administration among other factors. The frequency can be, for example, daily, weekly, monthly, quarterly, or at irregular intervals in response to changes in the patient's condition or progression of the cancer being treated. An exemplary frequency for intravenous administration is between twice a week and quarterly over a continuous course of treatment, although more or less frequent dosing is also possible. Other exemplary frequencies for intravenous administration are weekly, every other week, three out of every four weeks, or every three weeks, over a continuous course of treatment, although more or less frequent dosing is also possible. For subcutaneous administration, an exemplary dosing frequency is daily to monthly, although more or less frequent dosing is also possible.

The number of dosages administered depends on the nature of the cancer (e.g., whether presenting acute or chronic symptoms) and the response of the disorder to the treatment. In some aspects, for acute disorders or acute exacerbations of a chronic disorder between 1 and 10 doses are often sufficient. Sometimes a single bolus dose, optionally in divided form, is sufficient for an acute disorder or acute exacerbation of a chronic disorder. Treatment can be repeated for recurrence of an acute disorder or acute exacerbation. For chronic disorders, an antibody can be administered at regular intervals, e.g., weekly, fortnightly, monthly, quarterly, every six months for at least 1, 5 or 10 years, or the life of the patient.

Exemplary cancers suitable for treatment with the antigen binding proteins provided herein are those that possess ALPP and/or ALPPL2 expression in a cancerous cell or tissue. Examples of cancers that can be treated with an ABP or conjugate thereof include, but are not limited to, hematopoietic tumors, hematopoietic tumors that give rise to solid tumors, solid tumors, soft tissue tumors, and metastatic lesions.

Exemplary solid tumors that can be treated include, but are not limited to, malignancies, e.g., adenocarcinomas, and carcinomas, of the various organ systems, such as those affecting head and neck (including pharynx), lung (small cell lung carcinoma (SCLC) or non-small cell lung carcinoma (NSCLC)), breast, gastrointestinal tract (e.g., oral, esophageal, stomach, liver, pancreas, small intestine, colon and rectum, anal canal), genitals and genitourinary tract (e.g., renal, urothelial, bladder, ovarian, uterine, cervical, endometrial, prostate, testicular), skin (e.g., melanoma) and the like. In certain embodiments, the solid tumor is an NMDA receptor positive teratoma. In other embodiments, the cancer is selected from breast cancer, colon cancer, pancreatic cancer (e.g., a pancreatic neuroendocrine tumors (PNET) or a pancreatic ductal adenocarcinoma (PDAC)), stomach cancer, uterine cancer, and ovarian cancer. In some embodiments, the cancer is a malignant testicular germ cell tumor (GCT) or a malignant ovarian GCT. In further embodiments, the cancer is not a pure teratoma. In some embodiments, the solid tumor cancer is metastatic. In some embodiments, the slid tumor cancer cannot be removed with surgery (unresectable).

In certain embodiments, the cancer is a solid tumor that is associated with ascites. Ascites is a symptom of many types of cancer and can also be caused by a number of conditions, such as advanced liver disease. The types of cancer that are likely to cause ascites include, but are not limited to, cancer of the breast, lung, large bowel (colon), stomach, pancreas, ovary, uterus (endometrium), peritoneum and the like. In some embodiments, the solid tumor associated with ascites is selected from breast cancer, colon cancer, pancreatic cancer, stomach, uterine cancer, and ovarian cancer. In some embodiments, the cancer is associated with pleural effusions, e.g., lung cancer.

In particular embodiments, the cancer is HGSOC wherein the HGSOC has progressed or relapsed in the patient within six months after previous platinum containing chemotherapy, and the patient had received one to three prior anticancer lines of therapy including at least one line of therapy containing bevacizumab or a biosimilar to bevacizumab. In other embodiments, the cancer is NSCLC wherein the patient has unresectable locally advanced or metastatic NSCLC and has received platinum-based therapy and a PD-L1 inhibitor. In other embodiments, the cancer is gastric cancer, wherein the patient has unresectable locally advanced or metastatic gastric cancer and has received prior platinum and fluoropyrimidine-based chemotherapy.

In particular embodiments, the cancer is ovarian cancer, lung cancer, endometrial cancer, bladder cancer, or gastric cancer.

### B. Combination Therapies

The methods, the antigen binding proteins and conjugates described herein can be used in combination with other therapeutic agents and/or modalities. In such combination therapeutic methods, two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive (i.e., a synergistic response). The delivery can be such that an effect of the first treatment delivered is still detectable when the second is delivered.

In certain embodiments, the methods provided herein include administering to the subject an ABP (e.g., an anti-ALPP/ALPPL2 antibody) or ADC as described herein, e.g., a composition or preparation, in combination with one or more additional therapies, e.g., surgery, radiation therapy, or administration of another therapeutic preparation. For example, in some embodiments, the ABP is combined with chemotherapy (e.g., a cytotoxic agent), a targeted therapy (e.g, an antibody against a cancer antigen), an angiogenesis inhibitor, and/or an immunomodulatory agent, such as an inhibitor of an immune checkpoint molecule. In other embodiments, the additional therapy is an anti-inflammatory (e.g., methotrexate), or an anti-fibrotic agent. The ABP (e.g, an anti-ALPP/ALPPL2 antibody) or ADC and the additional therapy can be administered simultaneously or sequentially.

Exemplary cytotoxic agents that can be used in combination with the ABP in some embodiments include anti-microtubule agents, topoisomerase inhibitors, antimetabolites, protein synthesis and degradation inhibitors, mitotic inhibitors, alkylating agents, platinating agents, inhibitors of nucleic acid synthesis, histone deacetylase inhibitors (HDAC inhibitors, e.g., vorinostat (SAHA, MK0683), entinostat (MS-275), panobinostat (LBH589), trichostatin A (TSA), mocetinostat (MGCD0103), belinostat (PXD101), romidepsin (FK228, depsipeptide)), DNA methyltransferase inhibitors, nitrogen mustards, nitrosoureas, ethylenimines, alkyl sulfonates, triazenes, folate analogs, nucleoside analogs, ribonucleotide reductase inhibitors, vinca alkaloids, taxanes, epothilones, intercalating agents, agents capable of interfering with a signal transduction pathway, agents that promote apoptosis and radiation, or antibody molecule conjugates that bind surface proteins to deliver a toxic agent. In one embodiment, the cytotoxic agent that can be administered with an ABP described herein is a platinum-based agent (such as cisplatin), cyclophosphamide, dacarbazine, methotrexate, fluorouracil, gemcitabine, capecitabine, hydroxyurea, topotecan, irinotecan, azacytidine, vorinostat, ixabepilone, bortezomib, taxanes (e.g., paclitaxel or docetaxel), cytochalasin B, gramicidin D, ethidium bromide, emetine, mitomycin, etoposide, tenoposide, vincristine, vinblastine, vinorelbine, colchicin, anthracyclines (e.g., doxorubicin or epirubicin) daunorubicin, dihydroxy anthracin dione, mitoxantrone, mithramycin, actinomycin D, adriamycin, 1-dehydrotestosterone, glucocorticoids, procaine, tetracaine, lidocaine, propranolol, puromycin, ricin, or maytansinoids.

In some embodiments, the antigen binding protein is administered as part of a chemotherapeutic regimen such as CHOP (cyclophosphamide, doxorubicin, vincristine, and prednisone); CVP (cyclophosphamide, vincristine, and prednisone); RCVP (rituximab+CVP); RCHOP (rituximab+CHOP); RCHP (rituximab, cyclophosphamide, doxorubicin, and prednisone); RICE (Rituximab+ifosamide, carboplatin, etoposide); RDHAP, (Rituximab+dexamethasone, cytarabine, cisplatin); RESHAP (rituximab+etoposide, methylprednisolone, cytarabine, cisplatin); R-BENDA (rituximab and Bendamustine), RGDP (rituximab, gemcitabine, dexamethasone, cisplatin). In an embodiment, one of CHOP, CVP, RCVP, RCHOP, RCHP, RICE, RDHAP, RESHAP, R-BENDA, and RGDP is administered in a combination therapy with an antigen binding protein or conjugate as described herein.

Examples of targeted therapies that can be combined with an ABP in certain embodiments include, but are not limited to, use of therapeutic antibodies. Exemplary antibodies include, but are not limited to, those which bind to cell surface proteins such as Her2, CDC20, CDC33, mucin-like glycoprotein, and epidermal growth factor receptor (EGFR) present on tumor cells, and optionally induce a cytostatic and/or cytotoxic effect on tumor cells displaying these proteins. Exemplary antibodies also include HERCEPTIN® (trastuzumab), which may be used to treat breast cancer and other forms of cancer, and RITUXAN® (rituximab), ZEVALIN® (ibritumomab tiuxetan), GLEEVEC® and LYMPHOCIDE® (epratuzumab), which may be used to treat non-Hodgkin's lymphoma and other forms of cancer. Other exemplary antibodies include panitumumab (VECTIBIX®), ERBITUX® (IMC-C225); ertinolib (Iressa); BEXXAR® (iodine 131 tositumomab); KDR (kinase domain receptor) inhibitors; anti VEGF antibodies and antagonists (e.g., Avastin®, motesanib, and VEGAF-TRAP); anti VEGF receptor antibodies and antigen binding regions; anti-Ang-1 and Ang-2 antibodies and antigen binding regions; antibodies to Tie-2 and other Ang-1 and Ang-2 receptors; Tie-2 ligands; antibodies against Tie-2 kinase inhibitors; inhibitors of Hif-1a, and Campath® (Alemtuzumab). In certain embodiments, cancer therapy agents are polypeptides which selectively induce apoptosis in tumor cells, including, but not limited to, the TNF-related polypeptide TRAIL.

In certain embodiments, an antigen binding protein as provided herein is used in combination with one or more anti-angiogenic agents that decrease angiogenesis. Such agents include, but are not limited to, IL-8 antagonists; Campath®, B-FGF; FGF antagonists; Tek antagonists (Cerretti et al., U.S. Publication No. 2003/0162712; Cerretti et al., U.S. Pat. No. 6,413,932, and Cerretti et al., U.S. Pat. No. 6,521,424); anti-TWEAK agents (which include, but are not limited to, antibodies and antigen binding regions); soluble TWEAK receptor antagonists (Wiley, U.S. Pat. No. 6,727,225); an ADAM distintegrin domain to antagonize the binding of integrin to its ligands (Fanslow et al., U.S. Publication No. 2002/0042368); anti-eph receptor and anti-ephrin antibodies, antigen binding regions, or antagonists (U.S. Pat. Nos. 5,981,245; 5,728,813; 5,969,110; 6,596,852; 6,232,447; 6,057,124); anti-VEGF agents (e.g., antibodies or antigen binding regions that specifically bind VEGF, or soluble VEGF receptors or a ligand binding regions thereof) such as Avastin® or VEGF-TRAP™, and anti-VEGF receptor agents (e.g., antibodies or antigen binding regions that specifically bind thereto), EGFR inhibitory agents (e.g., antibodies or antigen binding regions that specifically bind thereto) such as panitumumab, IRESSA® (gefitinib), TARCEVA® (erlotinib), anti-Ang-1 and anti-Ang-2 agents (e.g., antibodies or antigen binding regions specifically binding thereto or to their receptors, e.g., Tie-2/TEK), and anti-Tie-2 kinase inhibitory agents (e.g., antibodies or antigen binding regions that specifically bind and inhibit the activity of growth factors, such as antagonists of hepatocyte growth factor (HGF, also known as Scatter Factor), and antibodies or antigen binding regions that specifically bind its receptor “c-met”; anti-PDGF-BB antagonists; antibodies and antigen binding regions to PDGF-BB ligands; and PDGFR kinase inhibitors.

Other anti-angiogenic agents that can be used in combination with an antigen binding protein include agents such as MMP-2 (matrix-metalloproteinase 2) inhibitors, MMP-9 (matrix-metalloproteinase 9) inhibitors, and COX-II (cyclooxygenase II) inhibitors. Examples of useful COX-II inhibitors include CELEBREX® (celecoxib), valdecoxib, and rofecoxib.

An “immune checkpoint molecule,” as used herein, refers to a molecule in the immune system that either turns up a signal (a stimulatory molecule) and/or turns down a signal (an inhibitory molecule). Many cancers evade the immune system by inhibiting T cell signaling. Exemplary immune checkpoint molecules that can be used with an ABP in certain embodiments include, but are not limited to, programmed cell death protein 1 (PD-1), programmed death-ligand 1 (PD-L1), PD-L2, cytotoxic T lymphocyte-associated protein 4 (CTLA-4), T cell immunoglobulin and mucin domain containing 3 (TIM-3), lymphocyte activation gene 3 (LAG-3), carcinoembryonic antigen related cell adhesion molecule 1 (CEACAM-1), CEACAM-5, V-domain Ig suppressor of T cell activation (VISTA), B and T lymphocyte attenuator (BTLA), T cell immunoreceptor with Ig and ITIM domains (TIGIT), leukocyte-associated immunoglobulin-like receptor 1 (LAIR1), CD160, TGFR, adenosine 2A receptor (A2AR), B7-H3 (also known as CD276), B7-H4 (also called VTCN1), indoleamine 2,3-dioxygenase (IDO), 2B4, killer cell immunoglobulin-like receptor (KIR), OX40, 4-1BB, 4-1BBL, B7-H3, Inducible T-cell Co-stimulator (ICOS/ICOS-L), CD27/CD70, Glucocorticoid-Induced TNF Receptor (GITR), CD47/Signal-Regulatory Protein alpha (SIRPα), and Indoleamine-2,3-Dioxygenase (IDO).

Specific examples of immune checkpoint inhibitors that can be used in combination with the ABP in certain embodiments include, but are not limited to, the following monoclonal antibodies: PD-1 inhibitors such as pembrolizumab (Keytruda®, Merck) and nivolumab (Opdivo®, Bristol-Myers Squibb); PD-L1 inhibitors such as atezolizumab (Tecentriq®, Genentech), avelumab (Bavencio®, Pfizer), durvalumab (Imfinzi®, AstraZeneca); and CTLA-1 inhibitors such as ipilimumab (Yervoy®, Bristol-Myers Squibb) and tremelimumab (AstraZeneca).

### VI. Diagnostic Applications

In another aspect, the ABP (e.g, an anti-ALPP/ALPPL2 antibody or fragment thereof), polypeptides, and nucleic acids as provided herein can be used in methods for detecting, diagnosing and monitoring of a disease, disorder or condition associated with the ALPP and/or ALPPL2.

In some embodiments, the method comprises detecting the expression of ALPP and/or ALPPL2 in a sample obtained from a subject suspected of having a disorder associated with ALPP and/or ALPPL2. In some embodiments, the method of detection comprises contacting the sample with an antibody, polypeptide, or polynucleotide as described herein and determining whether the level of binding differs from that of a reference or comparison sample. In some embodiments, such methods are useful to determine whether the antibodies or polypeptides described herein are an appropriate treatment for the subject.

For example, in one embodiment, the cells or cell/tissue lysate are contacted with an anti-ALPP/ALPPL2 antibody and the binding between the antibody and the cell or antigen is determined. When the test cells show binding activity as compared to a reference cell of the same tissue type, it may indicate presence of a disease or condition associated with ALPP and/or ALPPL2. In some embodiments, the test cells are from human tissues.

Various methods known in the art for detecting specific antibody-antigen binding can be used. Exemplary immunoassays which can be conducted according to the invention include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA).

Diagnostic applications provided herein include use of an ABP (e.g, an anti-ALPP/ALPPL2 antibody or fragment thereof) to detect expression of ALPP and/or ALPPL2 and binding of the ligands to ALPP and/or ALPPL2. For diagnostic applications, the ABP typically is labeled with a detectable labeling group. Suitable labeling groups include, but are not limited to, the following: radioisotopes or radionuclides (e.g., ³H, ¹⁴C, ¹⁵N, ³⁵S ⁹⁰Y, ⁹⁹Tc, ¹¹¹In, ¹²⁵I, ¹³¹I), fluorescent groups (e.g., FITC, rhodamine, lanthanide phosphors), enzymatic groups (e.g., horseradish peroxidase, β-galactosidase, luciferase, alkaline phosphatase), chemiluminescent groups, biotinyl groups, or predetermined polypeptide epitopes recognized by a secondary reporter (e.g., leucine zipper pair sequences, binding sites for secondary antibodies, metal binding domains, epitope tags). In some embodiments, the labeling group is coupled to the ABP via spacer arms of various lengths to reduce potential steric hindrance. Various methods for labeling proteins are known in the art and may be used. Examples of methods useful in the detection of the presence of ALPP and/or ALPPL2 include immunoassays such as those described above.

In another aspect, an ABP can be used to identify a cell or cells that express ALPP and/or ALPPL2. In a specific embodiment, the antigen binding protein is labeled with a labeling group and the binding of the labeled antigen binding protein to ALPP and/or ALPPL2 is detected. In a further specific embodiment, the binding of the antigen binding protein to ALPP and/or ALPPL2 is detected in vivo.

An antigen binding protein (e.g., an anti-ALPP/ALPPL2 antibody or fragment thereof) also can be used as staining reagent in pathology using techniques well known in the art.

### VIII. Pharmaceutical Compositions and Formulations

Pharmaceutical compositions that comprise an ABP (e.g., an anti-ALPP/ALPPL2 antibody or fragment thereof) are also provided and can be utilized in any of the therapeutic applications disclosed herein. In certain embodiments, the pharmaceutical composition comprises a therapeutically effective amount of one or a plurality of the antigen binding protein, together with pharmaceutically acceptable diluent or carrier. In other embodiments, the pharmaceutical composition comprises a therapeutically effective amount of one or a plurality of the antigen binding proteins, a pharmaceutically acceptable diluent, carrier, solubilizer, emulsifier, preservative, and/or adjuvant. Acceptable formulation materials are nontoxic to recipients at the dosages and concentrations employed. The pharmaceutical compositions can be formulated as liquid, frozen or lyophilized compositions.

In certain embodiments, the pharmaceutical composition can contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids; antimicrobials; antioxidants; buffers; bulking agents; chelating agents; complexing agents; fillers; carbohydrates such as monosaccharides or disaccharides; proteins; coloring, flavoring and diluting agents; emulsifying agents; hydrophilic polymers; low molecular weight polypeptides; salt-forming counterions (such as sodium); preservatives; solvents (such as glycerin, propylene glycol or polyethylene glycol); sugar alcohols; suspending agents; surfactants or wetting agents; stability enhancing agents; tonicity enhancing agents; delivery vehicles; and/or pharmaceutical adjuvants. Additional details and options for suitable agents that can be incorporated into pharmaceutical compositions are provided in, for example, Remington's Pharmaceutical Sciences, 22^{nd} Edition, (Loyd V. Allen, ed.) Pharmaceutical Press (2013); Ansel et al., Pharmaceutical Dosage Forms and Drug Delivery Systems, 7^{th} ed., Lippencott Williams and Wilkins (2004); and Kibbe et al., Handbook of Pharmaceutical Excipients, 3^{rd} ed., Pharmaceutical Press (2000).

The components of the pharmaceutical composition are selected depending upon, for example, the intended route of administration, delivery format and desired dosage. See, for example, Remington's Pharmaceutical Sciences, 22^{nd} Edition, (Loyd V. Allen, ed.) Pharmaceutical Press (2013). The compositions are selected to influence the physical state, stability, rate of in vivo release and rate of in vivo clearance of the antigen binding proteins disclosed. The primary vehicle or carrier in a pharmaceutical composition can be either aqueous or non-aqueous in nature. For example, a suitable vehicle or carrier can be water for injection or physiological saline solution. In certain embodiments, antigen binding protein compositions can be prepared for storage by mixing the selected composition having the desired degree of purity with optional formulation agents in the form of a lyophilized cake or an aqueous solution. Further, in certain embodiments, the antigen binding protein can be formulated as a lyophilizate using appropriate excipients.

A pharmaceutical composition is formulated to be compatible with its intended route of administration. Examples of routes of administration are intravenous (IV), intradermal, inhalation, transdermal, topical, transmucosal, and rectal administration. A preferred route of administration for an antigen binding protein (e.g, an antibody) is IV infusion. In another preferred embodiment, the preparation is administered by intramuscular or subcutaneous injection.

### IX. Kits/Articles of Manufacture

Kits containing an ABP as described herein are also provided. In one embodiment, such kits comprise one or more containers comprising an antigen binding protein (e.g, an anti-ALPP/ALPPL2 antibody), or unit dosage forms and/or articles of manufacture. In some embodiments, a unit dosage is provided wherein the unit dosage contains a predetermined amount of a composition comprising an antigen binding protein, with or without one or more additional agents. In some embodiments, such a unit dosage is supplied in a single-use prefilled syringe for injection. In various embodiments, the composition contained in the unit dosage may comprise: saline; a buffer, other formulation components, and/or be formulated within a stable and effective pH range as described herein. Alternatively, in some embodiments, the composition is provided as a lyophilized powder that can be reconstituted upon addition of an appropriate liquid, for example, sterile water.

Some kits as provided herein further comprise instructions for use in the treatment of a disease associated with ALPP and/or ALPPL2, such as ovarian cancer in accordance with any of the methods described herein. The kit can further comprise a description of how to select or identify an individual suitable for treatment. Instructions supplied in the kits of the invention are typically written instructions on a label or package insert (e.g., a paper sheet included in the kit), but machine-readable instructions (e.g., instructions carried on a magnetic or optical storage disk) are also acceptable. In some embodiments, the kit further comprises another therapeutic agent, such as those described above as suitable for use in combination with the antigen binding protein.

In a further aspect, kits for detecting the presence of ALPP and/or ALPPL2, or a cell expressing ALPP and/or ALPPL2, in a sample are provided. Such kits typically comprise an antigen binding protein as described herein and instructions for use of the kit.

Certain kits, for example, are for diagnosis of cancer and comprises a container comprising an antigen binding protein (e.g, an anti-ALPP/ALPPL2 antibody), and one or more reagents for detecting binding of the antigen binding protein to ALPP and/or ALPPL2. Such reagents can include, for example, fluorescent tags, enzymatic tags, or other detectable tags. The reagents can also include secondary or tertiary antibodies or reagents, e.g., for use in enzymatic reactions that produce a product that can be visualized. In one embodiment, a diagnostic kit comprises one or more antigen binding proteins in labeled or unlabeled form in suitable container(s), reagents for the incubations for an indirect assay, and substrates or derivatizing agents for detection in such an assay, depending on the nature of the label.

Kits such as provided herein can be used for in situ detection. Some methods utilizing such kits comprise removing a histological specimen from a patient and then combining the labeled antigen binding protein (e.g, an anti-ALPP/ALPPL2 antibody) with the biological sample. With such methods, it is possible to determine not only the presence of ALPP or ALPP-fragments and/or ALPPL2 or ALPPL2-fragments but also the distribution of such peptides in the examined tissue (e.g., in the context of assessing the spread of cancer cells).

In another aspect, an anti-idiotypic antibody (Id) which binds to an antigen binding protein (e.g, an anti-ALPP/ALPPL2 antibody) is provided. An Id antibody can be prepared by immunizing an animal of the same species and genetic type as the source of an anti-ALPP/ALPPL2 mAb with the mAb to which an anti-Id is being prepared. The immunized animal typically can recognize and respond to the idiotypic determinants of the immunizing antibody by producing an antibody to these idiotypic determinants (the anti-Id antibody).

The following examples, including the experiments conducted and the results achieved, are provided for illustrative purposes only and are not to be construed as limiting the scope of the appended claims.

### X. EXAMPLES

### Example 1: ALPP/ALPPL2 Expression Levels

Cell lines described in the following examples were maintained in culture according to the conditions specified by the American Type Culture Collection (ATCC) or Deutsche Sammlung von Mikroorganismen und Zellkulturen GmbH, Braunschweig, Germany (DMSZ), Japanese Cancer Research Resources Bank (JCRB) or as otherwise known.

Quantification of ALPP copy number on the cell surface of various cancer cell lines was determined using a murine ALPP mAb as primary antibody and the DAKO QiFiKit flow cytometric indirect assay as described by the manufacturer (DAKO A/S, Glostrup, Denmark) and evaluated with an Attune NxT Flow Cytometer. The resulting number of ALPP molecules expressed per cell are shown in Table 3. ALPP/ALPPL2 mRNA expression levels were obtained from Genentech cell line RNA-seq data (See Klijn C, et al. Nat Biotechnol. 2015 March; 33(3):306-12).

TABLE 3
ALPP/ALPPL2 molecules per cell for various cell lines

| | | Receptor # | ALPP mRNA | ALPPL2 mRNA |
|---|---|---|---|---|
| Cell line | Indication | (×10³) | (TPM + 1) | (TPM + 1) |
| HEP2 | Cervical | 745 | 849 | 178 |
| NCI-H1651 | Lung | 500 | 165 | 665 |
| RMUGS | Ovarian | 400 | 81 | 16 |
| MKN1 | Gastric | 374 | 148 | 26 |
| COV644 | Ovarian | 200 | 121 | 26 |
| NUGC3 | Gastric | 191 | 58 | 11 |
| NCI-N87 | Gastric | 140 | 41 | 25 |
| CAOV3 | Ovarian | 60 | 12 | 3 |
| CasKi | Cervical | 52 | 48 | 8 |
| LoVo | Colon | 50 | 10 | 6 |
| 647V | Bladder | 33 | 0 | 0 |
| ABC1 | Lung | 31 | 25 | 9 |
| HCC15 | Lung | 0 | 0 | 10 |
| NCI-H1869 | Lung | 0 | 0 | 0 |

Tumor tissue arrays were obtained from commercial sources. Frozen or formalin fixed and paraffin embedded (FFPE) tissues were purchased from US Biomax Inc. All samples were processed on Bond-Max™ autostainer (Leica)

Frozen samples sectioned on glass slides were fixed with acetone for 10 minutes prior to staining. Slides were incubated with the primary anti-ALPP/ALPPL2 antibody (H17E2: Thermo; cat #MA1-20245). Isotype-matched mouse IgG1 was used as negative control for background staining. For automated IHC staining we used a Refine DAB kit (Leica, cat #DS9800). Slides were incubated with mouse monoclonal primary antibodies against ALPP for 45 min at 5 μg/ml with a preliminary incubation with PeroxAbolish (Biocare Medical cat #PXA969M) reagent for 15 minutes and 20 min protein block (DAKO cat #X0909). FFPE slides sectioned on glass slides were de-paraffinized using Bond™ Dewax solution (Leica, cat #AR9222) at 72° C. and rehydrated. Antigen retrieval was performed using EDTA based Bond™ Epitope Retrieval Solution 2 (Leica, cat #AR9640) for 20 min at 95-100° C. before incubation with the primary anti-ALPP/ALPPL2 antibody (25C3 monoclonal Ab; in-house developed mouse monoclonal) for 45 min at 1 μg/ml. Isotype-matched mouse IgG2a was used as negative control for background staining. For automated IHC staining we used a Refine DAB kit (Leica, cat #DS9800), Slides were incubated with mouse monoclonal antibodies against ALPP mAb for 45 min at 1 μg/ml with a preliminary 20 min protein block (DAKO cat #X0909) After chromogen development, sections were counterstained with hematoxylin and coverslipped. Slides were evaluated and scored by a pathologist and images were taken using an Aperio slide scanner (Leica). Staining intensity was score from 0 to +3 and the frequency was in quartiles (0-25, 26-50, 51-75 and 76-100). ALPP/ALPPL2 expression prevalence was found to be high in multiple solid tumor indications including ovarian, testicular and endometrial as it is shown in Table 4. ˜25% of Lung adenocarcinoma, gastric and bladder cancer samples also present ALPP/ALPPL2 expression.

TABLE 4

| | Any | High |
|---|---|---|
| Cancer | Expression^{†} | Expression ^{††} |
| Ovarian¹ | 90% | 70% |
| Testicular | 80% | 60% |
| Endometrial | 57% | 41% |
| Lung adenocarcinoma | 80%* | 25% |
| (NSCLC) | | |
| Stomach | 30% | 25% |
| Bladder² | 59% | 23% |

¹Platinum resistant
²unresectable
^{†}Incidence based on any frequency and intensity
^{††} Expression frequency based on score 2+, >25% of positive cells
*1+ expression noted at interface w/non-neoplastic tissues.

### Example 2: Lead Antibody Selection

### Conjugation and In Vitro Cytotoxicity

Mice were immunized with recombinant full-length ALPPL2. Lymphocytes harvested from spleen and lymph nodes of ALPP antibody producing mice were fused to myeloma cells. Fused cells were recovered overnight in hybridoma growth media. Following recovery, cells were spun down and then plated in semi-solid media. Hybridomas were incubated and IgG producing hybridoma clones were picked. Antibodies from this hybridoma campaign were screened on ALPP, ALPPL2, ALPI and ALPL-expressing HEK293 cell lines using the iQue according to the manufacturer instructions. Antibodies with cross-reactivity to ALPP and ALPPL2, but not ALPI and ALPL, were evaluated as ADCs.

Various mouse anti-ALPP/ALPPL2 monoclonal mouse antibodies were conjugated to 10-12 loads of either MDpr-PEG(12)-gluc-MMAE or Auristatin T, which either exhibit bystander activity or do not, respectively. The conjugation method is described in U.S. Publ. No. 2018/0092984.

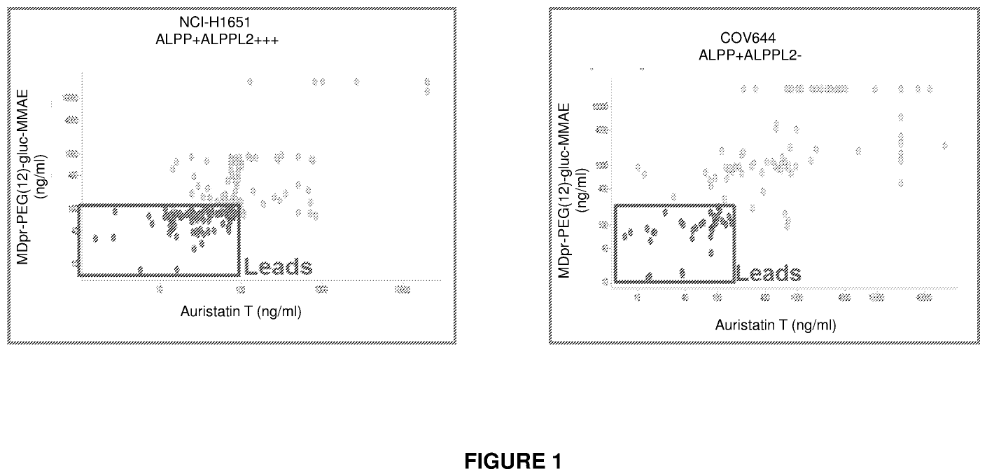

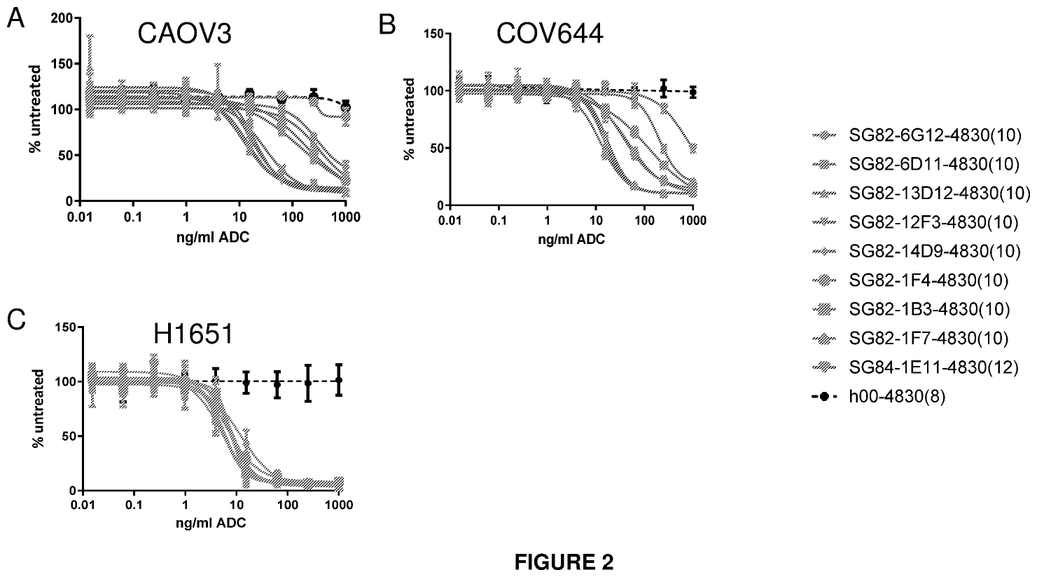

CAOV3 (ALPP), COV644 (ALPP+) and NCI-H1651 (ALPPL2++ALPP+) Tumor cells were incubated with ALPP/ALPPL2 antibody drug conjugates (ADCs) for 96 hours at 37° C. A human IgG ADC was used as a negative control. Cell viability was measured using Cell Titer Glo according to manufacturer's instructions. Fluorescent signal was measured on a Fusion HT fluorescent plate reader (Perkin Elmer, Waltham, MA). The data was normalized to untreated cells, and ×50 values were calculated using Graph Pad software. As shown in FIGS. 1-2, a sub-group of Abs exhibited low ×50 values with both payloads suggesting high drug delivery capabilities.

### Flow Cytometry and Saturation Binding Assays

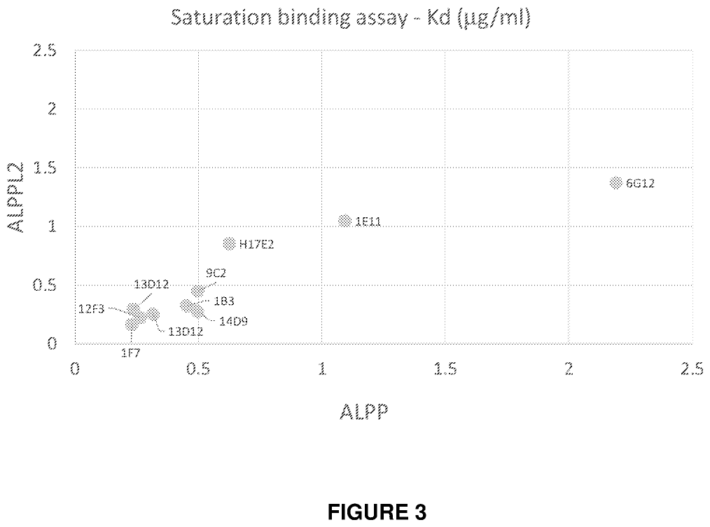

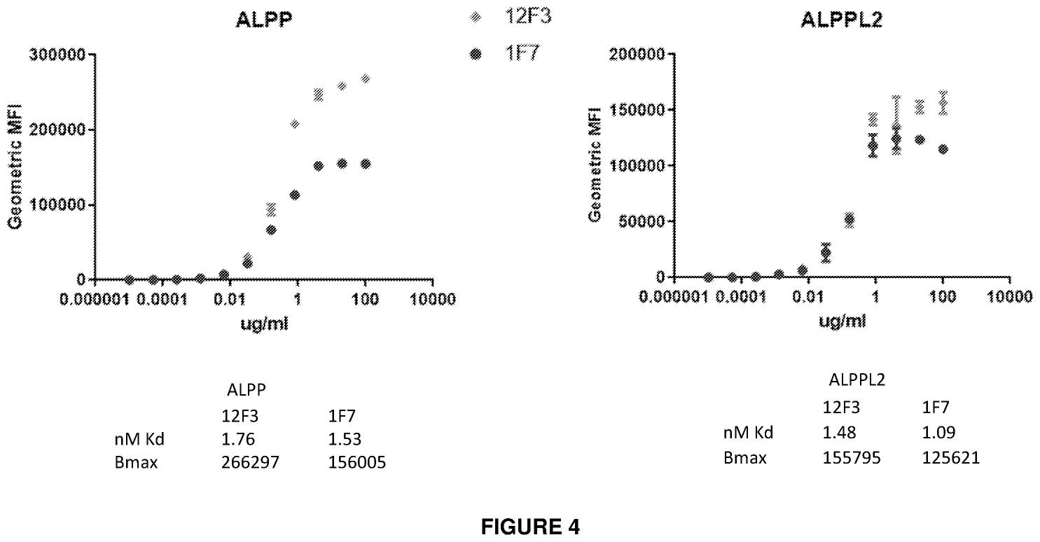

Cynomolgus ALPP, human ALPP, ALPPL2, ALPI and ALPL-expressing HEK293 cells were used to evaluate specificity and binding affinity. Briefly, one hundred thousand target-expressing HEK293 cells were transferred to 96-well plates. Cells were pelleted by centrifugation and resuspended in 100 μL of PBS+2% w/v BSA. After blocking, cells were resuspended in PBS+2% w/v BSA with unlabeled monoclonal anti-ALPP/ALPPL2 antibodies ranging in concentration from 8 pM to 666 nM and incubated on ice for 30 minutes. Cells were washed twice in PBS and resuspended in R-PE labeled secondary goat anti-human or anti-mouse antibodies (Jackson Immunoresearch, West Grove, PA) for 30 minutes on ice. Specificity of monoclonal antibodies for human and cynomolgus ALPP and ALPPL2, but not other members of this alkaline phosphatase family was confirmed by flow cytometry. Fluorescence was analyzed using an Attune NxT flow cytometer, and the percent of saturated fluorescent signal was used to determine percent bound and to subsequently calculate apparent K_{D}. The antibodies 1C7 and 12F3 showed the lowest K_{D} among the top candidates as shown in FIG. 3. However, while SG82-12F3 and SG84-1F7 show similar affinity for their targets, SG82-12F3 shows higher saturation levels for ALPP than SG84-1F7, as shown in FIG. 4. Ultimately, the 12F3 antibody was selected for humanization based on its superior ADC cytotoxicity and high affinity for ALPP/ALPPL2, as well as having an epitope that is conserved with the cynomogus ortholog.

### Example 3: Humanization and Binding Studies

Humanized antibodies were derived from the murine 12F3 antibody. Eight humanized heavy chains (HA-HH) and twelve humanized light chains (L1-LI) were made incorporating back mutations at different positions. In some instances, back mutations matched the murine germline, in other cases it did not (as in the case with somatic mutations). Humanized heavy and light chains were paired. See, FIGS. 5-8 for the sequence alignments and Tables 5-8 for the specific mutations made.

TABLE 5
Humanizing Mutations in h12F3 Variable Heavy (vH) Chain Variants

| | Human Heavy | Murine Donor | Human | Secondary Human V-Gene |
|---|---|---|---|---|
| vH | Acceptor | Framework | Acceptor CDR | Acceptor Residues |
| Variant | Sequence | Residues | Residues | (IGHV3-72) |
| hvHA | IGHV3-49/HJ4 | none | none | None |
| hvHB | IGHV3-49/HJ4 | H30, H73 | none | None |
| hvHC | IGHV3-49/HJ4 | H30, H48, H49, H73 | none | None |
| hvHD | IGHV3-49/HJ4 | H30, H73, H78, H93 | none | None |
| hvHE | IGHV3-49/HJ4 | H30, H48, H49, H73, | none | None |
| | | H78, H93 | | |
| hvHF | IGHV3-49/HJ4 | H30, H37, H48, H49, | none | None |
| | | H73, H78, H93 | | |
| hvHG | IGHV3-49/HJ4 | H30, H37, H48, H49, | H60 | None |
| | | H73, H78, H93 | | |
| hvHH | IGHV3-49/HJ4 | H30, H37, H48, H49, | H60 | H76, H77 |
| | | H73, H78, H93 | | |

TABLE 6
Specific Murine Framework Mutations in
h12F3 Variable Heavy Chain Variants

| vH Variant | 30 | 37 | 48 | 49 | 73 | 78 | 93 | % Human |
|---|---|---|---|---|---|---|---|---|
| hvHA | | | | | | | | 94.0 |
| hvHB | T | | | | N | | | 92.0 |
| hvHC | T | | L | A | N | | | 90.0 |
| hvHD | T | | | | N | L | A | 90.0 |
| hyHE | T | | L | A | N | L | A | 88.0 |
| hvHF | T | V | L | A | N | L | A | 88.0 |
| hvHG | T | V | L | A | N | L | A | 88.0 |
| hvHH | T | V | L | A | N | L | A | 87.0 |

TABLE 7
Humanizing Mutations in h12F3 Variable Kappa (vL) Light Chain Variants

| | Human Kappa | Murine Donor | Human | Secondary Human V-Gene |
|---|---|---|---|---|
| vL | Acceptor | Framework | Acceptor CDR | Acceptor Residues |
| Variant | Sequence | Residues | Residues | (IGKV1D-43, IGKV1-16) |
| hvL1 | IGKV1-33/KJ2 | none | L24, L33, L34, | None |
| | | | L53, L55, L56 | |
| hvL2 | IGKV1-33/KJ2 | none | L24, L33, L34, | L53, L56 |
| | | | L53, L56 | |
| hvL3 | IGKV1-33/KJ2 | none | L24, L33, L53 | L53 |
| hvLA | IGKV1-33/KJ2 | none | none | None |
| hvLB | IGKV1-33/KJ2 | L2, L49, L69 | none | L71 |
| hvLC | IGKV1-33/KJ2 | L2 | none | L71 |
| hvLD | IGKV1-33/KJ2 | L2 | L24, L53 | L53, L71 |
| hvLE | IGKV1-33/KJ2 | L2, L49, L69 | L24, L53, L56 | L53, L56, L71 |
| hvLF | IGKV1-33/KJ2 | L2, L38, L49, L69 | L24, L33, L53, L56 | L53, L56, L71 |
| hvLG | IGKV1-33/KJ2 | L2, L40, L49, L69 | L24, L33, L53, L56 | L53, L56, L71 |
| hvLH | IGKV1-33/KJ2 | L2, L38, L40, | none | L71 |
| | | L49, L69 | | |
| hvLI | IGKV1-33/KJ2 | L2, L38, L40, | none | L36, L47, L71, L73 |
| | | L49, L69 | | |

TABLE 8
Specific Murine Framework Mutations in h12F3
Variable Kappa Light Chain Variants

| vL Variant | 2 | 38 | 40 | 49 | 69 | % Human |
|---|---|---|---|---|---|---|
| hvL1 | | | | | | 94.7 |
| hvL2 | | | | | | 91.5 |
| hvL3 | | | | | | 90.4 |
| hvLA | | | | | | 88.3 |
| hvLB | T | | | H | R | 84.0 |
| hvLC | T | | | | | 86.2 |
| hvLD | T | | | | | 87.2 |
| hvLE | T | | | H | R | 85.1 |
| hvLF | T | Y | | H | R | 85.1 |
| hvLG | T | | T | H | R | 85.1 |
| hvLH | T | Y | T | H | R | 81.9 |
| hvLI | T | Y | T | H | R | 78.7 |

Antibodies designated HALT (antibody having the heavy chain variable region designated vHA and the light chain variable region designated vL1), HAL2 (antibody having the heavy chain variable region designated vHA and the light chain variable region designated vL2) HAL3 (antibody having the heavy chain variable region designated vHA and the light chain variable region designated vL3), HALA (antibody having the heavy chain variable region designated vHA and the light chain variable region designated vLA), HALB (antibody having the heavy chain variable region designated vHA and the light chain variable region designated vLB), HALC (antibody having the heavy chain variable region designated vHA and the light chain variable region designated vLC), HALD (antibody having the heavy chain variable region designated vHA and the light chain variable region designated vLD), HALE (antibody having the heavy chain variable region designated vHA and the light chain variable region designated vLE), HALE (antibody having the heavy chain variable region designated vHA and the light chain variable region designated vLE), HALF (antibody having the heavy chain variable region designated vHA and the light chain variable region designated vLF), HALG (antibody having the heavy chain variable region designated vHA and the light chain variable region designated vLG), HALH (antibody having the heavy chain variable region designated vHA and the light chain variable region designated vLH), and HALI (antibody having the heavy chain variable region designated vHA and the light chain variable region designated vLI) can be used in the present invention in place of the HGLF antibody. Similarly, antibodies having any permutation of heavy chain variable region designated vHA, vHB, vHC, vHD, vHE, vHF, vHG, or vHH with light chain variable region designated vL1, vL2, vL3, vLA, vLB, vLC, vLD, vLE, vLF, vLG, vLH, or vLI can be used in the present invention in place of the HGLF antibody. See FIGS. 5-8 for the vHA, vHB, vHC, vHD, vHE, vHF, vHG, vHH, vL1, vL2, vL3, vLA, vLB, vLB-Q, vLB-V, vLC, vLD, vLE, vLF, vLG, vLH, and vLI sequences.

Humanized antibodies with low quality, low expression yield or unfavorable sequence were not evaluated on functional assays. The apparent affinity of the humanized antibodies on ALPPL2-expressing cells was estimated using flow cytometry. Briefly, the K_{D} for each resulting antibody was then determined by a saturation binding assay. HEK293 cells stably expressing human ALPPL2 were aliquoted at 1E5 cells per well in 96-well v-bottom plates. Each humanized ALPP/ALPPL2 antibody was added in concentrations from 0.2, 2 and 20 nM and incubated on ice for 60 minutes. Cells were pelleted and washed 2× with PBS/FBS followed by addition of 10 g/ml of an APC labeled anti-human IgG mouse secondary antibody and incubated on ice for an additional 60 minutes. Cells were pelleted and washed 2× with PBS/FBS and resuspended in 100 μL of 2% paraformaldehyde. Fluorescence was analyzed by flow cytometry, using percent of saturated fluorescent signal to determine percent bound and to subsequently calculate apparent K_{D}, based on three antibody concentration. The apparent K_{D} for recombinant humanized anti-ALPP/ALPPL2 were compared to c12F3 (chimeric 12F3 IgG1 k) as it is shown in Table 9.

TABLE 9
Binding of hALPP-1 Antibody Variants by flow cytometry
on HEK-ALPPL2 Cells (KD (nM)); NT = Not tested.

| | L1 | L2 | L3 | LA | LB | LC | LD | LE | LF | LG | LH | LI |
|---|---|---|---|---|---|---|---|---|---|---|---|---|
| HA | NT | NT | NT | NT | NT | NT | NT | NT | NT | NT | NT | NT |
| HB | NT | NT | 1.8 | 1.6 | 1.3 | 3.1 | 2.5 | 1.2 | 1.2 | 1.7 | 1.4 | 2.4 |
| HC | NT | NT | 3.3 | 2.9 | 1.8 | 3.9 | 2.7 | 1.5 | 1.7 | 2.2 | 2.4 | 2.1 |
| HD | NT | NT | 2.0 | 3.1 | 1.4 | 3.0 | 2.1 | 1.7 | 1.6 | 1.9 | 1.9 | 2.9 |
| HE | NT | NT | 3.1 | 3.7 | 1.5 | 4.6 | ~4 | 1.4 | 1.8 | 2.0 | 2.4 | 2.2 |
| HF | NT | NT | 1.1 | 1.2 | 1.1 | 1.8 | 0.7 | 1.3 | 1.4 | 1.3 | 1.4 | 1.5 |
| HG | NT | NT | 1.3 | 1.6 | 1.2 | 1.7 | 1.6 | 1.7 | 1.2 | 1.3 | 1.6 | 1.4 |
| HH | NT | NT | 1.2 | 2.0 | 1.0 | 1.7 | 1.6 | 1.5 | 1.7 | 1.3 | 1.3 | 1.9 |

### Example 4: H12F3 Conjugation and In Vitro Cytotoxicity

Several h12F3 antibodies were constructed using the hIGHV3-49/hIGHJ4 heavy chain variable region human germlines and the hIGKV1-33/hIGKJ2 or hIGKV1D-43/hIGKJ2 or hIGKV1-16/hIGKJ2 light chain variable region human germlines as the human acceptor sequences. The antibodies differed in the selection of amino acid residues to be mutated back to the mouse antibody or mouse germline sequence.

As mentioned above, Humanized antibodies with low quality, low expression or unfavorable sequence were not evaluated on functional assays. For drug delivery evaluation, various humanized versions of the h12F3 antibody were conjugated to 8 loads of MDpr-PEG(12)-gluc-MMAE auristatin T. Upon selection of potential antibody leads, additional cytotoxicity evaluations were performed on different payloads including antibodies conjugated to 4 loads of mc-vc-PABC-MMAE or mp-dLAE-PABC-MMAE or to 8 loads of MDpr-PEG(12)-gluc-MMAE. The conjugation method is described in U.S. Publ. No. 2018/0092984. For mp-dLAE-PABC-MMAE linker conjugation, antibody drug conjugates were prepared as described in PCT/US2020/051648 (filed Sep. 18, 2020) using the humanized anti-ALPP/ALPPL2 antibodies described herein. For antibody conjugation to mp-dLAE-PABC-MMAE, antibody was partially reduced using the appropriate equivalents of TCEP (tris(2-carboxyethyl)phosphine) according to the procedure, which is specifically incorporated by reference herein, of US 2005/0238649. Briefly, the antibody in phosphate buffered saline with 2 mM DTPA, pH 7.4, was treated with 2.1 eq. TCEP and then incubated at 37° C. for about 45 minutes. The thiol/Ab value was checked by reacting the reduced antibody with compound 1 and using hydrophobic interaction chromatography to determine the loading.

The tripeptide-based auristatin drug-linker mp-dLAE-PABC-MMAE compounds were conjugated to the partially reduced antibody using the method, which is specifically incorporated by reference herein, of US 2005/0238649. Briefly, the drug-linker compound (mp-dLAE-PABC-MMME) (50% excess) in DMSO, was added to the reduced antibody in PBS with EDTA along with additional DMSO for a total reaction co-solvent of 10-20%. After 30 minutes at ambient temperature, an excess of QuadraSil MP™ was added to the mixture to quench all unreacted maleimide groups. The resulting ADC was then purified, and buffer exchanged by desalting using Sephadex G25 resin into PBS buffer and kept at −80° C. until further use. The protein concentration of the resulting ADC composition was determined at 280 nm. The drug-antibody ratio (DAR) of the conjugate was determined by hydrophobic interaction chromatography (HIC).

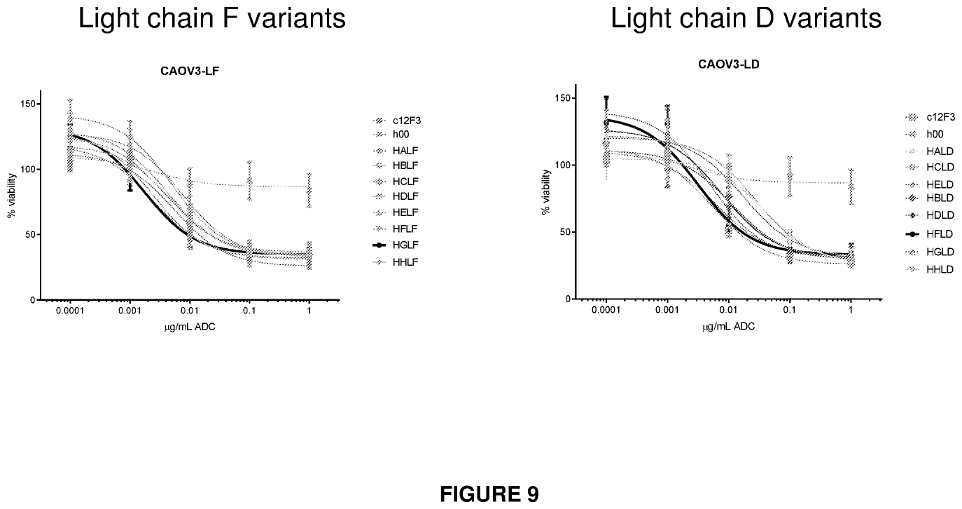

For in vitro cytotoxicity assays, tumor cell lines were plated 24 hours prior to ADC treatment. Cells were treated with the indicated doses of ADC and incubated for 96 hours at 37° C. In some experiments, non-antigen binding ADC was included as a negative control. Cell viability for the cell lines was measured using Cell Titer Glo (Promega Corporation, Madison, WI) according to the manufacturer's instructions. Cells were incubated for 30 minutes at room temperature with the Cell Titer Glo reagents and luminescence was measured on an Envision plate reader (Perkin Elmer, Waltham, MA). As shown in FIG. 9 the humanized version of the h12F3 antibody containing the F variant of the light chain identifies variants with high drug delivery capabilities, especially when compared to other combinations.

Based on the cytotoxic potency and the apparent affinity, selected humanized antibodies were further evaluated for their ability to deliver different payloads to tumor cells. The humanized 12F3 antibodies with high drug delivery capabilities were conjugated to 4 loads of me-vc-MMAE or mc-vc-PABC-MMAE or to 8 loads of MDpr-PEG(12)-gluc MMAE, as described before.

Tumor cells were incubated with each ADC for 96-144 hours at 37° C. A non-binding (referred to as h00 or IgG) ADC was used as a negative control. Cell viability was measured using Cell Titer Glo according to the manufacturer's instructions. Fluorescent signal was measured on a Fusion HT fluorescent plate reader (Perkin Elmer, Waltham, MA). The data was normalized to untreated cells, and IC₅₀ values were calculated using Graph Pad software. Results are reported in Table 10 as IC₅₀, the concentration of compound needed to yield a 50% reduction in viability compared to vehicle-treated cells (control=100%). h12F3 ADCs achieve single- and double-digit ng/ml IC₅₀ values across a panel of cell lines with ALPP expression ranging from 30,000 to 500,000.

TABLE 10
IC50 (ng/ml) of h12F3 HGLF antibody drug conjugates against various cancer cells.
Results are reported as IC50 and remaining viability percentage at the end point.

| | | Receptor # | | | | | MDpr-PEG(12)- | |
|---|---|---|---|---|---|---|---|---|
| | | (×10³) | dLAE-MMAE(4) | | vc-MMAE(4) | | gluc-MMAE(8) | |
| Cell line | Indication | m12F3 Ab | IC50/Viability | | IC50/Viability | | IC50/Viability | |
| Hep2 | Cervical | 745 | 17 | 47 | 18 | 41 | 7 | 2 |
| H1651 | Lung | 500 | 24 | 0 | 12 | 0 | 6 | 0 |
| RMUGS | Ovarian | 400 | 79 | 76 | 27 | 72 | 25 | 54 |
| MKN1 | Gastric | 374 | 12 | 27 | 9 | 25 | 8 | 13 |
| COV644 | Ovarian | 200 | 31 | 70 | 19 | 61 | 18 | 30 |
| NUGC3 | Gastric | 191 | 39 | 17 | 26 | 10 | 5 | 11 |
| CAOV3p1 | Ovarian | 60 | 90 | 72 | 30 | 67 | 18 | 16 |
| CasKi | Cervical | 52 | 13 | 93 | 39 | 81 | 75 | 68 |
| LoVo | Colon | 50 | 6 | 33 | 5 | 27 | 4 | 13 |
| 647V | Bladder | 33 | >1000 | | 286 | 79 | 82 | 88 |
| ABC1 | Lung | 31 | 3 | 83 | >1000 | | 7 | 38 |
| HCC15 | Lung | 0 | >1000 | | >1000 | | >1000 | |

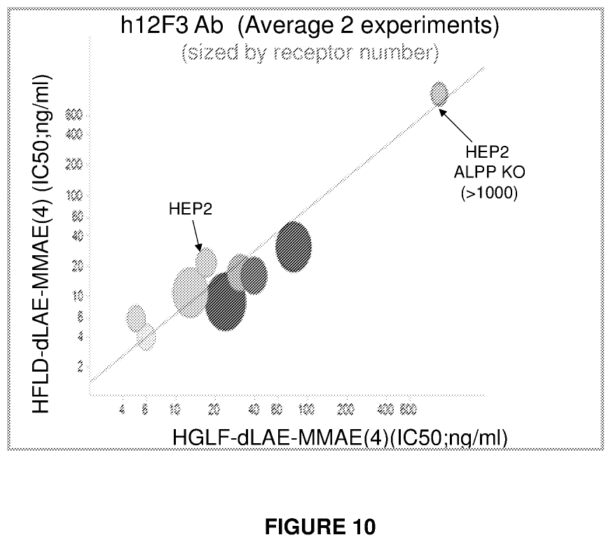

Comparison of cytotoxic potency of antibody drug conjugates for humanized variants using the same payload (mp-dLAE-MMAE) was done by graphing the IC50 values across multiple cell lines. Humanized variants for 12F3 antibody showed similar potency in vitro as it is shown in FIG. 10.

The antibody designated HGLF (heavy chain variable region as set forth in SEQ ID NO:15 (vHG) and the light chain variable region as set forth in SEQ ID NO:30 (vLF)) was ultimately selected as the lead humanized anti-ALPP/ALPPL2 antibody on the basis of its (i) binding characteristics, (ii) ability to deliver drug and (iii) number of back mutations as compared to the other variants (see Tables 5-8).

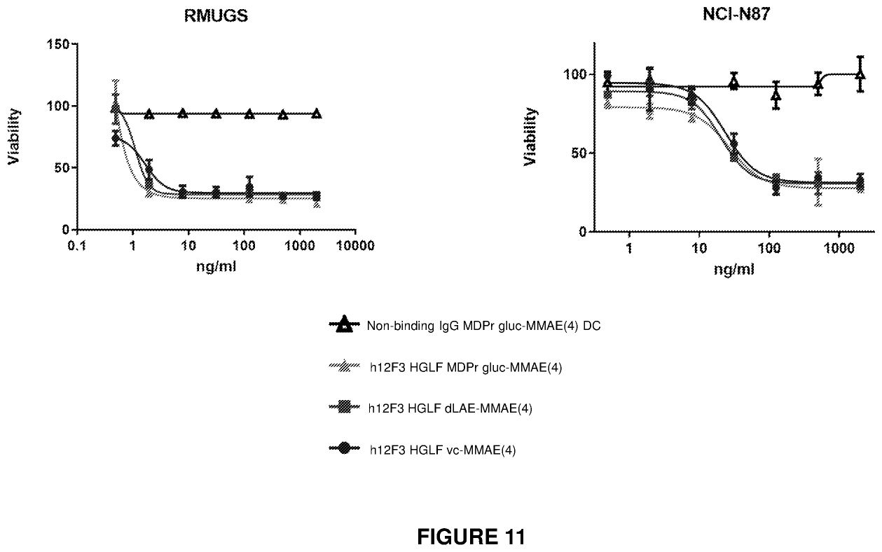

Evaluation of HGLF ADCs on tumor cancer cell spheroids was performed as follows: 100 μL of cells at 2.5E4 cell/well in ultra-low attachment round-bottom 96-well plates (Corning, Corning, NY) for 48 h at 37 C. After this incubation, 100 μL of medium containing 2× of ADC was added and incubated for 120 h at 37 C. In some experiments, non-antigen binding ADC was included as a negative control. Cell viability for the cell lines was measured using 3D Cell Titer Glo (Promega Corporation, Madison, WI) according to the manufacturer's instructions. Cells were incubated for 30 minutes at room temperature with 3D Cell Titer Glo reagent and luminescence was measured on an Envision plate reader (Perkin Elmer, Waltham, MA). Results are reported as IC50, the concentration of compound needed to yield half maximal reduction in viability compared to vehicle-treated cells (control=100%). As shown in FIG. 11 and Table 11, h12F3 HGLF ADCs conjugated to vcMMAE, mp-dLAE-MMAE and mdpr-gluc-MMAE based linkers exhibit high cytotoxicity on 3D spheroids with similar potency as in 2D cultures.

TABLE 11
IC50 (ng/ml) values for h12F3 HGLF ADCs on tumor cell 3D spheroids

| Cell line | vc-MMAE(4) | dLAE-MMAE(4) | MDpr-PEG(12)-gluc-MMAE |
|---|---|---|---|
| RMUGS | 1.6 | 1.1 | 1.0 |
| NCI-N87 | 19.4 | 19.8 | 22.9 |

### Example 5: Antibody Internalization

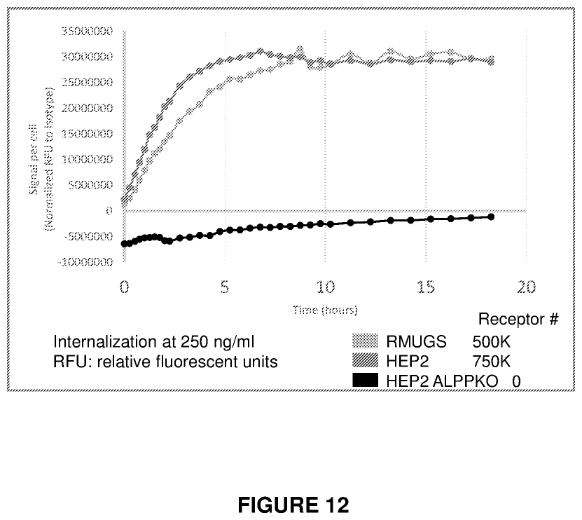

Internalization experiments were performed on RMUGS, Hep2 parental, and Hep2 ALPP knockout cell lines by automated fluorescence microscopy (IncuCyte S3, Essen Bioscience). Cells were seeded in 96-well flat clear bottom black tissue culture-treated microplates (Corning, Corning, NY) and left to adhere overnight at 37° C. h12F3 HGLF and non-targeting control antibody were labeled with IncuCyte FabFluor-pH Red Antibody Labeling Reagent (Essen Bioscience, Ann Arbor, MI) according to manufacturer's protocol. Volumes of test antibody, FabFluor reagent and medium required was calculated at 2× the final assay concentration and the FabFluor reagent was added at a molar ratio of 1:3 to the antibody. Antibody and FabFlour were mixed gently and incubated at 37° C. for 15 minutes, followed by addition of the antibody-FabFluor complex to each appropriate well of the cell containing plate. The final concentration of h12F3 HGLF and nonbinding control antibody per well was 250 ng/mL. The plates were arranged on microplate trays in the IncuCyte S3 (Essen Bioscience, Ann Arbor, MI) and scans were acquired using the Adherent Cell-by-Cell protocol. Phase data and red channel data (acquisition time set to 400 ms) were collected, with 4 images per well, at a minimum of every 0.5-2 hours for up to 20 hours with the objective set at 10×. Quantification of fluorescent signal intensity was performed using the IncuCyte software analysis tool. The analysis was refined and tuned per cell line utilizing a label-free cell count and manual image selection for preview and training of the algorithm. Upon completion of analysis, data was graphed using the IncuCyte software with graph metrics set to red mean intensity object average per cell normalized to nonbinding control. As shown in FIG. 12, h12F3 HGLF is internalized in ALPP expressing cells and the internalization is specific as ALPP knockout HEP2 cells do not internalize the naked antibody.

### Example 6: Kinetic Binding and pH Sensitivity

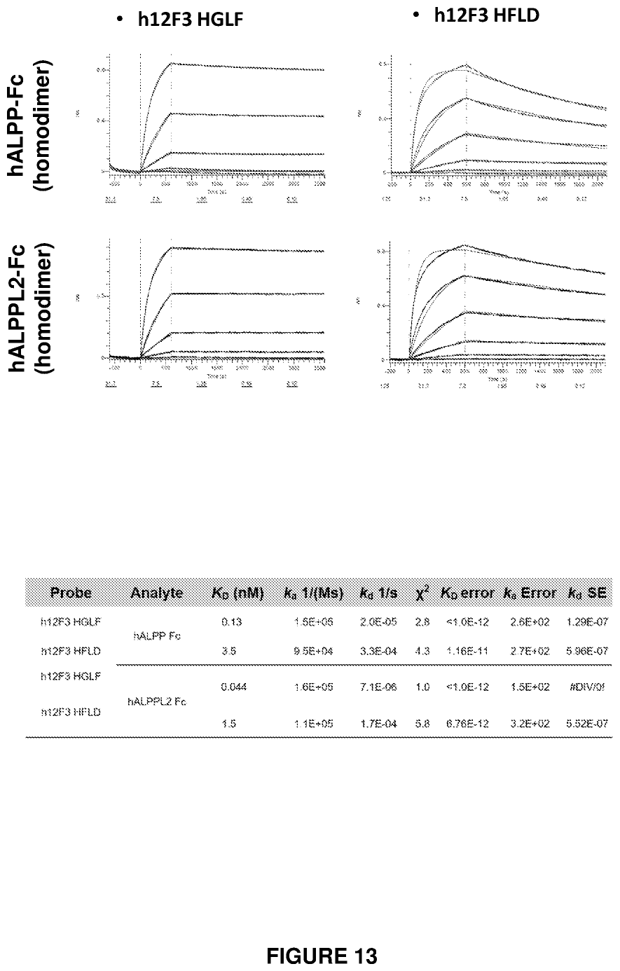

Bivalent affinity at pH 7.4, 37° C. was measured by Biolayer interferometry (BLI), on an Octet Red 384 system (ForteBio) with anti-human Fab-CH1 second generation (FAB2G) biosensors. Soluble human ALPP-Fc and ALPPL2-Fc fusion dimer proteins were generated in CHO cells to be used as the analyte. The antibodies, h12F3 HGLF and HFLD, were immobilized onto the biosensors at 3 μg/mL for 600 seconds before associating the titrated analytes, hALPP and hALPPL2, at 6 concentrations ranging from 0.12 to 125 nM for 600 seconds followed by a final 50 minute dissociation step in kinetic buffer (1% casein and 0.2% Tween20 in 1×PBS pH 7.4). After reference subtraction of the probe only curve, the data was globally fit with a 1:1 model with the Rmax sensor unlinked. With curve fitting of concentrations 31.3, 7.8, 1.95, 0.49, and 0.12 nM, bivalent binding of h12F3 HGLF to hALPP and hALPPL2 were measured to be 1.3E-10 M (k_{d} 2.0E-05 l/s/kₐ 1.5E5 l/Ms) and 4.4E-11 M (k_{d} 7.1E-06 l/s/kₐ 1.6E5 l/Ms), respectively. The HFLD variant had a 26.9 and 34-fold lower affinity for hALPP and hALPPL2, respectively, compared to HGLF as it is shown in FIG. 13).

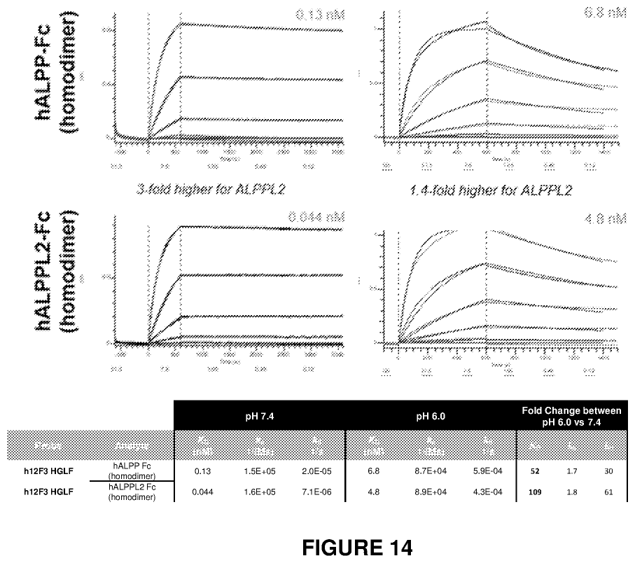

To evaluate pH sensitivity, the bivalent affinity at pH 6.0, 37° C. was determined using the same BLI method as the pH 7.4 experiment. The only difference was the use of a different kinetic buffer (1% casein and 0.2% Tween20 in phosphate citrate pH 6.0). Using 800 seconds of the dissociation with curve fitting of concentrations 125, 31.3, 7.8, 1.95, 0.49, and 0.12 nM, bivalent binding of h12F3 HGLF to hALPP and hALPPL2 were measured to be 6.8E-09 M (k_{d} 5.9E-04 l/s/kₐ 8.7E4 l/Ms) and 4.8E-9 M (k_{d} 4.3E-04 l/s/kₐ 8.9E4 l/Ms), respectively. As shown in FIG. 14, this was a 52-fold reduction for hALPP Fc and a 109-fold reduction for hALPPL2 from the pH 7.4 affinities.

### Example 7: In Vivo Antitumor Activity

NSG mice were subcutaneously inoculated with 5×10⁵ CAOV3p1, or 2.5×10⁶NCI-H1651 cells. Nude mice were subcutaneously inoculated with 1×10⁷ NCI-N87, 2×10⁶ RMUG-S, 1×10⁷ LoVo and 5×10⁶ HT-1376 cells. Each mouse was inoculated subcutaneously at the right flank in 0.1 ml of PBS with Matrigel (1:1) as specified by the manufacturer. Tumor growth was monitored with calipers and the mean tumor volume was calculated using the formula (0.5×[length×width²]). When the mean tumor volume reached approximately 100-200 mm³, mice were randomly separated in different cohorts including untreated conditions or dosed intraperitoneally with h12F3 HGLF or HFLD conjugated to mp-dLAE-MMAE or vcMMAE four times every four days (q4dx4) or three times every 7 days (q7dx3). Mice were euthanized when tumor volumes reached approximately 800-1000 mm³. The % TGI was defined as (1−(mean volume of treated tumors)/(mean volume of control tumors))×100%. All animal procedures were performed under a protocol approved by the Institutional Animal Care and Use Committee in a facility accredited by the Association for Assessment and Accreditation of Laboratory Animal Care.

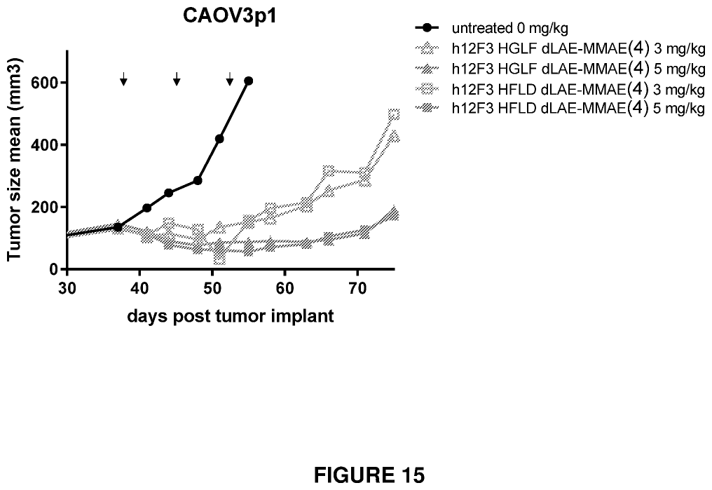

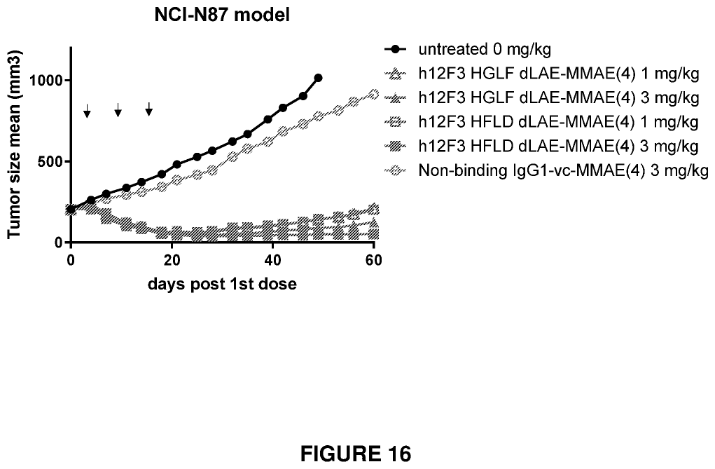

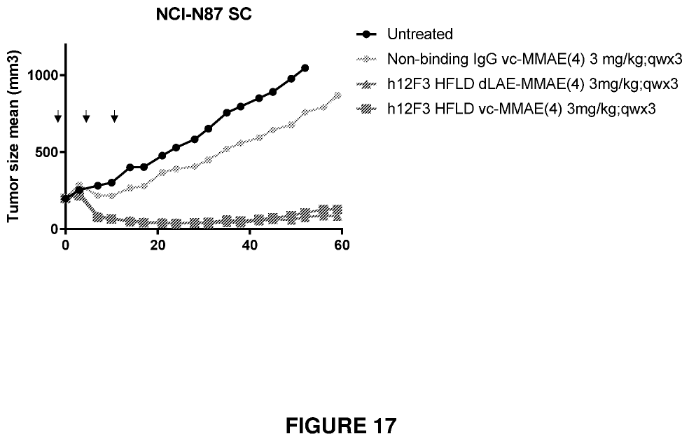

The resulting tumor volumes over time for untreated mice and mice treated with 3 and 5 mg/kg of h12F3 HGLF or HFLD-dLAE-MMAE are shown in FIG. 15 for an ovarian tumor model CAOV3. The resulting tumor volumes over time for untreated mice and mice treated with 1 and 3 mg/kg of h12F3 HGLF or HFLD-dLAE-MMAE are shown in FIG. 16 for a gastric tumor model NCI-N87. The resulting tumor volumes over time for untreated mice and mice treated with 3 mg/kg of h12F3 ADCs conjugated to vc-MMAE and dLAE-MMAE are shown in FIG. 17 for gastric tumor model NCI-N87. ALPP ADCs show similar antitumoral activity in vivo.

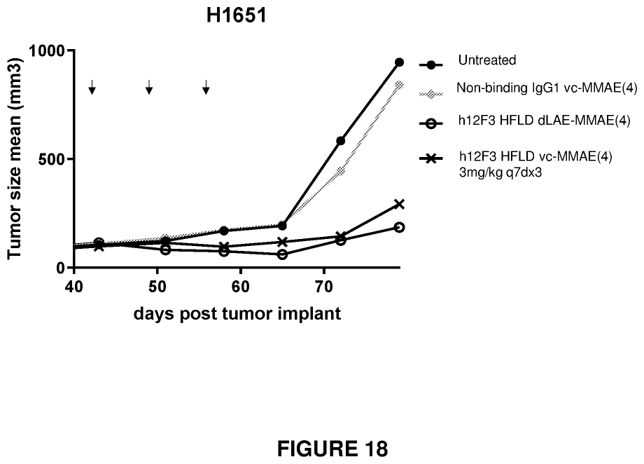

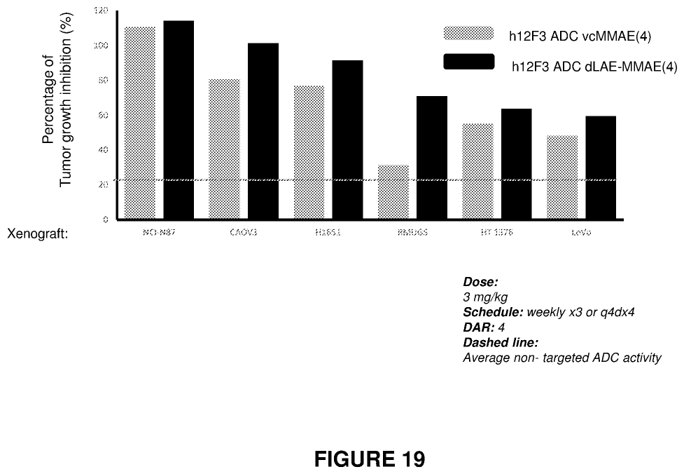

The resulting tumor volumes over time for untreated mice and mice treated with 3 mg/kg of h12F3 ADCs conjugated to vc-MMAE and dLAE-MMAE are shown in FIG. 18 lung tumor model NCI-H1651. ALPP ADCs show similar antitumoral activity in vivo. The resulting tumor growth inhibition across seven xenograft models is shown in FIG. 19. A bar graph summarizes the % tumor volume change in treatment arms relative to the control. The comparison was done at 3 mg/kg of h12F3-HFLD ADCs conjugated to vc-MMAE and dLAE-MMAE. Average antitumor activity of non-binding ADC control is shown by a dashed line.

In another set of assays, NSG mice were subcutaneously inoculated with 5×10⁵ CAOV3p1, NCG mice were subcutaneously inoculated with 5×106 SNU-2535, nude mice were subcutaneously inoculated with 1×10⁷ NCI-N87, and SCID mice were subcutaneously inoculated with 1×10⁷ HPAC. Each mouse was inoculated subcutaneously at the right flank in 0.1 ml of PBS with Matrigel (1:1) as specified by the manufacturer. Tumor growth was monitored with calipers and the mean tumor volume was calculated using the formula (0.5×[length×width²]). When the mean tumor volume reached approximately 100-200 mm³, mice were randomly separated in different cohorts including untreated conditions or dosed intraperitoneally with h12F3 HGLF conjugated to mp-dLAE-MMAE or me-vc-MMAE four times every 4 days (q4dx4) or three times every 7 days (q7dx3). Mice were euthanized when tumor volumes reached approximately 2-3000 mm³. The % TGI was defined as (1−(mean volume of treated tumors)/(mean volume of control tumors))×100%. All animal procedures were performed under a protocol approved by the Institutional Animal Care and Use Committee in a facility accredited by the Association for Assessment and Accreditation of Laboratory Animal Care.

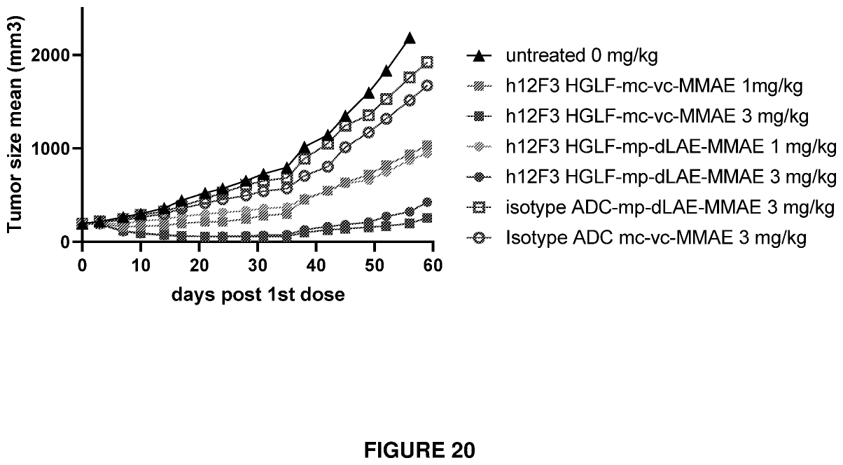

The resulting tumor volumes over time for untreated mice and mice treated with 1 or 3 mg/kg of h12F3 ADCs conjugated to vc-MMAE and dLAE-MMAE are shown in FIG. 20 for the gastric tumor model NCI-N87. ALPP ADCs show similar antitumoral activity in vivo.

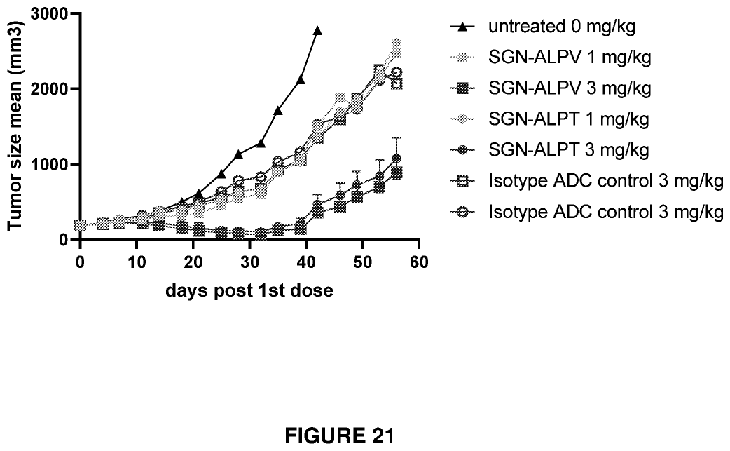

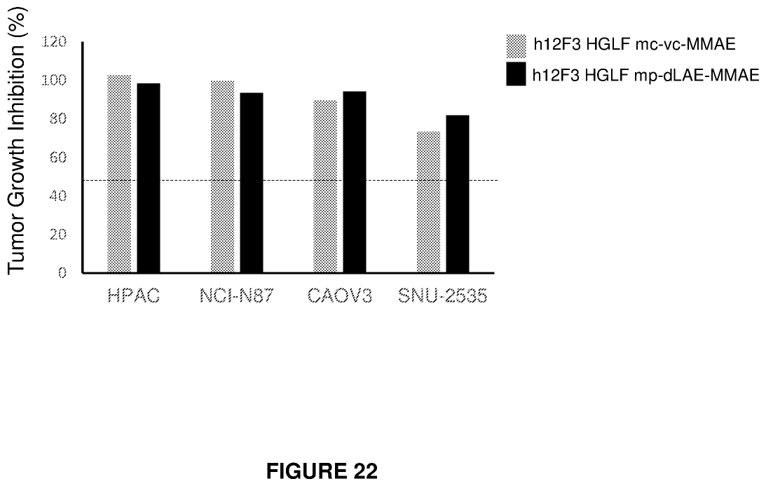

The resulting tumor volumes over time for untreated mice and mice treated with 1 or 3 mg/kg of h12F3 ADCs conjugated to me-vc-MMAE and mp-dLAE-MMAE are shown in FIG. 21 for the pancreatic tumor model HPAC. ALPP ADCs show similar antitumoral activity in vivo. The resulting tumor growth inhibition across four xenograft models is shown in FIG. 22. A bar graph summarizes the percentage tumor volume change in treatment arms relative to the control. The comparison was done at 3 mg/kg of h12F3-HGLF ADCs conjugated to vc-MMAE and dLAE-MMAE. Average antitumor activity of non-binding ADC control is shown by a dashed line.

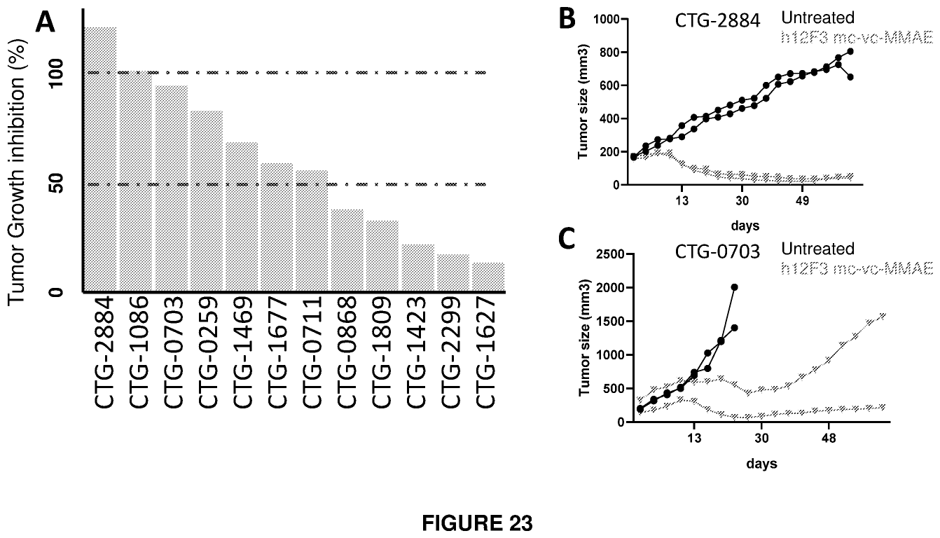

In another assay, antitumor activity on twelve patient-derived xenografts was performed in nude mice using a 2+2 experimental design. Briefly, when tumors of sufficient stock animals reached 1.0-1.5 cm³, tumors were harvested for re-implantation into pre-study animals. Pre-study animals were implanted unilaterally on the left flank with tumor fragments harvested from stock animals. Each animal was implanted from a specific passage lot and documented. When tumors reached an average tumor volume of 150-300 mm³, animals were matched by tumor volume into treatment or control groups to be used for dosing, and dosing initiated on Day 0. The h12F3-mc-vc-MMAE conjugate was dosed at 5 mg/kg (QW×3) and compared to a PBS-treated cohort. Tumor volumes were measured twice weekly. A final tumor volume measurement was taken on the day the study reached the endpoint. Beginning Day 0, tumor dimensions were measured twice weekly by digital caliper and data including individual and mean estimated tumor volumes (Mean TV±SEM) recorded for each group; tumor volume was calculated using the formula (1): TV=width2×length×0.52. At study completion, percent tumor growth inhibition (% TGI) values was calculated and reported for each treatment group (T) versus control (C) using initial (i) and final (f) tumor measurements by the formula (2): % TGI=1−(Tf−Ti)/(Cf−Ci). As shown in FIG. 23, the h12F3-me-vc-MMAE conjugate SGN-ALPV shows antitumor activity in 58% (7/12) of PDX models with heterogenous target expression. The responding models exhibited a TGI ranging from 55% to >100% at used doses. Antitumor activity was seen in PDX models from both chemotherapy-pretreated and naïve patients (FIG. 23, B and C).

### Example 8: Cross-Reactivity and Epitope Mapping

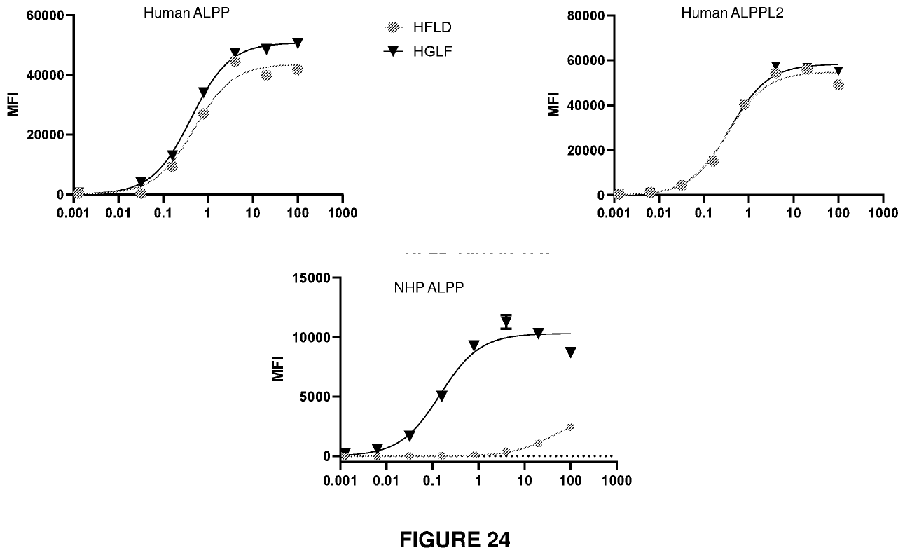

To confirm antibody cross-reactivity to ortholog ALPP protein, Macaca falsicularis ALPP gene (NHP ALPP) was transfected in HEK293 cells, and antibodies were screened by flow cytometry. Briefly, the KD for each resulting antibody was then determined by a saturation binding assay. 1×105 HEK293 cells stably expressing human ALPPL or ALPPL2 as well as NHP ALPP were aliquoted per well of a 96-well v-bottom plates. h12F3 HGLF and HFLD antibodies were added in concentrations ranging from 0.2 nM to 20 nM and incubated on ice for 60 minutes. Cells were pelleted and washed 3× with PBS/BSA followed by addition of 10 μg/ml of an APC labeled anti-human IgG goat secondary antibody and incubated on ice for an additional 60 minutes. Cells were pelleted and washed 3× with PBS/BSA and resuspended in 125 μL of PBS/BSA. Fluorescence was analyzed by flow cytometry, using percent of saturated fluorescent signal to determine percent bound and to subsequently calculate apparent KD. The apparent K_{D} for both antibodies are shown in FIG. 24. Importantly, while the antibody variant h12F3 HFLD exhibit a dramatic affinity reduction for the monkey ortholog gene, HGLF variant shows similar binding profile for both human and monkey placental alkaline phosphatases.

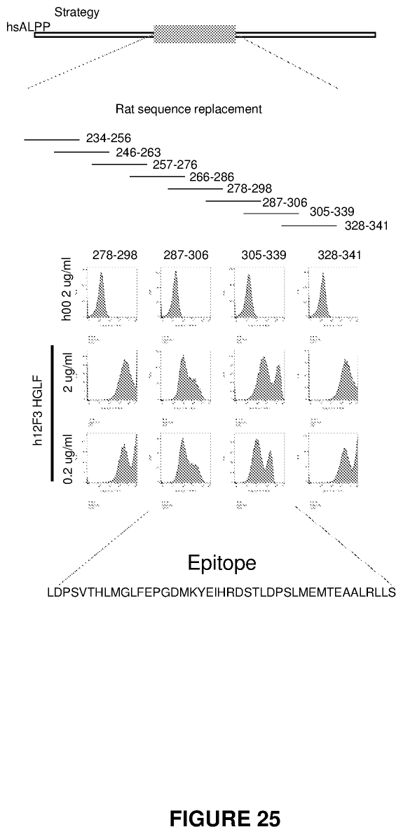

As the h12F3 HGLF does not cross react with the rat ALPP/ALPPL2 ortholog, human ALPP regions were exchanged with homologue regions from the rat ALPP. These constructs were transiently transfected in 2×10⁶ cells HEK293 cell using lipofectamine 3000 (1:1.5 DNA/lipofectamine ratio) according to the manufacturer's instructions. By using flow cytometry as described before, h12F3 HGLF binding epitope was evaluated on chimeric rat/human ALPP variant-expressing cells 48 h post-transfection. As shown in FIG. 25, h12F3 HGLF binding is impaired when the human ALPP regions containing the aa L287-S339 are replaced with rat ALPP sequences.

### Example 9: Antibody Kinetic Binding to Fc Receptors

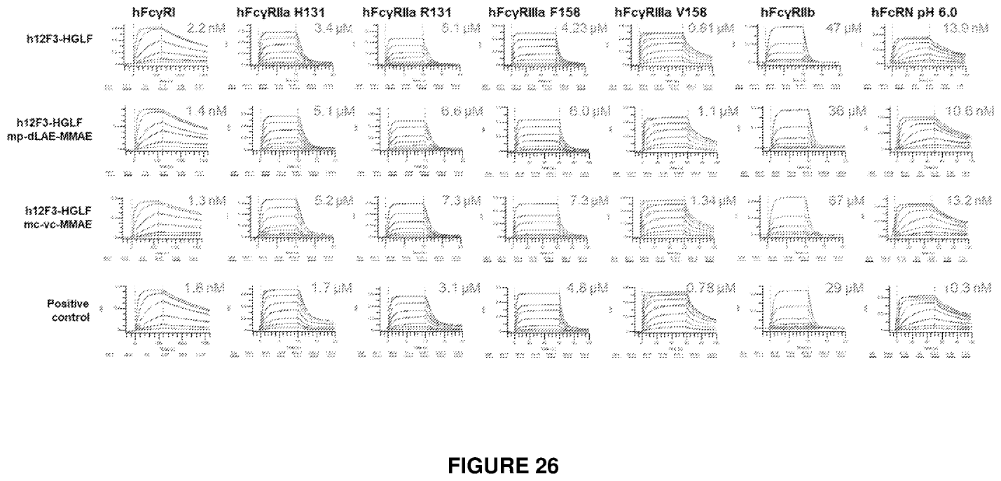

Antibody-based immune reactions are driven by interaction with Fc receptors on immune cells. Thus, to establish the ability of h12F3 HGLF, h12F3 HGLF-mc-vc-MMAE, or h12F3 HGLF-mp-dLAE-MMAE to interact with Fc receptors, binding kinetics with hFcγRI, hFcγRIIa H131, hFcγRIIa R131, hFcγRIIIa F158, hFcγRIIIa V158, and hscFcRN were assessed by Biolayer Interferometry (BLI). Biotinylated avi-tagged human Fc receptors fused with monomeric Fc (designed and expressed at Seagen) were loaded onto high precision streptavidin biosensors (from ForteBio) to responses around 0.4 nm for all receptors except for hFcγR1 with responses around 1.2 nm. An initial baseline was completed in immobilization buffer (0.1% BSA, 0.02% Tween 20, 1×PBS pH 7.4) followed by a second baseline in kinetic buffer (1% casein, 0.2% Tween 20, 1×PBS pH 7.4 for hFcγRI, IIa, IIIa, and IIb interactions and 1% BSA+0.2% Tween 20, Phosphate Citrate pH 6.0 for hscFcRN interactions). Titrated h12F3 HGLF, h12F3 HGLF-mc-vc-MMAE, h12F3 HGLF-mp-dLAE-MMAE and positive control mAb samples were associated and dissociated as follows: 600 s and 1000 s for hFcγRI, 10 s and 50 s for hFcγRIIa and hFcγRIIb, 60 s and 200 s for hFcγRIIIa, and 50 s and 200 s for hscFcRN in kinetic buffer, respectively. Sensorgrams were generated on an Octet HTX system (ForteBio) at 30° C. and globally fitted with the 1:1 kinetic Langmuir isotherm model (Rmax unlinked) after a reference subtraction of the antigen-loaded, 0 nM analyte sensor. Negative controls with the highest concentration of antibodies and ADCs (20 μM) with no Fc receptor immobilized were also included to verify the absence of nonspecific binding of the analyte to the streptavidin biosensors themselves. Specific loading concentrations and times of each receptor to the streptavidin sensors and concentrations of titrated analytes are listed (Table 12 and Table 13). In summary, parental antibody and me-vc-MMAE ADC binds all human Fc receptors as it is shown in FIG. 26. The highest affinity was for hFcγRI, ranging around 1.3-2.2 nM, while the second highest affinity was for hFcRN at approximately 10.6-13.9 nM. The affinities for hFcγRIIa and hFcγRIIIa variants ranged from 0.81 to 7.3 μM, and hFcγRIIb showed the weakest affinity ranging from 36 to 67 μM. Compared to the positive control mAb results, the affinities of h12F3 HGLF-mc-vc-MMAE and h12F3 HGLF-mp-dLAE-MMAE for all human Fc receptors were very similar and comparable to the parental antibody h12F3 HGLF.

TABLE 12
Immobilization concentrations and
times onto streptavidin biosensors

| | |
|---|---|
| hFcγI hmFc AAG A avi Biotin | (3.0 μg/mL, 400 s load) |
| hFcγRIIa H131 hmFc AAG avi Biotin | (0.7 μg/mL, 300 s load) |
| hFcγRIIa R131 hmFc AAG avi Biotin | (1.7 μg/mL, 300 s load) |
| hFcγRIIIa F158 hmFc AAG avi Biotin | (4.0 μg/mL, 300 s load) |
| hFcγRIIIa V158 hmFc AAG avi Biotin | (3.0 μg/mL, 300 s load) |
| hFcγRIIb hmFc AAG avi Biotin | (2.0 μg/mL, 300 s load) |
| hscFcRN hmFc IHH A avi Biotin | (7.0 μg/mL, 300 s load) |

TABLE 11
Concentrations of analytes

| | |
|---|---|
| h12F3¹ and Positive Control | 66.7, 22.2, 7.4, 2.47, |
| mAb with hFcγRI | 0.82, 0.27 nM |
| h12F3¹ and Positive Control | 20, 8.57, 3.67, 1.57, |
| mAb with hFcγRIIa, IIIa, and IIb | 0.67, 0.29, 0.12 μM |
| h12F3¹ and Positive Control | 500, 184.2, 67.9, 25, |
| mAb with hFcRN | 9.21, 3.39, 1.25 nM |

¹Same concentrations were used for 12F3 HGLF-based conjugates using mc-vc-MMAE and mp-dLAE-MMAE

### Antibody-Dependent Cellular Cytotoxicity (ADCC) by Primary NK Cells

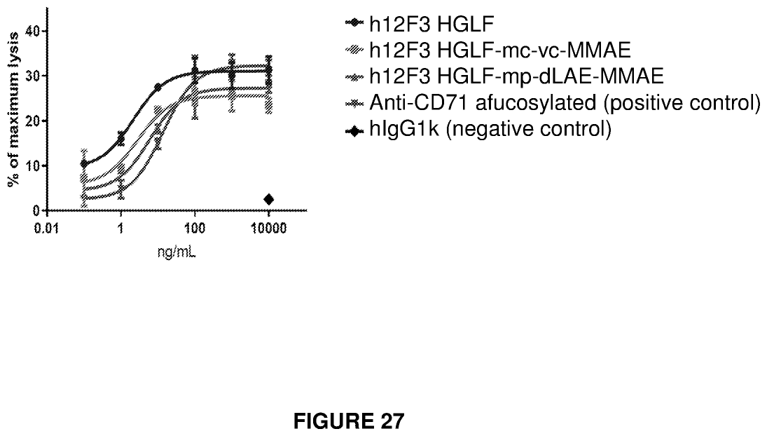

To establish whether the h12F3 HGLF backbone and derived conjugates elicits Antibody-Dependent Cellular Cytotoxicity (ADCC), ALPPL2-expressing cells were incubate with natural killer (NK) cells in the presence of h12F3 HGLF, h12F3 HGLF-me-vc-MMAE, or h12F3 HGLF-mp-dLAE-MMAE in vitro. After incubation, the percentage of cell lysis was measured. Briefly, effector cell preparation: On the day prior to the assay, peripheral blood mononuclear cells (PBMCs) were quickly thawed in a 37° C. water bath. Cells were transferred to 50-mL tubes containing AIM-V medium (Gibco, cat #12055091) supplemented with 5% heat-inactivated human serum (Gemini Bio-products, cat #100-512) (AIM-V/5% HIHS). Cells were centrifuged at 1500 rpm for 10 minutes. PBMCs were resuspended in AIM-V/5% HIHS containing DNase I (Sigma-Aldrich, cat #D5025) at a final concentration of 20 μg/mL (make a 1:50 dilution out of 1 mg/mL stock solution) and incubated at 37° C. for 10 to 15 minutes. Cells were pelleted by centrifugation as above and resuspended in AIM-V/5% HIHS. Cells were counted and seeded in T150 flasks at a concentration of 2-4×108 cells/flask in 25 mL per flask. Cells were incubated undisturbed overnight in a 37° C., 5% CO2, humidified incubator. The following day, the non-adherent cells were collected, and flasks were vigorously rinsed 3 times (7 mL) with PBS. Rinses were combined with the non-adherent cells and pelleted by centrifugation at 1500 rpm for 7 minutes. Cells were resuspended in a small volume to count (2 mL), and cell suspension was adjusted to a concentration of 5×107 cells/mL in PBS+2% FBS (as recommended by the EasySep protocol). NK cells were isolated by negative selection as per the EasySep Human NK cell enrichment kit (Stem Cell Technologies, cat #19055) instructions. Enriched effector cells were then suspended in RPMI/1% FBS at a concentration of 7.2×105 cells/mL (such that 70 μL contains ˜5×104 effector cells). Target cell preparation: ALPPL2-expressing LoVo cells were collected and counted. Next, 5×106 cells were removed and pelleted by centrifugation. Cells were resuspended in 100 μL of FBS. Then, 100 μL (approximately 100 μCi) of Cr-51 (Perkin Elmer Health Sciences, Inc., cat #NEZ030S) was added to cells and mixed by tapping gently. Cells were placed in a 37° C., 5% CO2, humidified incubator to label for 1 hour, and the tube was tapped occasionally to suspend the cells. Cells were washed 3 times with RPMI/1% FBS. Tube was tapped to loosen the cell pellet between washes. After washes, cells were resuspended in 10 mL RPMI/1% FBS and counted. Then, 7.2×105 cells were removed and suspended in a total volume of 10 mL assay medium such that 70 μL is equivalent to ˜5×103 target cells. Preparation of ADC and antibody dilutions and plate assembly: Antibodies and ADC were diluted in assay medium at 3× concentration. Antibodies tested were h12F3 HGLF, h12F3 HGLF-mc-vc-MMAE, or h12F3 HGLF-mp-dLAE-MMAE, CD71-binding afucosylated antibody, and isotype control. Antibodies were added to assay plate just prior to addition of Cr-labeled target cells. In addition, 70 μL and 140 μL assay medium was added in place of antibody to control wells representing total and spontaneous release controls, respectively. Finally, target cells were mixed, and 70 μL was added to each test and control well of the 96-well plate. Targets were incubated with mAbs in a 37° C., 5% CO2, humidified incubator for 30 minutes. Then, 70 μL (5×104) of effector cells were added to each test well, while 70 μL 3% Triton X-100 was added to total release wells and mixed. The plate was returned to the 37° C., 5% CO2, humidified incubator for 4 hours. After incubation, 35 μL supernatant was transferred to Luma plate(s). Luma plate(s) were dried overnight, then covered with sealing tape and read on a Perkin Elmer TopCount NXT Microplate Scintillation Counter. Analysis was done by calculating % specific lysis as follows (analyzed with GraphPad Prism): % specific lysis=[(test cpm−background cpm)÷(total cpm−background cpm)]×100. As it is shown in FIG. 27, in the presence of h12F3 HGLF antibody as well as the h12F3 HGLF-mc-vc-MMAE, and h12F3 HGLF-mp-dLAE-MMAE mediates NK cells cellular cytotoxicity in vitro. This activity is similar to the positive control and mediated by the presence of the target on cells as non-binding antibody failed to stimulate the effector cells.

### Antibody-Dependent Cellular Cytotoxicity

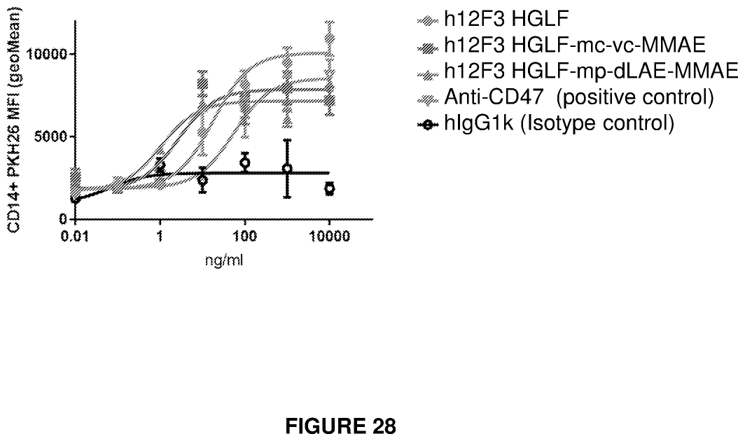

To determine whether h12F3 HGLF, h12F3 HGLF-mc-vc-MMAE, or h12F3 HGLF-mp-dLAE-MMAE exhibits ADCP activity, antibody- or ADC-coated fluorescent cells expressing ALPP/ALPPL2 were co-incubated with primary macrophages, and phagocytosis was measured with fluorescent flow cytometry. Briefly, LoVo tumor cells were fluorescently labeled with PKH26 according to the manufacturer's instructions. Cells were harvested off the culture dish with 0.05% Trypsin EDTA and washed once in 1×PBS. Cells were resuspended in 1 mL Diluent C included in the PKH26 Red Fluorescent Cell Membrane Labeling Kit (Sigma-Aldrich, cat #PKH26GL-1KT). In a separate tube, 1 mL Diluent C+4 μL PKH26 dye was added and pipetted up and down to mix. Staining solution was transferred to resuspended cells and quickly mixed by pipetting up and down several times. Cells were incubated at room temperature for 5 minutes, and the labeling reaction was stopped by adding 2 mL FBS. Cells were washed 3× with RPMI/10% FBS and resuspended in PBS at a concentration of 0.8×106 cells/mL. Labeled target cells were transferred to a 96-well U-bottom plate and treated with test antibody, ADC or isotype control antibody using the following steps. In a separate 96-well U-bottom plate, 10× stocks of mAb, ADC and isotype control in PBS were serially diluted 1:10 in PBS, and 33 μL/well was added to appropriate wells of cells in the U-bottom plate. Plate was incubated at room temperature for 30 minutes, centrifuged, and washed once with 200 μL/well medium (RPMI/10% FBS). Cells were resuspended in 330 μL/well medium (RPMI/10% FBS). The day before the assay, PBMCs from 2 healthy donors were thawed at 37° C. in a water bath and transferred into RPMI/10% FBS (0.1-0.2 EU/mL). A total of 0.7×106 PBMCs per well were added to 48-well flat-bottom plates and allowed to adhere overnight. Old medium (and non-adherent cells) was aspirated and replaced with 200 μL fresh medium. Next, 100 μL of labeled, treated target cells from each well was transferred into corresponding wells of adhered monocyte/macrophage flat-bottom plate in triplicate, and the plate was incubated at 37° C. overnight for 16-18 hours. All cells in the 48-well plate were harvested by collecting supes, collecting wash with 1×PBS, and detachment with 1× Versene. Macrophages were fluorescently labeled using the following steps: target cells and macrophages were collected in U-bottom plates, centrifuged, resuspended in 50 μL FACS staining buffer containing human Fc fragment blocking agent (1:20 dilution), and incubated on ice for 30 minutes. Next, 50 μL of a 1:50 dilution of CD14-BV421 and CD45-APC-Cy7 antibodies diluted in FACS staining buffer was added into each well and incubated on ice in foil for 30 minutes. Cells were centrifuged, washed 2× with FACS buffer, and resuspended in 1×PBS for subsequent flow cytometry analysis on an Attune NxT Flow Cytometer. YL1 GeoMean Fluorescence of CD14+/CD45 cells (MFI of CD14+/CD45+ cells) was analyzed using FlowJo, and then values were exported to Excel and into GraphPad Prism for further data analysis. Phagocytosis is reported as MFI of CD14+ cells. As it is shown in FIG. 28, the presence of h12F3 HGLF, h12F3 HGLF-mc-vc-MMAE, or h12F3 HGLF-mp-dLAE-MMAE conjugates enables phagocytosis of target expressing cells with a similar kinetics than the positive control (anti-CD47 antibody). This activity is dependent on the presence of the ALPPL2 expression on target cells as the non-binding antibody does not elicit any cell death.

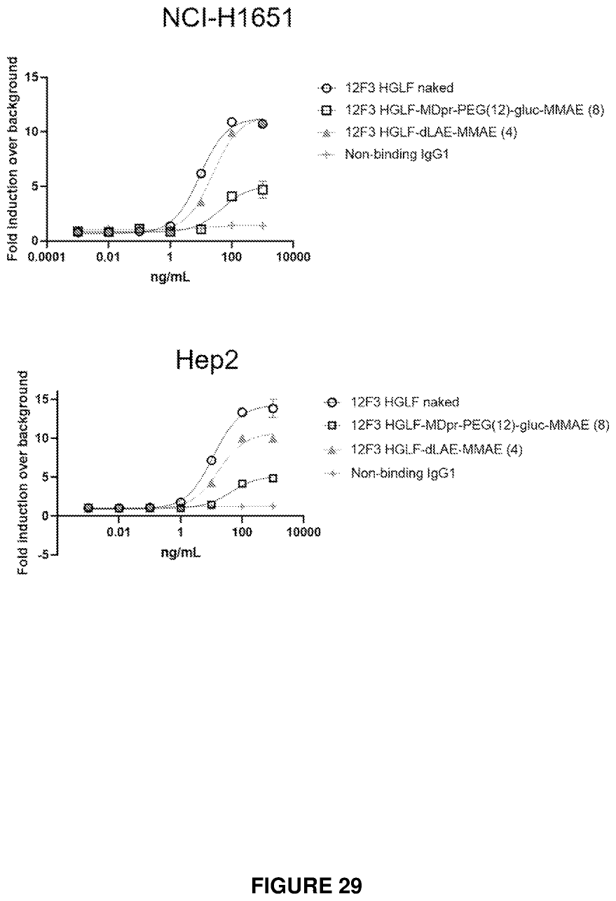

In another assay, FcgRIII-dependent Antibody Dependent Cellular Cytotoxicity (ADCC) was measured by using a surrogate luciferase-based mediated bioassay from Promega. Briefly, ALPP/ALPPL2-expressing cells were plated on 96-well plates and co-cultured with ADCC Bioassay Effector Cells (Promega) in the presence of increasing amount of naked h12F3 antibody or h12F3 HGLF antibody conjugated to 4 or 8 molecules of me-vc-PABC-MMAE or MDpr-PEG(12)-gluc-MMAE, respectively. 24 h post-treatment, cells were incubated with Bio-Glo (Promega) according to the manufacturing and luminescence was measured with an Envision platform. As shown in FIG. 29, h12F3 HGLF is capable of activating FcgRIII signaling in reporter cell line in a similar kinetics that the h12F3 HGLF ADC conjugated mc-vc-PABC-MMAE. Conjugation to 8 molecules of MDpr-PEG(12)-gluc-MMAE has reduced ADCC activity compared to naked h12F3 HGLF.

### Example 10: Immunogenic Cell Death

### Signal Pathway Activation for Immunogenic Cell Death

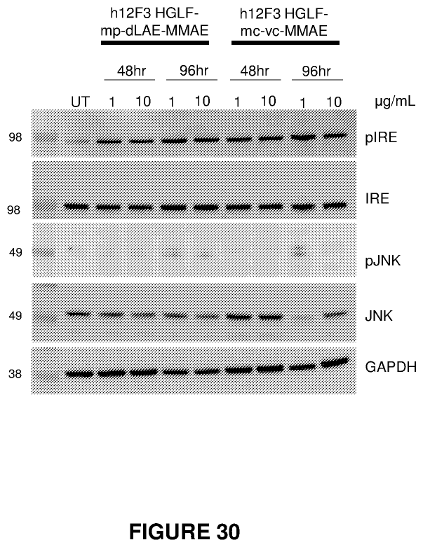

To determine whether h12F3 HGLF-mc-vc-MMAE and h12F3 HGLF-mp-dLAE-MMAE can activate hallmarks of ICD, ALPP/ALPPL2 expressing cells were treated with ADCs and immunoblotting was used to establish the phosphorylation status of IRE an JNK pathways. Briefly, four million LOVO cells were plated in 10 cm TC treated dishes in 10 mL compete growth media (Ham's F-12K (Kaighn's) media+10% FBS) and allowed to adhere overnight. Cells were treated with 10 nM MMAE or 1 and 10 μg/mL of h12F3 HGLF-me-vc-MMAE or h12F3 HGLF-mp-dLAE-MMAE in complete media. Cells were then incubated in their treated media for 48 or 96 hours. Cells were collected, washed and resuspended in 500 uL cold PBS and transferred to Eppendorf tubes. Samples were spun at 300×g for 3 min at 4 degrees. Supernatants were removed and cells were resuspended in RIPA lysis buffer containing protease and phosphatase. After incubation for 5 min on ice, samples were spun at 17000×g for 10 min at 4 degrees and the supernatants were collected, and stored at −80. Quantified samples were resolved in a NuPAGE 4-12% bis-tris gels and run in either MES running buffer for smaller proteins or MOPS for larger proteins (165 v for 40 minutes). Gels were transferred to PVDF membranes using the iBlot2 (20 v for 7 min). Membranes were briefly rinsed in DI water and then placed in blocking buffer (TBS+0.10% tween-20+5% BSA) overnight at 4 degrees. Blots were then incubated with primary antibodies against IRE, JNK, p-IRE, or p-JNK at 1:1000 dilutions in blocking buffer for 2 hours at room temperature. p-ERK was used at 1:500 and incubated in the same manner. Blots were washed 3× with TBST (TBS+0.1% Tween-20). Anti-Rabbit peroxidase secondary antibody was prepared at 1:10,000 dilution in blocking buffer. Blots were incubated with secondary antibody for 1 hour at room temperature. Blots were again washed 3× with TBST. Blots were developed using SignalFire ECL and imaged on an Amersham Imager 600. Blots were then stripped and re-probed for GAPDH as a loading control and blotted as described above. As it is shown in FIG. 30, incubation of LoVo cells with either h12F3 HGLF-mc-vc-MMAE or h12F3 HGLF-mp-dLAE-MMAE increase the phosphorylation levels of pIRE and pJNK, which play key roles in the activation of immunogenic cell death process.

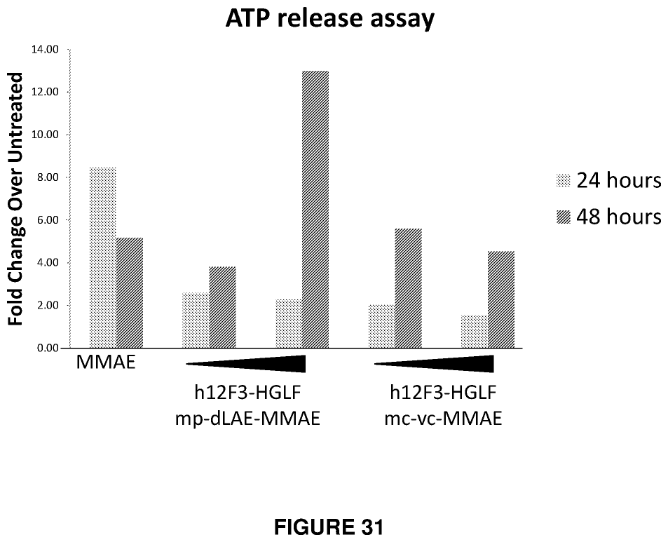

To establish whether treatment with h12F3 HGLF ADC leads to the ATP release in the culture media, 600,000 LOVO cells were plated in 6 well TC treated dishes in 2 mL compete growth media (Ham's F-12K (Kaighn's) media+10% FBS) and allowed to adhere overnight. Solutions of 10 nM MMAE, 1 or 10 μg/mL of h12F3 HGLF mp-dLAE-MMAE or mc-vc-MMAE were prepared in complete media. Cells were then incubated in their treated media for 24, 48, or 72 hours. At each endpoint, 500 uL supernatant were collected from each well (sample). Supernatants were spun at 200×g for 1 min at 4 degrees to carefully remove any cell debris. 3 50 uL aliquots (for triplicate data) of each supernatant were then placed into a black-walled, clear bottom 96 well plate. 50 uL of reconstituted Cell Titer Glo were then added to all wells containing supernatants. The plate was covered and protected from light. The plate was then read on an Envision plate reader. Raw luminescence data from the triplicate data were averaged for all samples. To determine the fold change compared to untreated samples, the average luminescence for the experimental samples were divided by the average for the untreated samples. As shown in FIG. 31, both h12F3 HGLF mp-dLAE-MMAE or me-vc-MMAE conjugates led to the release of ATP a hallmark of immunogenic cell death.

### Example 11: Pharmacokinetics

Pharmacokinetic evaluation of humanized h12F3 ADCs was performed in non-human primates. Antibody drug conjugates including h12F3 HGLF-vc-MMAE(4) and HGLF-dLAE-MMAE(4) were dosed once at 1 mg/kg and plasma samples were collected at specified time points. Total h12F3 HGLF-vc-MMAE(4) and HGLF-dLAE-MMAE(4) cynomolgus monkey plasma levels were analyzed using Gyrolab (Gyros Protein Technologies, Sweden) 1-Step Generic Total Antibody (gTAb) assay. In short, assay standards and quality control samples (QCs) were prepared with the dosed test article diluted in pooled cynomolgus monkey K2EDTA plasma (BioIVT). Study samples with test article concentrations outside the quantitative limits of the assay were diluted into range with drug naïve cynomolgus monkey K2EDTA plasma. Standards, QCs, and study samples were diluted to a minimum required dilution (MRD) of 1:20 into Rexxip HX buffer (Gyros Protein Technologies, Sweden). A 30 nM equimolar master mix solution was prepared by diluting biotinylated anti-human kappa light chain (Seagen) and AlexaFlour-647 anti-human Fcγ (Jackson Immunoresearch) in 1× phosphate buffered saline solution containing 0.01% (v/v) tween-20 (PBST). An equal volume of MRD standards, QCs, or study samples and master mix solution were mixed. The resulting solution was incubated protected from light, with shaking for 1-2 hours at room temperature. After the incubation, the solution was transferred to a 96-well PCR plate and added to a Gyrolab Bioaffy 1000 CD (Gyros Protein Technologies, Sweden) in which sample is passed over a streptavidin affinity column within the CD. Columns were washed 4× with PBST and the associated fluorescence was interrogated on the column at 635 nm. The fluorescent response of the calibrators was fitted to a 5-parameter logistic regression (5-PL) using Gyrolab Evaluator software. Total h12F3 HGLF-vcMMAE and HGLF-dLAE-MMAE QC and study sample concentrations were interpolated from the respective fitted standard curve and used for pharmacokinetic evaluation. The PK parameters were determined using Phoenix WinNonlin (version 8.2, Certara USA, Inc.) by non-compartmental analysis (NCA), as appropriate. The following PK parameters were determined: area under the plasma concentration-time curve to 21 days (AUC0-21), maximum observed plasma concentration (Cmax), terminal half-life, clearance (Cl), and calculated volume of distribution at steady-state (Vss). AUC was calculated using the linear trapezoidal linear method. Half-lives, Cl and Vss were reported for plasma concentration-time profiles that had an adjusted R2 of ≥0.8 and extrapolated AUC0-inf<20%. Resulting pharmacokinetic parameters are found in Table 14, showing that h12F3 HGLF conjugates with both vcMMAE and mp-dLAE-MMAE display similar antibody conjugated MMAE pharmacokinetic profiles, with no evidence of target mediated drug disposition when compared to a non-binding ADC control.

TABLE 14

| | | AUC_{0-21 d}/Dose | Cmax/Dose | Half-life |
|---|---|---|---|---|
| Antibody | payload | (day*kg*μg/mL/mg) | (kg*μg/mL/mg) | (day) |
| h12F3 | vc-MMAE(4) | 97.81 | 30.27 | 9.11 |
| HGLF | dLAE-MMAE(4) | 109.87 | 29.86 | NR |
| Non-binding | vc-MMAE(4) | 91.88 | 24.14 | 8.00 |
| IgG1 | dLAE-MMAE(4) | 91.33 | 31.18 | 6.83 |

For quantitation of antibody conjugated MMAE (acMMAE), plasma samples first went through an immunocapture to isolate the ADC (MAbSelect, GE Healthcare) for one hour at 2-8° C. The bound samples were washed using the papain digestion buffer (20 mM KPO4.10 mM EDTA, 20 mM Cysteine HCl) and then 2 mg/mL papain in the digestion buffer was added to each sample. Samples were incubated at 37 C for four hours to enzymatically release acMMAE. The resulting released acMMAE was extracted using solid phase extraction. Each sample was then analyzed using normal-phased UPLC (Betasil, ThermoFisher) coupled with tandem mass spectrometry (Sciex 6500+ Triple Quad). Table 15 shows similar pharmacokinetic parameters for antibody conjugated MMAE using h12F3 HGLF vc-MMAE and HGLF-dLAE-MMAE conjugates, with the latter showing an extended half-life.

TABLE 15

| | | AUC_{0-21 d}/Dose | Cmax/Dose | Half-life |
|---|---|---|---|---|
| Antibody | payload | (day*kg*μg/mL/mg) | (kg*μg/mL/mg) | (day) |
| h12F3 | vc-MMAE(4) | 49.19 | 30.67 | 4.47 |
| HGLF | dLAE-MMAE(4) | 47.97 | 29.50 | 8.39 |

The tolerability of h12F3 HGLF ADCs was evaluated in cynomolgus macaques as a pharmacologically relevant species with comparable binding affinity to human and cynomolgus ALPP orthologs. Female monkeys were administered either h12F3 HGLF-vc-MMAE(4) at 5 mg/kg or h12F3 HGLF-dLAE-MMAE(4) at 5, 8, 9, and 10 mg/kg, respectively, weekly for four weeks (q1w×4). Toxicologic assessments included body weights, clinical observations, hematology, coagulation, serum chemistry, and TK. At terminal (1 week post last dose) and recovery (4 weeks post last dose) necropsy, gross pathology examination was performed, and tissues were examined histopathologically. The maximum tolerated dose of h12F3 HGLF-vc-MMAE(4) was 5 mg/kg and h12F3 HGLF-dLAE-MMAE(4) was 9 mg/kg (Table 16). Bone marrow toxicity consistent with the pharmacology of MMAE was detected for both ADCs via hematology and anatomic pathology assessments and considered the dose limiting toxicity. Additional toxicities of alveolar macrophage accumulation in the lung, decreased number of secondary and tertiary follicles in the ovaries, and lymphoid depletion in the thymus

TABLE 16

| | MTD | Dose Limiting |
|---|---|---|
| Test Article | (mg/kg) | Toxicity |
| h12F3 HGLF-mp- | 9 (q1wx4) | Bone Marrow |
| dLAE-MMAE (4) | | |
| h12F3 HGLF-mc- | 5 (q1wx4) | Bone Marrow |
| vc-MMAE (4) | | |

### INCORPORATION BY REFERENCE

All references cited herein, including patents, patent applications, scientific papers, textbooks and the like are incorporated by reference in their entirety.

1. An antigen binding protein or fragment thereof that binds ALPP and/or ALPPL2, the antigen binding protein or fragment thereof comprising the following 6 CDRs:
a CDR-H1 comprising the amino acid sequence of SEQ ID NO: 56 or SEQ ID NO: 60;
a CDR-H2 comprising the amino acid sequence of SEQ ID NO: 57 or SEQ ID NO: 61;
a CDR-H3 comprising the amino acid sequence of SEQ ID NO: 58 or SEQ ID NO: 62;
a CDR-L1 comprising the amino acid sequence of SEQ ID NO: 63 or SEQ ID NO: 68;
a CDR-L2 comprising the amino acid sequence of SEQ ID NO: 64 or SEQ ID NO: 69; and
a CDR-L3 comprising the amino acid sequence of SEQ ID NO: 65 or SEQ ID NO: 70;
wherein the CDRs are determined by Kabat or IMGT.

2. The antigen binding protein or fragment of claim 1 comprising a CDR-H1 comprising the amino acid sequence of SEQ ID NO: 56; a CDR-H2 comprising the amino acid sequence of SEQ ID NO: 57; a CDR-H3 comprising the amino acid sequence of SEQ ID NO: 58; a CDR-L1 comprising the amino acid sequence of SEQ ID NO: 63, a CDR-L2 comprising the amino acid sequence of SEQ ID NO: 64, and a CDR-L3 comprising the amino acid sequence of SEQ ID NO: 65; wherein the CDRs are determined by Kabat.

3. The antigen binding protein or fragment of claim 1 comprising a CDR-H1 comprising the amino acid sequence of SEQ ID NO: 60; a CDR-H2 comprising the amino acid sequence of SEQ ID NO: 61; a CDR-H3 comprising the amino acid sequence of SEQ ID NO: 62; a CDR-L1 comprising the amino acid sequence of SEQ ID NO: 68, a CDR-L2 comprising the amino acid sequence of SEQ ID NO: 69, and a CDR-L3 comprising the amino acid sequence of SEQ ID NO: 70; wherein the CDRs are determined by IMGT.

4. The antigen binding protein or fragment of claim 1 that comprises a VH and a VL, wherein the VH has at least 80%, 85%, 90%, 95% or 99% amino acid sequence identity to the amino acid sequence of SEQ ID NO: 15, and wherein the VL has at least 80%, 85%, 90%, 95% or 99% amino acid sequence identity to the amino acid sequence of SEQ ID NO: 30.

5. The antigen binding protein or fragment of claim 1 that comprises a VH and a VL, wherein the VH comprises the amino acid sequence of SEQ ID NO: 15.

6. The antigen binding protein or fragment of claim 1 that comprises a VH and a VL, wherein the VL comprises the amino acid sequence of SEQ ID NO: 30.

7. The antigen binding protein or fragment of claim 1 that comprises a VH and a VL, wherein the VH comprises the amino acid sequence of SEQ ID NO: 15, and the VL comprises the amino acid sequence of SEQ ID NO: 30.

8. The antigen binding protein or fragment of claim 1, comprising a heavy chain (HC) comprising the amino acid sequence of SEQ ID NO: 40.

9. The antigen binding protein or fragment of claim 1, comprising a light chain (LC) comprising the amino acid sequence of SEQ ID NO: 50.

10. The antigen binding protein or fragment of claim 1, comprising a HC comprising the amino acid sequence of SEQ ID NO: 40 and comprising an LC comprising the amino acid sequence of SEQ ID NO: 50.

11. The antigen binding protein or fragment of claim 1, wherein the antigen binding protein is a monoclonal antibody or fragment thereof.

12. The antigen binding protein or fragment of claim 1, wherein the antigen binding protein is a humanized antibody or fragment thereof.

13. The antigen binding protein or fragment of claim 1, wherein the fragment is selected from a Fab, Fab′, Fv, scFv or (Fab′)₂ fragment.

14. An antibody-drug conjugate comprising the antibody or antigen-binding fragment of claim 1 conjugated to a cytotoxic or cytostatic agent.

15. The antibody-drug conjugate of claim 14, wherein the antibody or antigen-binding fragment is conjugated to the cytotoxic or cytostatic agent via a linker.

16. The antibody-drug conjugate of claim 14, wherein the cytotoxic or cytostatic agent is a monomethyl auristatin.

17. The antibody-drug conjugate of claim 14, wherein the monomethyl auristatin is monomethyl auristatin E (MMAE).

18. The antibody-drug conjugate of claim 17, wherein the antibody or antigen binding fragment thereof is conjugated to MMAE via an enzyme-cleavable linker unit.

19. The antibody-drug conjugate of claim 18, wherein the enzyme-cleavable linker unit comprises a Val-Cit linker.

20. The antibody-drug conjugate of claim 19, wherein the antibody or antigen binding fragment thereof is conjugated to MMAE via a linker unit that has the formula: -Aₐ-W_{w}-Y_{y}-; wherein -A- is a stretcher unit, a is 0 or 1; -W- is an amino acid unit, w is an integer ranging from 0 to 12; and -Y- is a spacer unit, y is 0, 1, or 2.

21. The antibody-drug conjugate of claim 20, wherein the stretcher unit has the structure of Formula I below; wherein the amino acid unit is Val-Cit; and wherein the spacer unit is a p-aminobenzyl alcohol (PABC) group having the structure of Formula II below;

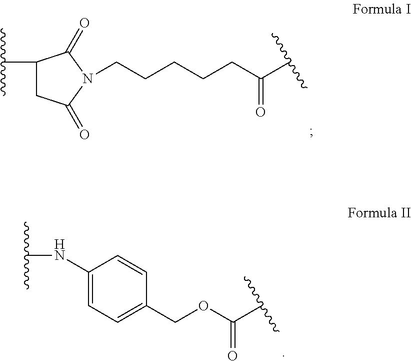 [SMILES: *C(=O)CCCCCN1C(=O)CC(*)C1=O ; *Nc1ccc(COC(*)=O)cc1]

22. The antibody-drug conjugate of claim 14, wherein the linker is attached to monomethyl auristatin E forming an antibody-drug conjugate having the structure: 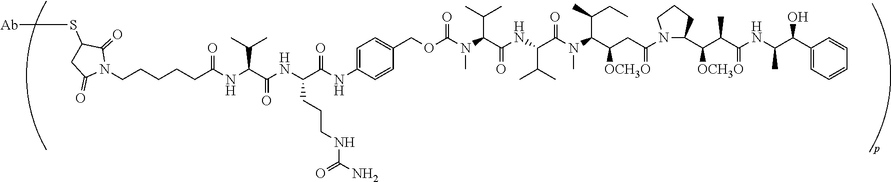
wherein Ab is the antibody h12F3 and p denotes a number from 1 to 16.

23. The antibody-drug conjugate of claim 22, wherein the average value of p in a population of the antibody-drug conjugate is about 4.

24. The antibody-drug conjugate of claim 14, wherein the antibody-drug conjugate is represented by the structure: 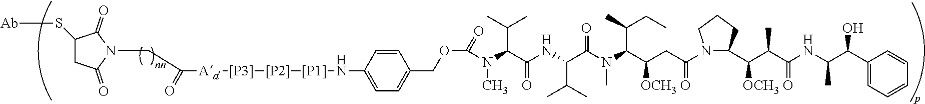
or a pharmaceutically acceptable salt thereof, wherein:
Ab is the antibody h12F3 and p denotes a number from 1 to 12;
subscript nn is a number from 1 to 5;
subscript a′ is 0, and A′ is absent;
P1, P2, and P3 are each an amino acid, wherein:
a first one of the amino acids P1, P2, or P3 is negatively charged;
a second one of the amino acids P1, P2, or P3 has an aliphatic side chain with hydrophobicity no greater than that of leucine; and
a third one of the amino acids P1, P2, or P3 has hydrophobicity lower than that of leucine,
wherein the first one of the amino acids P1, P2, or P3 corresponds to any one of P1, P2, or P3, the second one of the amino acids P1, P2, or P3 corresponds to one of the two remaining amino acids P1, P2, or P3, and the third one of the amino acids P1, P2, or P3 corresponds to the last remaining amino acids P1, P2, or P3,
provided that -P3-P2-P1- is not -Glu-Val-Cit- or -Asp-Val-Cit-.

25. The antibody-drug conjugate of claim 24, wherein subscript nn is 2.

26. The antibody-drug conjugate of claim 24, wherein:
the P3 amino acid of the tripeptide is in the D-amino acid configuration;
one of the P2 and P1 amino acids has an aliphatic side chain with hydrophobicity lower than that of leucine; and
the other of the P2 and P1 amino acids is negatively charged.

27. The antibody-drug conjugate of claim 24, wherein the P3 amino acid is D-Leu or D-Ala.

28. The antibody-drug conjugate of claim 24, wherein the P3 amino acid is D-Leu or D-Ala, the P2 amino acid is Ala, Glu, or Asp, and the P1 amino acid is Ala, Glu, or Asp.

29. The antibody-drug conjugate of claim 24, wherein -P3-P2-P1- is -D-Leu-Ala-Asp-, -D-Leu-Ala-Glu-, -D-Ala-Ala-Asp-, or -D-Ala-Ala-Glu-.

30. The antibody-drug conjugate of claim 24, wherein -P3-P2-P1- is -D-Leu-Ala-Glu-.

31. The antibody-drug conjugate of claim 24, wherein the antibody-drug conjugate is represented by the structure: 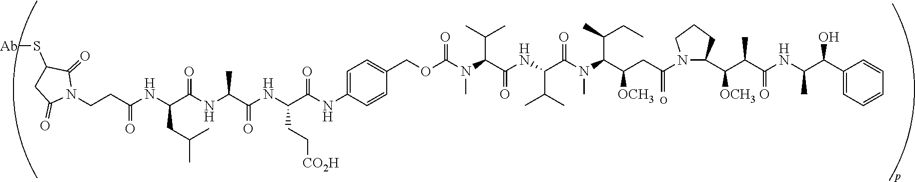
or a pharmaceutically acceptable salt thereof,
wherein Ab is the antibody h12F3 and p denotes a number from 1 to 12.

32. An isolated nucleic acid encoding the antigen binding protein or fragment of claim 1.

33. A vector comprising the nucleic acid of claim 32.

34. A host cell comprising the vector of claim 33.

35. The host cell of claim 34, wherein the host cell is a CHO cell.

36. A host cell that produces the antigen binding protein or fragment of claim 1.

37. A method for making an antigen binding protein or fragment thereof, the method comprising culturing the host cell of claim 36 under conditions suitable for production of the antigen binding protein.

38. The method of claim 37, further comprising recovering the antigen binding protein or fragment produced by the host cell.

39. The method of claim 37 wherein the host cell is a CHO cell.

40. An antigen binding protein or fragment thereof produced by the method of claim 37.

41. A pharmaceutical composition comprising the antigen binding protein or fragment of claim 1 and a pharmaceutically acceptable carrier.

42. A method of treating an ALPP and/or ALPPL2-expressing cancer in an individual comprising administering to an individual in need thereof an effective amount of an antigen binding protein or fragment of claim 1.

43. The method of claim 42, wherein the cancer is ovarian cancer, lung cancer, endometrial cancer, bladder cancer, gastric cancer, or testicular cancer.

44. The method of claim 43, wherein the cancer is ovarian cancer.

45-54. (canceled)

55. An ALPP and/or ALPPL2-binding antigen binding protein or fragment thereof that is capable of binding to one or more amino acids of a peptide comprising SEQ ID NO: 73 and/or SEQ ID NO: 74.
